SEQUENCE LISTING (92 sequences)

SEQ ID NO: 1 - DNA, 23 bp, synthetic construct
  source 1..23: mol_type = other DNA
ttggtggcagcaccggcacctgg

SEQ ID NO: 2 - DNA, 50 bp, synthetic construct
  source 1..50: mol_type = other DNA
tcgaagtagtgattgttggtggcagcaccggcaccgttttagagctagaa

SEQ ID NO: 3 - DNA, 50 bp, synthetic construct
  source 1..50: mol_type = other DNA
ttctagctctaaaacggtgccggtgctgccaccaacaatcactacttcga

SEQ ID NO: 4 - DNA, 25 bp, synthetic construct
  source 1..25: mol_type = other DNA
agggataacatgagattttgactgg

SEQ ID NO: 5 - DNA, 20 bp, synthetic construct
  source 1..20: mol_type = other DNA
cagagatcgggagaatgggc

SEQ ID NO: 6 - DNA, 23 bp, synthetic construct
  source 1..23: mol_type = other DNA
gcagcaacactggcaccacctgg

SEQ ID NO: 7 - DNA, 50 bp, synthetic construct
  source 1..50: mol_type = other DNA
tcgaagtagtgattggcagcaacactggcaccaccgttttagagctagaa

SEQ ID NO: 8 - DNA, 50 bp, synthetic construct
  source 1..50: mol_type = other DNA
ttctagctctaaaacggtggtgccagtgttgctgccaatcactacttcga

SEQ ID NO: 9 - DNA, 21 bp, synthetic construct
  source 1..21: mol_type = other DNA
acacgtgtctccaagttgtgt

SEQ ID NO: 10 - DNA, 20 bp, synthetic construct
  source 1..20: mol_type = other DNA
acgcgtttgtgattgtgctc

SEQ ID NO: 11 - DNA, 20 bp, synthetic construct
  source 1..20: mol_type = other DNA
ttggggctcacaccaaactt

SEQ ID NO: 12 - DNA, 20 bp, synthetic construct
  source 1..20: mol_type = other DNA
cgatcgccttcttttgctcg

SEQ ID NO: 13 - DNA, 3464 bp, synthetic construct
  source 1..3464: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgtttaaacggcgcgccccaatgtccctaggttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgctagcggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcagaagacccgttttagagctagaaatagcaagttaaaataaggctagtccgttatcaacttgaaaaagtggcaccgagtcggtgctttttttactagtttaattaaggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 14508 bp, synthetic construct
  source 1..14508: mol_type = other DNA
agtactttaaagtactttaaagtactttaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcggtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggtagaggcatcttgaacgatagcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcaggcatgcaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaatggcccctaagaagaagagaaaggtcggtattcacggcgttcctgcggcgatggacaagaagtatagtattggtctggacattgggacgaattccgttggctgggccgtgatcaccgatgagtacaaggtcccttccaagaagtttaaggttctggggaacaccgatcggcacagcatcaagaagaatctcattggagccctcctgttcgactcaggcgagaccgccgaagcaacaaggctcaagagaaccgcaaggagacggtatacaagaaggaagaataggatctgctacctgcaggagattttcagcaacgaaatggcgaaggtggacgattcgttctttcatagattggaggagagtttcctcgtcgaggaagataagaagcacgagaggcatcctatctttggcaacattgtcgacgaggttgcctatcacgaaaagtaccccacaatctatcatctgcggaagaagcttgtggactcgactgataaggcggaccttagattgatctacctcgctctggcacacatgattaagttcaggggccattttctgatcgagggggatcttaacccggacaatagcgatgtggacaagttgttcatccagctcgtccaaacctacaatcagctctttgaggaaaacccaattaatgcttcaggcgtcgacgccaaggcgatcctgtctgcacgcctttcaaagtctcgccggcttgagaacttgatcgctcaactcccgggcgaaaagaagaacggcttgttcgggaatctcattgcactttcgttggggctcacaccaaacttcaagagtaattttgatctcgctgaggacgcaaagctgcagctttccaaggacacttatgacgatgacctggataaccttttggcccaaatcggcgatcagtacgcggacttgttcctcgccgcgaagaatttgtcggacgcgatcctcctgagtgatattctccgcgtgaacaccgagattacaaaggccccgctctcggcgagtatgatcaagcgctatgacgagcaccatcaggatctgacccttttgaaggctttggtccggcagcaactcccagagaagtacaaggaaatcttctttgatcaatccaagaacggctacgctggttatattgacggcggggcatcgcaggaggaattctacaagtttatcaagccaattctggagaagatggatggcacagaggaactcctggtgaagctcaatagggaggaccttttgcggaagcaaagaactttcgataacggcagcatccctcaccagattcatctcggggagctgcacgccatcctgagaaggcaggaagacttctacccctttcttaaggataaccgggagaagatcgaaaagattctgacgttcagaattccgtactatgtcggaccactcgcccggggtaattccagatttgcgtggatgaccagaaagagcgaggaaaccatcacaccttggaacttcgaggaagtggtcgataagggcgcttccgcacagagcttcattgagcgcatgacaaattttgacaagaacctgcctaatgagaaggtccttcccaagcattccctcctgtacgagtatttcactgtttataacgaactcacgaaggtgaagtatgtgaccgagggaatgcgcaagcccgccttcctgagcggcgagcaaaagaaggcgatcgtggaccttttgtttaagaccaatcggaaggtcacagttaagcagctcaaggaggactacttcaagaagattgaatgcttcgattccgttgagatcagcggcgtggaagacaggtttaacgcgtcactggggacttaccacgatctcctgaagatcattaaggataaggacttcttggacaacgaggaaaatgaggatatcctcgaagacattgtcctgactcttacgttgtttgaggatagggaaatgatcgaggaacgcttgaagacgtatgcccatctcttcgatgacaaggttatgaagcagctcaagagaagaagatacaccggatggggaaggctgtcccgcaagcttatcaatggcattagagacaagcaatcagggaagacaatccttgactttttgaagtctgatggcttcgcgaacaggaattttatgcagctgattcacgatgactcacttactttcaaggaggatatccagaaggctcaagtgtcgggacaaggtgacagtctgcacgagcatatcgccaaccttgcgggatctcctgcaatcaagaagggtattctgcagacagtcaaggttgtggatgagcttgtgaaggtcatgggacggcataagcccgagaacatcgttattgagatggccagagaaaatcagaccacacaaaagggtcagaagaactcgagggagcgcatgaagcgcatcgaggaaggcattaaggagctggggagtcagatccttaaggagcacccggtggaaaacacgcagttgcaaaatgagaagctctatctgtactatctgcaaaatggcagggatatgtatgtggaccaggagttggatattaaccgcctctcggattacgacgtcgatcatatcgttcctcagtccttccttaaggatgacagcattgacaataaggttctcaccaggtccgacaagaaccgcgggaagtccgataatgtgcccagcgaggaagtcgttaagaagatgaagaactactggaggcaacttttgaatgccaagttgatcacacagaggaagtttgataacctcactaaggccgagcgcggaggtctcagcgaactggacaaggcgggcttcattaagcggcaactggttgagactagacagatcacgaagcacgtggcgcagattctcgattcacgcatgaacacgaagtacgatgagaatgacaagctgatccgggaagtgaaggtcatcaccttgaagtcaaagctcgtttctgacttcaggaaggatttccaattttataaggtgcgcgagatcaacaattatcaccatgctcatgacgcatacctcaacgctgtggtcggaacagcattgattaagaagtacccgaagctcgagtccgaattcgtgtacggtgactataaggtttacgatgtgcgcaagatgatcgccaagtcagagcaggaaattggcaaggccactgcgaagtatttcttttactctaacattatgaatttctttaagactgagatcacgctggctaatggcgaaatccggaagagaccacttattgagaccaacggcgagacaggggaaatcgtgtgggacaaggggagggatttcgccacagtccgcaaggttctctctatgcctcaagtgaatattgtcaagaagactgaagtccagacgggcgggttctcaaaggaatctattctgcccaagcggaactcggataagcttatcgccagaaagaaggactgggacccgaagaagtatggaggtttcgactcaccaacggtggcttactctgtcctggttgtggcaaaggtggagaagggaaagtcaaagaagctcaagtctgtcaaggagctcctgggtatcaccattatggagaggtccagcttcgaaaagaatccgatcgattttctcgaggcgaagggatataaggaagtgaagaaggacctgatcattaagcttccaaagtacagtcttttcgagttggaaaacggcaggaagcgcatgttggcttccgcaggagagctccagaagggtaacgagcttgctttgccgtccaagtatgtgaacttcctctatctggcatcccactacgagaagctcaagggcagcccagaggataacgaacagaagcaactgtttgtggagcaacacaagcattatcttgacgagatcattgaacagatttcggagttcagtaagcgcgtcatcctcgccgacgcgaatttggataaggttctctcagcctacaacaagcaccgggacaagcctatcagagagcaggcggaaaatatcattcatctcttcaccctgacaaaccttggggctcccgctgcattcaagtattttgacactacgattgatcggaagagatacacttctacgaaggaggtgctggatgcaacccttatccaccaatcgattactggcctctacgagacgcggatcgacttgagtcagctcgggggggataagagaccagcggcaaccaagaaggcaggacaagcgaagaagaagaagtaggttaacgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcttaattaaggatccgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggagcttgagcttggatcagattgtcgtttcccgccttcaggcgcgccgtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaa

SEQ ID NO: 15 - DNA, 2678 bp, Glycine max
  source 1..2678: mol_type = genomic DNA
acagccaataagattacatgaaaggaaagcatatatattaagggataacatgagattttgactggtgcatgtatatagaatctacatggacccaaaattacacgtgtctccaagttgtgcctccatagcaatacgaacaaagcctcaacatctctttaatgattccaaaaggcaaaggaagcaacatgcacaggtccaacaaaaaaaaaaaaaaaaaacctctctcctattcaaccccaaaacacaaccaaccacaagataagcaagaacttgtgcacgccactaacttgctgccacgatggcacccttgattctcccccaacactacttggttctcctcactgaactcaacttctcactcacactcacttctcttcctccaaattagttccaactccaagttagttccaacacaaacacaaacacacacacatacataaaccaagcaaagaaagactctacaaacacttcactactcatacaatattttcagacacaacatgttgaaggaaggcaccaacaacgttggtggcagcaccggcacctggtcacatgtctgcgacacgtgccggtcagcgccatgcgtactgtactgccatgcagactcagcatacctttgctcatcctgcgatgctcgtgtccatgcggctaaccgtgtggcctcaagacacgagcgtgtgtgggtgtgcgaagcgtgtgaacgtgctcccgcagcgtttctatgcaaagccgacgcagcttctctttgttcttcctgtgatgctgacattcactcagcaaaccctctcgctagccgccaccaccgcgtgcccattctcccgatctctggttccctcttcggggaaccagagcatgaacgcgtgtacgcgttcgtgaatgaagtggaagcggaggaggaagaggaagaggtttttgatgagtatgatgaggttgaagcagcttcgtggttgttgccacatcctatgaaaaatgataaaattgacgagaatggtggtgataagggttttttgtttggtgatgagtatttcgacaaccttgttgattgtaactcatgtggtcacaataacaaccagtttagcaacgtttatgatcagcaccagcagaattacagcaacactgtccctcagaactatgcagtggttccagttcaggtgccgcagcattttcaaccgggtttggactttgactcatcaaaagctgggttcagttacgatggttctcttagtcaaagtgtaagttaccttctttttacttctatgtgtttctttttggtttgttgtttatatcaatggggttttcccttctttttttttttttttttctttttgcaaactctttggatcattcatccattggattatactactttattagattttgccttgaaacctgtgctaactcttccttctaattgacagtttctttttctccattcgtatgaaaccaaggttttaaactgcagtcatagtcatagtcaagattatagtttattcaaaaacattagcattgtaatacaattgtactgtaattgtcgtcgtgaaaaagttaaaaaaaagtttaaaaaatctcaatcgcataccttctataaaaatcttgattgaaaccaaagtgggtttcataagtagtttcttttacaagaaaaactagcaataaagtcaactactctagatccattgccatacatgtgaccttgatccattccacaacgtttcgtatttctttgcctacccttttcccattgactacttttctgtcttgagagacatttattttatttttttcgtatgcaatagctttccctttttgatgtcttgtatttgctagttatagttgtgattcctcataaacagatacttacttttatagggtaacttagcttttcaccaattttcgcccatacatagttagacttccctaaactttgtttcctcatttcttcaactataatttgatcaattgtatattaatttcccataattttcagcttcaattaaaaaatttgagcctaagtgcctgttttattaaagccaaacaatggggggtttaatattttaaaaactttcttctatatttcttttacatttcctctgttttctaatatatcttttcttttttctattacatcatctttgatatattttcacctttttcttcttactttggggacaaacattattgtttcacaaaattggttagctactaatagtttattgccttacctgtaatctctcaaacttcatacacgtttttgtgttaatcttttatatttcatttttaatttaggtttcggtttcatcgatggatgttggtgttgtactcgaatcaacaataagtgacatctcaatgtcccactcaaagtcgccaatagggacaactgacctatttcctccccttcccatgccttcacatctcacaccaatggacagagaggcaagagtcctaagatacagggagaaaaagaagacaagaaaatttgagaagaaaataaggtatgcctcaaggaaggcctatgcagagactagaccccgcataaagggtcgttttgcaaagagaaccgatgtagaagctgaagtggaccagatgttctccacaacactattcactgaagttggaggtagcatttttcccactttctagaatggaaagaaagtaatggccaggccataaaagagaaggtt

SEQ ID NO: 16 - DNA, 1086 bp, Glycine max
  source 1..1086: mol_type = other DNA
atgttgaaggaaggcaccaacaacgttggtggcagcaccggcacctggtcacatgtctgcgacacgtgccggtcagcgccatgcgtactgtactgccatgcagactcagcatacctttgctcatcctgcgatgctcgtgtccatgcggctaaccgtgtggcctcaagacacgagcgtgtgtgggtgtgcgaagcgtgtgaacgtgctcccgcagcgtttctatgcaaagccgacgcagcttctctttgttcttcctgtgatgctgacattcactcagcaaaccctctcgctagccgccaccaccgcgtgcccattctcccgatctctggttccctcttcggggaaccagagcatgaacgcgtgtacgcgttcgtgaatgaagtggaagcggaggaggaagaggaagaggtttttgatgagtatgatgaggttgaagcagcttcgtggttgttgccacatcctatgaaaaatgataaaattgacgagaatggtggtgataagggttttttgtttggtgatgagtatttcgacaaccttgttgattgtaactcatgtggtcacaataacaaccagtttagcaacgtttatgatcagcaccagcagaattacagcaacactgtccctcagaactatgcagtggttccagttcaggtgccgcagcattttcaaccgggtttggactttgactcatcaaaagctgggttcagttacgatggttctcttagtcaaagtgtttcggtttcatcgatggatgttggtgttgtactcgaatcaacaataagtgacatctcaatgtcccactcaaagtcgccaatagggacaactgacctatttcctccccttcccatgccttcacatctcacaccaatggacagagaggcaagagtcctaagatacagggagaaaaagaagacaagaaaatttgagaagaaaataaggtatgcctcaaggaaggcctatgcagagactagaccccgcataaagggtcgttttgcaaagagaaccgatgtagaagctgaagtggaccagatgttctccacaacactattcactgaagttggaggtagcatttttcccactttctag

SEQ ID NO: 17 - protein, 361 aa, Glycine max
MLKEGTNNVGGSTGTWSHVCDTCRSAPCVLYCHADSAYLCSSCDARVHAANRVASRHERVWVCEACERAPAAFLCKADAASLCSSCDADIHSANPLASRHHRVPILPISGSLFGEPEHERVYAFVNEVEAEEEEEEVFDEYDEVEAASWLLPHPMKNDKIDENGGDKGFLFGDEYFDNLVDCNSCGHNNNQFSNVYDQHQQNYSNTVPQNYAVVPVQVPQHFQPGLDFDSSKAGFSYDGSLSQSVSVSSMDVGVVLESTISDISMSHSKSPIGTTDLFPPLPMPSHLTPMDREARVLRYREKKKTRKFEKKIRYASRKAYAETRPRIKGRFAKRTDVEAEVDQMFSTTLFTEVGGSIFPTF

SEQ ID NO: 18 - DNA, 2280 bp, Glycine max
  source 1..2280: mol_type = other DNA
acagccaataagattacatgaaaggaaagcatatatattaagggataacatgagattttgactggtgcatgtatatagaatctacatggacccaaaattacacgtgtctccaagttgtgcctccatagcaatacgaacaaagcctcaacatctctttaatgattccaaaaggcaaaggaagcatgcgtactgtactgccatgcagactcagcatacctttgctcatcctgcgatgctcgtgtccatgcggctaaccgtgtggcctcaagacacgagcgtgtgtgggtgtgcgaagcgtgtgaacgtgctcccgcagcgtttctatgcaaagccgacgcagcttctctttgttcttcctgtgatgctgacattcactcagcaaaccctctcgctagccgccaccaccgcgtgcccattctcccgatctctggttccctcttcggggaaccagagcatgaacgcgtgtacgcgttcgtgaatgaagtggaagcggaggaggaagaggaagaggtttttgatgagtatgatgaggttgaagcagcttcgtggttgttgccacatcctatgaaaaatgataaaattgacgagaatggtggtgataagggttttttgtttggtgatgagtatttcgacaaccttgttgattgtaactcatgtggtcacaataacaaccagtttagcaacgtttatgatcagcaccagcagaattacagcaacactgtccctcagaactatgcagtggttccagttcaggtgccgcagcattttcaaccgggtttggactttgactcatcaaaagctgggttcagttacgatggttctcttagtcaaagtgtaagttaccttctttttacttctatgtgtttctttttggtttgttgtttatatcaatggggttttcccttctttttttttttttttttctttttgcaaactctttggatcattcatccattggattatactactttattagattttgccttgaaacctgtgctaactcttccttctaattgacagtttctttttctccattcgtatgaaaccaaggttttaaactgcagtcatagtcatagtcaagattatagtttattcaaaaacattagcattgtaatacaattgtactgtaattgtcgtcgtgaaaaagttaaaaaaaagtttaaaaaatctcaatcgcataccttctataaaaatcttgattgaaaccaaagtgggtttcataagtagtttcttttacaagaaaaactagcaataaagtcaactactctagatccattgccatacatgtgaccttgatccattccacaacgtttcgtatttctttgcctacccttttcccattgactacttttctgtcttgagagacatttattttatttttttcgtatgcaatagctttccctttttgatgtcttgtatttgctagttatagttgtgattcctcataaacagatacttacttttatagggtaacttagcttttcaccaattttcgcccatacatagttagacttccctaaactttgtttcctcatttcttcaactataatttgatcaattgtatattaatttcccataattttcagcttcaattaaaaaatttgagcctaagtgcctgttttattaaagccaaacaatggggggtttaatattttaaaaactttcttctatatttcttttacatttcctctgttttctaatatatcttttcttttttctattacatcatctttgatatattttcacctttttcttcttactttggggacaaacattattgtttcacaaaattggttagctactaatagtttattgccttacctgtaatctctcaaacttcatacacgtttttgtgttaatcttttatatttcatttttaatttaggtttcggtttcatcgatggatgttggtgttgtactcgaatcaacaataagtgacatctcaatgtcccactcaaagtcgccaatagggacaactgacctatttcctccccttcccatgccttcacatctcacaccaatggacagagaggcaagagtcctaagatacagggagaaaaagaagacaagaaaatttgagaagaaaataaggtatgcctcaaggaaggcctatgcagagactagaccccgcataaagggtcgttttgcaaagagaaccgatgtagaagctgaagtggaccagatgttctccacaacactattcactgaagttggaggtagcatttttcccactttctagaatggaaagaaagtaatggccaggccataaaagagaaggtt

SEQ ID NO: 19 - DNA, 1007 bp, Glycine max
  source 1..1007: mol_type = other DNA
catgcgtactgtactgccatgcagactcagcatacctttgctcatcctgcgatgctcgtgtccatgcggctaaccgtgtggcctcaagacacgagcgtgtgtgggtgtgcgaagcgtgtgaacgtgctcccgcagcgtttctatgcaaagccgacgcagcttctctttgttcttcctgtgatgctgacattcactcagcaaaccctctcgctagccgccaccaccgcgtgcccattctcccgatctctggttccctcttcggggaaccagagcatgaacgcgtgtacgcgttcgtgaatgaagtggaagcggaggaggaagaggaagaggtttttgatgagtatgatgaggttgaagcagcttcgtggttgttgccacatcctatgaaaaatgataaaattgacgagaatggtggtgataagggttttttgtttggtgatgagtatttcgacaaccttgttgattgtaactcatgtggtcacaataacaaccagtttagcaacgtttatgatcagcaccagcagaattacagcaacactgtccctcagaactatgcagtggttccagttcaggtgccgcagcattttcaaccgggtttggactttgactcatcaaaagctgggttcagttacgatggttctcttagtcaaagtgtttcggtttcatcgatggatgttggtgttgtactcgaatcaacaataagtgacatctcaatgtcccactcaaagtcgccaatagggacaactgacctatttcctccccttcccatgccttcacatctcacaccaatggacagagaggcaagagtcctaagatacagggagaaaaagaagacaagaaaatttgagaagaaaataaggtatgcctcaaggaaggcctatgcagagactagaccccgcataaagggtcgttttgcaaagagaaccgatgtagaagctgaagtggaccagatgttctccacaacactattcactgaagttggaggtagcatttttcccactttctag

SEQ ID NO: 20 - protein, 23 aa, Glycine max
MRTVLPCRLSIPLLILRCSCPCG

SEQ ID NO: 21 - DNA, 2710 bp, Glycine max
  source 1..2710: mol_type = genomic DNA
accaaacacttgctttaatccttttagagccaataagattactgagaggaaaggatatattaaggggtaacaagagattctaactggtgcaagtatagaatctacatggacccaaatgacacgtgtctccaagttgtgtcaccatagcaatacgaacaaagcctcagcatctctttaatgattccaaaaggcaaagaagcaacatgcatggacaggtccaacaccaaaacaaaaaaccctctctcctaatcaaccctgaaacacaaccaaccacaagataagcaagaacttgtgcacgccactaacttgctgccacgttggcactcttgattctccccccaacactacttggttcacctcactaaactcaatttctcactcacactcacttcagttcaattcacttcccttcctccaaattagttccaacacaaaaaccaagcaaagaaaaagactctaccgaaacacttcactactcatacaatattttcagacacaacatgttgaaggaaggcaccaacaacgttggtggcagcaacactggcaccacctggtcacgtgtctgtgacacgtgcctgtctgcgccatgcgtgctgtactgccatgcagactcagcatatctttgctcttcctgtgatgctcgtgtgcatgcagctaaccgtgtggcctcgagacacaagcgtgtgtgggtgtgcgaagcgtgtgagcgtgctccggcggcgtttctatgcaaagctgacgcagcttctctttgttcttcctgtgatgctgacattcactcagcaaaccctctcgctagccgccacaaccgtgttcccattctcccgatctcaggttccctcttcagggaaccagagcacaatcacaaacgcgtggaacacgcgttcgtgaatgaggttgaggaggaagaggaaggggtttttgatgagtatgaagacgaggttgaagctgcttcgtggttgttgccacatcctatgaaaaataatgatgaaattgaggagaatgattgtggtgacgagggttttttgtttgttgatgagtatttggacaatcttgttgattgttgtaactcatgtggtcacaatgacaaccagtttagcaacgtttatcagcaccagcagaattacaacactgtccctcagaactatgtagtggttccagttcaggtgccgcagcattttcaacccggtttggactttgactcatcaaaagctgggttcagttacgatggttctcttagtcaaagtgtaagactcttcttttcaattacttctgtgtttctttttcggtttgttttttatatgaatagtgttttccctttttctctttctttttcttttcccagactctttggatcattcatccattggattatactttatcagattttgattttgccttgacacctttgctaactcttccttccaattggcagtttctttcctttctccgttcatatgaaactagggttttaaattatactcatgatcaataatttggttgcaatttttgatattgtagtacaattgctattgttgttgtgaaaagttaaaaaaatcttaatgttgtaaccgaaatctcaatcacatactgtttgtaaaatccttggttgaaatcaaagtgggtttcccaagtagcttcttttactagaaacgctagcaaagtcaactactctagttcaattgccatatatgtgactaatttgattcattccacaactcttcatatttctttgactacccttttaccgttcactttgcggtcttgagagacttttatttattttcttttttgtcgtatataagatcagctttgcctttatggtgtcttttatttgctagttatagttgtgattcctcttaaactttgtttcctcatttcatcaactgtatattaatttcccataatttttcagcttcaattaaaaaaattgagcctaagtgcctgatttattaaagccacaaaatgggtggtttaattttttcgacacctttcttttatatttcttttactcttcttctcttttctttttctattacatcatctataatatatcttcacctttttcttcttactttggggacaaacatttttgtttcacaaagctggttagctactagtttattgcttagaaaatttatagccttacctgtgatctctcaaacttcatacacgattttgtgttaatcttttatatttcatttttaatttaggtttcggtttcatcaatggatgttggagttgtacccgaatcaacagtaagtggcatctcaatgtcccactcaaagtcaccaatagggacaaatgacctatttcctccccttctcatgccttcacatctcacaccaatggacagagaggcaagagtcctaagatatagggagaaaaagaagacaagaaagttcgagaagaaaataagatatgcctcaaggaaggcctatgcagagactagaccccgcataaagggtcgttttgcaaagagaactgatgtagaagctgaagtggatcagatgttctccacaaaactattcaatgaagttggaggtagcatttttcccactttctagaatggaaagaaagtaattgccaagccatagaagagaaggttgggaaccacacatcattggaagaacttcatgtaatttttatgtagtctaattagtttggtattatatttacttctatcttgtgctttataaatataaagtat

SEQ ID NO: 22 - DNA, 1101 bp, Glycine max
  source 1..1101: mol_type = other DNA
atgttgaaggaaggcaccaacaacgttggtggcagcaacactggcaccacctggtcacgtgtctgtgacacgtgcctgtctgcgccatgcgtgctgtactgccatgcagactcagcatatctttgctcttcctgtgatgctcgtgtgcatgcagctaaccgtgtggcctcgagacacaagcgtgtgtgggtgtgcgaagcgtgtgagcgtgctccggcggcgtttctatgcaaagctgacgcagcttctctttgttcttcctgtgatgctgacattcactcagcaaaccctctcgctagccgccacaaccgtgttcccattctcccgatctcaggttccctcttcagggaaccagagcacaatcacaaacgcgtggaacacgcgttcgtgaatgaggttgaggaggaagaggaaggggtttttgatgagtatgaagacgaggttgaagctgcttcgtggttgttgccacatcctatgaaaaataatgatgaaattgaggagaatgattgtggtgacgagggttttttgtttgttgatgagtatttggacaatcttgttgattgttgtaactcatgtggtcacaatgacaaccagtttagcaacgtttatcagcaccagcagaattacaacactgtccctcagaactatgtagtggttccagttcaggtgccgcagcattttcaacccggtttggactttgactcatcaaaagctgggttcagttacgatggttctcttagtcaaagtgtttcggtttcatcaatggatgttggagttgtacccgaatcaacagtaagtggcatctcaatgtcccactcaaagtcaccaatagggacaaatgacctatttcctccccttctcatgccttcacatctcacaccaatggacagagaggcaagagtcctaagatatagggagaaaaagaagacaagaaagttcgagaagaaaataagatatgcctcaaggaaggcctatgcagagactagaccccgcataaagggtcgttttgcaaagagaactgatgtagaagctgaagtggatcagatgttctccacaaaactattcaatgaagttggaggtagcatttttcccactttctag

SEQ ID NO: 23 - protein, 366 aa, Glycine max
MLKEGTNNVGGSNTGTTWSRVCDTCLSAPCVLYCHADSAYLCSSCDARVHAANRVASRHKRVWVCEACERAPAAFLCKADAASLCSSCDADIHSANPLASRHNRVPILPISGSLFREPEHNHKRVEHAFVNEVEEEEEGVFDEYEDEVEAASWLLPHPMKNNDEIEENDCGDEGFLFVDEYLDNLVDCCNSCGHNDNQFSNVYQHQQNYNTVPQNYVVVPVQVPQHFQPGLDFDSSKAGFSYDGSLSQSVSVSSMDVGVVPESTVSGISMSHSKSPIGTNDLFPPLLMPSHLTPMDREARVLRYREKKKTRKFEKKIRYASRKAYAETRPRIKGRFAKRTDVEAEVDQMFSTKLFNEVGGSIFPTF

SEQ ID NO: 24 - DNA, 2709 bp, Glycine max
  source 1..2709: mol_type = other DNA
accaaacacttgctttaatccttttagagccaataagattactgagaggaaaggatatattaaggggtaacaagagattctaactggtgcaagtatagaatctacatggacccaaatgacacgtgtctccaagttgtgtcaccatagcaatacgaacaaagcctcagcatctctttaatgattccaaaaggcaaagaagcaacatgcatggacaggtccaacaccaaaacaaaaaaccctctctcctaatcaaccctgaaacacaaccaaccacaagataagcaagaacttgtgcacgccactaacttgctgccacgttggcactcttgattctccccccaacactacttggttcacctcactaaactcaatttctcactcacactcacttcagttcaattcacttcccttcctccaaattagttccaacacaaaaaccaagcaaagaaaaagactctaccgaaacacttcactactcatacaatattttcagacacaacatgttgaaggaaggcaccaacaacgttggtggcagcaacactggcacacctggtcacgtgtctgtgacacgtgcctgtctgcgccatgcgtgctgtactgccatgcagactcagcatatctttgctcttcctgtgatgctcgtgtgcatgcagctaaccgtgtggcctcgagacacaagcgtgtgtgggtgtgcgaagcgtgtgagcgtgctccggcggcgtttctatgcaaagctgacgcagcttctctttgttcttcctgtgatgctgacattcactcagcaaaccctctcgctagccgccacaaccgtgttcccattctcccgatctcaggttccctcttcagggaaccagagcacaatcacaaacgcgtggaacacgcgttcgtgaatgaggttgaggaggaagaggaaggggtttttgatgagtatgaagacgaggttgaagctgcttcgtggttgttgccacatcctatgaaaaataatgatgaaattgaggagaatgattgtggtgacgagggttttttgtttgttgatgagtatttggacaatcttgttgattgttgtaactcatgtggtcacaatgacaaccagtttagcaacgtttatcagcaccagcagaattacaacactgtccctcagaactatgtagtggttccagttcaggtgccgcagcattttcaacccggtttggactttgactcatcaaaagctgggttcagttacgatggttctcttagtcaaagtgtaagactcttcttttcaattacttctgtgtttctttttcggtttgttttttatatgaatagtgttttccctttttctctttctttttcttttcccagactctttggatcattcatccattggattatactttatcagattttgattttgccttgacacctttgctaactcttccttccaattggcagtttctttcctttctccgttcatatgaaactagggttttaaattatactcatgatcaataatttggttgcaatttttgatattgtagtacaattgctattgttgttgtgaaaagttaaaaaaatcttaatgttgtaaccgaaatctcaatcacatactgtttgtaaaatccttggttgaaatcaaagtgggtttcccaagtagcttcttttactagaaacgctagcaaagtcaactactctagttcaattgccatatatgtgactaatttgattcattccacaactcttcatatttctttgactacccttttaccgttcactttgcggtcttgagagacttttatttattttcttttttgtcgtatataagatcagctttgcctttatggtgtcttttatttgctagttatagttgtgattcctcttaaactttgtttcctcatttcatcaactgtatattaatttcccataatttttcagcttcaattaaaaaaattgagcctaagtgcctgatttattaaagccacaaaatgggtggtttaattttttcgacacctttcttttatatttcttttactcttcttctcttttctttttctattacatcatctataatatatcttcacctttttcttcttactttggggacaaacatttttgtttcacaaagctggttagctactagtttattgcttagaaaatttatagccttacctgtgatctctcaaacttcatacacgattttgtgttaatcttttatatttcatttttaatttaggtttcggtttcatcaatggatgttggagttgtacccgaatcaacagtaagtggcatctcaatgtcccactcaaagtcaccaatagggacaaatgacctatttcctccccttctcatgccttcacatctcacaccaatggacagagaggcaagagtcctaagatatagggagaaaaagaagacaagaaagttcgagaagaaaataagatatgcctcaaggaaggcctatgcagagactagaccccgcataaagggtcgttttgcaaagagaactgatgtagaagctgaagtggatcagatgttctccacaaaactattcaatgaagttggaggtagcatttttcccactttctagaatggaaagaaagtaattgccaagccatagaagagaaggttgggaaccacacatcattggaagaacttcatgtaatttttatgtagtctaattagtttggtattatatttacttctatcttgtgctttataaatataaagtat

SEQ ID NO: 25 - DNA, 1100 bp, Glycine max
  source 1..1100: mol_type = other DNA
atgttgaaggaaggcaccaacaacgttggtggcagcaacactggcacacctggtcacgtgtctgtgacacgtgcctgtctgcgccatgcgtgctgtactgccatgcagactcagcatatctttgctcttcctgtgatgctcgtgtgcatgcagctaaccgtgtggcctcgagacacaagcgtgtgtgggtgtgcgaagcgtgtgagcgtgctccggcggcgtttctatgcaaagctgacgcagcttctctttgttcttcctgtgatgctgacattcactcagcaaaccctctcgctagccgccacaaccgtgttcccattctcccgatctcaggttccctcttcagggaaccagagcacaatcacaaacgcgtggaacacgcgttcgtgaatgaggttgaggaggaagaggaaggggtttttgatgagtatgaagacgaggttgaagctgcttcgtggttgttgccacatcctatgaaaaataatgatgaaattgaggagaatgattgtggtgacgagggttttttgtttgttgatgagtatttggacaatcttgttgattgttgtaactcatgtggtcacaatgacaaccagtttagcaacgtttatcagcaccagcagaattacaacactgtccctcagaactatgtagtggttccagttcaggtgccgcagcattttcaacccggtttggactttgactcatcaaaagctgggttcagttacgatggttctcttagtcaaagtgtttcggtttcatcaatggatgttggagttgtacccgaatcaacagtaagtggcatctcaatgtcccactcaaagtcaccaatagggacaaatgacctatttcctccccttctcatgccttcacatctcacaccaatggacagagaggcaagagtcctaagatatagggagaaaaagaagacaagaaagttcgagaagaaaataagatatgcctcaaggaaggcctatgcagagactagaccccgcataaagggtcgttttgcaaagagaactgatgtagaagctgaagtggatcagatgttctccacaaaactattcaatgaagttggaggtagcatttttcccactttctag

SEQ ID NO: 26 - protein, 129 aa, Glycine max
MLKEGTNNVGGSNTGTPGHVSVTRACLRHACCTAMQTQHIFALPVMLVCMQLTVWPRDTSVCGCAKRVSVLRRRFYAKLTQLLFVLPVMLTFTQQTLSLAATTVFPFSRSQVPSSGNQSTITNAWNTRS

SEQ ID NO: 27 - DNA, 1688 bp, Glycine max
  source 1..1688: mol_type = genomic DNA
aaagagcaagataggccaaaagctagcttatccatatatataatgcctatatcaatggacccccttgttcttggacgtataataggagatattttggatcccttcacgagttctgtttctctgagggttgtttataacaaccagtcatcggttatcaatagttgtgagttcaaaccttcccaaatcgtcaacaaaccaagaattaatattcgtggaaatgacctagggatcttttacactctggtatgcatgccaattgaagctagctccatatatattttttcttcatttgtctttccgaaattctaagtctctgtgatagtgttgttcaattttgacataatggtgttgcatgcattatgcagataatggtgaaccccgatgctcctagcccaagtgacccacatatgaaggaatatttgcattggtgagtaccttcgtctcttgcctctattatcatattgaattttgagaataagccggtatatatatatatatatatacttaattaaagcgtaacaacgtagtatataagcttacttatcttatacctaaaatttaatagttttgaatctctttttcttttcttgttactgtcattttttaacaattcgttaccatatgtagtatatatgtttaaataaataccttaattaagtatatattcataaaatcctcattatgcaattgaaaaaataccttaattaattcactagaaaactattaaaataaataaaaatagtttataaaatgcataacttaaatcatctttgtgtataaacgcaaattagttttataaaaactcttccatacagtcccttaatttacttacgaagtacaattgagtagctgatagaagaacgttagcacacataacttgtaaatatttctccccacacacagctctactcctttaattaattaatttactaaatttaaaataaataattagtaagagacactaaaaaaacacgatcaagttaatgtggttgtctcaatgtgacacccgaagaaaattgtagagattttgtttttagaagaaaattgtagagattatagattatagagttgtgtatttataacatttgttcttcacttgatgcctaggttggtaaccaatattccagcatctacaggggcaaccactggtatgcttacaacttttattttcaattctttttgttctaccttggtgcttttatatactttggttataacttttaaatgtatattataatattaatcgatactcatatcttatatcttgcattcttatcgattgatattacttactaattaatggaattttatatataggagaagagattgtggagtatgaaagtccacgaccaacttctgggattcatcggattgcttttgtgttgtttcgtcaatttgatagacaaattgtgcatgctccaagatggcgccaaaatttcaacactagagattttgctgaggtttacaaccttggatcaccagttgctgctgtgtatttcaactgtcaacgtgaaggtggttggggtggacgaaggagatgaagacccttcttcgatagaactaatttagcaaaaaaaccaacgttactacatatatcacgtttggtgcagtcacactgcgcacgttgtcctaataattaataaatactgtaatatttgaaacctcttcaataatttcaggcaagctgc

SEQ ID NO: 28 - protein, 175 aa, Glycine max
MPISMDPLVLGRIIGDILDPFTSSVSLRVVYNNQSSVINSCEFKPSQIVNKPRINIRGNDLGIFYTLIMVNPDAPSPSDPHMKEYLHWLVTNIPASTGATTGEEIVEYESPRPTSGIHRIAFVLFRQFDRQIVHAPRWRQNFNTRDFAEVYNLGSPVAAVYFNCQREGGWGGRRR

SEQ ID NO: 29 - DNA, 1688 bp, Glycine max; identical to SEQ ID NO: 27
  source 1..1688: mol_type = other DNA

SEQ ID NO: 30 - protein, 29 aa, Glycine max
MPISMDPLVLGNRRYFGSLHEFCFSEGCL

SEQ ID NO: 31 - DNA, 1689 bp, Glycine max
  source 1..1689: mol_type = other DNA
aaagagcaagataggccaaaagctagcttatccatatatataatgcctatatcaatggacccccttgttcttggacgtattaataggagatattttggatcccttcacgagttctgtttctctgagggttgtttataacaaccagtcatcggttatcaatagttgtgagttcaaaccttcccaaatcgtcaacaaaccaagaattaatattcgtggaaatgacctagggatcttttacactctggtatgcatgccaattgaagctagctccatatatattttttcttcatttgtctttccgaaattctaagtctctgtgatagtgttgttcaattttgacataatggtgttgcatgcattatgcagataatggtgaaccccgatgctcctagcccaagtgacccacatatgaaggaatatttgcattggtgagtaccttcgtctcttgcctctattatcatattgaattttgagaataagccggtatatatatatatatatatacttaattaaagcgtaacaacgtagtatataagcttacttatcttatacctaaaatttaatagttttgaatctctttttcttttcttgttactgtcattttttaacaattcgttaccatatgtagtatatatgtttaaataaataccttaattaagtatatattcataaaatcctcattatgcaattgaaaaaataccttaattaattcactagaaaactattaaaataaataaaaatagtttataaaatgcataacttaaatcatctttgtgtataaacgcaaattagttttataaaaactcttccatacagtcccttaatttacttacgaagtacaattgagtagctgatagaagaacgttagcacacataacttgtaaatatttctccccacacacagctctactcctttaattaattaatttactaaatttaaaataaataattagtaagagacactaaaaaaacacgatcaagttaatgtggttgtctcaatgtgacacccgaagaaaattgtagagattttgtttttagaagaaaattgtagagattatagattatagagttgtgtatttataacatttgttcttcacttgatgcctaggttggtaaccaatattccagcatctacaggggcaaccactggtatgcttacaacttttattttcaattctttttgttctaccttggtgcttttatatactttggttataacttttaaatgtatattataatattaatcgatactcatatcttatatcttgcattcttatcgattgatattacttactaattaatggaattttatatataggagaagagattgtggagtatgaaagtccacgaccaacttctgggattcatcggattgcttttgtgttgtttcgtcaatttgatagacaaattgtgcatgctccaagatggcgccaaaatttcaacactagagattttgctgaggtttacaaccttggatcaccagttgctgctgtgtatttcaactgtcaacgtgaaggtggttggggtggacgaaggagatgaagacccttcttcgatagaactaatttagcaaaaaaaccaacgttactacatatatcacgtttggtgcagtcacactgcgcacgttgtcctaataattaataaatactgtaatatttgaaacctcttcaataatttcaggcaagctgc

SEQ ID NO: 32 - protein, 31 aa, Glycine max
MPISMDPLVLGRINRRYFGSLHEFCFSEGCL

SEQ ID NO: 33 - DNA, 4206 bp, synthetic construct
  source 1..4206: mol_type = other DNA
atggcccctaagaagaagagaaaggtcggtattcacggcgttcctgcggcgatggacaagaagtatagtattggtctggacattgggacgaattccgttggctgggccgtgatcaccgatgagtacaaggtcccttccaagaagtttaaggttctggggaacaccgatcggcacagcatcaagaagaatctcattggagccctcctgttcgactcaggcgagaccgccgaagcaacaaggctcaagagaaccgcaaggagacggtatacaagaaggaagaataggatctgctacctgcaggagattttcagcaacgaaatggcgaaggtggacgattcgttctttcatagattggaggagagtttcctcgtcgaggaagataagaagcacgagaggcatcctatctttggcaacattgtcgacgaggttgcctatcacgaaaagtaccccacaatctatcatctgcggaagaagcttgtggactcgactgataaggcggaccttagattgatctacctcgctctggcacacatgattaagttcaggggccattttctgatcgagggggatcttaacccggacaatagcgatgtggacaagttgttcatccagctcgtccaaacctacaatcagctctttgaggaaaacccaattaatgcttcaggcgtcgacgccaaggcgatcctgtctgcacgcctttcaaagtctcgccggcttgagaacttgatcgctcaactcccgggcgaaaagaagaacggcttgttcgggaatctcattgcactttcgttggggctcacaccaaacttcaagagtaattttgatctcgctgaggacgcaaagctgcagctttccaaggacacttatgacgatgacctggataaccttttggcccaaatcggcgatcagtacgcggacttgttcctcgccgcgaagaatttgtcggacgcgatcctcctgagtgatattctccgcgtgaacaccgagattacaaaggccccgctctcggcgagtatgatcaagcgctatgacgagcaccatcaggatctgacccttttgaaggctttggtccggcagcaactcccagagaagtacaaggaaatcttctttgatcaatccaagaacggctacgctggttatattgacggcggggcatcgcaggaggaattctacaagtttatcaagccaattctggagaagatggatggcacagaggaactcctggtgaagctcaatagggaggaccttttgcggaagcaaagaactttcgataacggcagcatccctcaccagattcatctcggggagctgcacgccatcctgagaaggcaggaagacttctacccctttcttaaggataaccgggagaagatcgaaaagattctgacgttcagaattccgtactatgtcggaccactcgcccggggtaattccagatttgcgtggatgaccagaaagagcgaggaaaccatcacaccttggaacttcgaggaagtggtcgataagggcgcttccgcacagagcttcattgagcgcatgacaaattttgacaagaacctgcctaatgagaaggtccttcccaagcattccctcctgtacgagtatttcactgtttataacgaactcacgaaggtgaagtatgtgaccgagggaatgcgcaagcccgccttcctgagcggcgagcaaaagaaggcgatcgtggaccttttgtttaagaccaatcggaaggtcacagttaagcagctcaaggaggactacttcaagaagattgaatgcttcgattccgttgagatcagcggcgtggaagacaggtttaacgcgtcactggggacttaccacgatctcctgaagatcattaaggataaggacttcttggacaacgaggaaaatgaggatatcctcgaagacattgtcctgactcttacgttgtttgaggatagggaaatgatcgaggaacgcttgaagacgtatgcccatctcttcgatgacaaggttatgaagcagctcaagagaagaagatacaccggatggggaaggctgtcccgcaagcttatcaatggcattagagacaagcaatcagggaagacaatccttgactttttgaagtctgatggcttcgcgaacaggaattttatgcagctgattcacgatgactcacttactttcaaggaggatatccagaaggctcaagtgtcgggacaaggtgacagtctgcacgagcatatcgccaaccttgcgggatctcctgcaatcaagaagggtattctgcagacagtcaaggttgtggatgagcttgtgaaggtcatgggacggcataagcccgagaacatcgttattgagatggccagagaaaatcagaccacacaaaagggtcagaagaactcgagggagcgcatgaagcgcatcgaggaaggcattaaggagctggggagtcagatccttaaggagcacccggtggaaaacacgcagttgcaaaatgagaagctctatctgtactatctgcaaaatggcagggatatgtatgtggaccaggagttggatattaaccgcctctcggattacgacgtcgatcatatcgttcctcagtccttccttaaggatgacagcattgacaataaggttctcaccaggtccgacaagaaccgcgggaagtccgataatgtgcccagcgaggaagtcgttaagaagatgaagaactactggaggcaacttttgaatgccaagttgatcacacagaggaagtttgataacctcactaaggccgagcgcggaggtctcagcgaactggacaaggcgggcttcattaagcggcaactggttgagactagacagatcacgaagcacgtggcgcagattctcgattcacgcatgaacacgaagtacgatgagaatgacaagctgatccgggaagtgaaggtcatcaccttgaagtcaaagctcgtttctgacttcaggaaggatttccaattttataaggtgcgcgagatcaacaattatcaccatgctcatgacgcatacctcaacgctgtggtcggaacagcattgattaagaagtacccgaagctcgagtccgaattcgtgtacggtgactataaggtttacgatgtgcgcaagatgatcgccaagtcagagcaggaaattggcaaggccactgcgaagtatttcttttactctaacattatgaatttctttaagactgagatcacgctggctaatggcgaaatccggaagagaccacttattgagaccaacggcgagacaggggaaatcgtgtgggacaaggggagggatttcgccacagtccgcaaggttctctctatgcctcaagtgaatattgtcaagaagactgaagtccagacgggcgggttctcaaaggaatctattctgcccaagcggaactcggataagcttatcgccagaaagaaggactgggacccgaagaagtatggaggtttcgactcaccaacggtggcttactctgtcctggttgtggcaaaggtggagaagggaaagtcaaagaagctcaagtctgtcaaggagctcctgggtatcaccattatggagaggtccagcttcgaaaagaatccgatcgattttctcgaggcgaagggatataaggaagtgaagaaggacctgatcattaagcttccaaagtacagtcttttcgagttggaaaacggcaggaagcgcatgttggcttccgcaggagagctccagaagggtaacgagcttgctttgccgtccaagtatgtgaacttcctctatctggcatcccactacgagaagctcaagggcagcccagaggataacgaacagaagcaactgtttgtggagcaacacaagcattatcttgacgagatcattgaacagatttcggagttcagtaagcgcgtcatcctcgccgacgcgaatttggataaggttctctcagcctacaacaagcaccgggacaagcctatcagagagcaggcggaaaatatcattcatctcttcaccctgacaaaccttggggctcccgctgcattcaagtattttgacactacgattgatcggaagagatacacttctacgaaggaggtgctggatgcaacccttatccaccaatcgattactggcctctacgagacgcggatcgacttgagtcagctcgggggggataagagaccagcggcaaccaagaaggcaggacaagcgaagaagaagaagtag

SEQ ID NO: 34 - DNA, 446 bp, Arabidopsis thaliana
  source 1..446: mol_type = other DNA
ttcgttgaacaacggaaactcgacttgcctttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattg

SEQ ID NO: 35 - DNA, 519 bp, Glycine max
  source 1..519: mol_type = other DNA
atggcacgggagaaccctcttgttattgggggtgtgattggggatgttctcaatccttttacaatctccgtttcttttactatttcaatcaataatagggcgattagcaatggcttggaactgaggccctctcaagttgttaatcgccctagagtcactgttggtggtgaagacctaaggaccttctacacactggttatggtggatgcagatgcacctagccctagcaaccctgtcttgagggaataccttcactggatggtgacagatattccagctaccacaaatgcaagctttgggagagaggttgtgttttatgagagcccgaacccttcagcagggattcatcgacttgtgttcatattattccagcaactgggcagagacactgtcatcaccccagaatggcgccataatttcaattccagaaactttgctgaaattaataaccttgcacctgttgcagcagcttatgccaactgccaaagagagcgtggttgcggtggaaggagatattaa

SEQ ID NO: 36 - protein, 172 aa, Glycine max
MARENPLVIGGVIGDVLNPFTISVSFTISINNRAISNGLELRPSQVVNRPRVTVGGEDLRTFYTLVMVDADAPSPSNPVLREYLHWMVTDIPATTNASFGREVVFYESPNPSAGIHRLVFILFQQLGRDTVITPEWRHNFNSRNFAEINNLAPVAAAYANCQRERGCGGRRY

SEQ ID NO: 37 - DNA, 511 bp, Glycine max
  source 1..511: mol_type = other DNA
atggcacgggagaaccctcgggggtgtgattggggatgttctcaatccttttacaatctccgtttcttttactatttcaatcaataatagggcgattagcaatggcttggaactgaggccctctcaagttgttaatcgccctagagtcactgttggtggtgaagacctaaggaccttctacacactggttatggtggatgcagatgcacctagccctagcaaccctgtcttgagggaataccttcactggatggtgacagatattccagctaccacaaatgcaagctttgggagagaggttgtgttttatgagagcccgaacccttcagcagggattcatcgacttgtgttcatattattccagcaactgggcagagacactgtcatcaccccagaatggcgccataatttcaattccagaaactttgctgaaattaataaccttgcacctgttgcagcagcttatgccaactgccaaagagagcgtggttgcggtggaaggagatattaa

SEQ ID NO: 38 - protein, 28 aa, Glycine max
MARENPRGCDWGCSQSFYNLRFFYYFNQ

SEQ ID NO: 39 - DNA, 519 bp, Glycine max
  source 1..519: mol_type = other DNA
atggcacgggagaaccctcttgttattggtggtgtgattggggatgttctcaacccttttacaagctccgtttctttgactgtttcaatcaataatagggcgattagcaatggcttggaactcaggccctctcaagttgttaatcgccctagggttactgttggtggtgaagacctaaggaccttctacactctggttatggtggatgcagatgcacctagccctagcaaccctgtcttgagggaataccttcactggatggtgacagatattccagctaccacaaatgcaagctttgggagagaggttgtgttttatgagagcccgaacccttcagtagggattcatcgaatcgtgttcgtattgttccagcaattgggcagagacactgtcatcaccccagaatggcgccataatttcaattccagaaactttgctgaaattaataaccttgcacctgttgcagcagcttatgccaactgccaaagagagcgtggttgcggtggaaggagatattaa

SEQ ID NO: 40 - protein, 172 aa, Glycine max
MARENPLVIGGVIGDVLNPFTSSVSLTVSINNRAISNGLELRPSQVVNRPRVTVGGEDLRTFYTLVMVDADAPSPSNPVLREYLHWMVTDIPATTNASFGREVVFYESPNPSVGIHRIVFVLFQQLGRDTVITPEWRHNFNSRNFAEINNLAPVAAAYANCQRERGCGGRRY

SEQ ID NO: 41 - DNA, 520 bp, Glycine max
  source 1..520: mol_type = other DNA
atgggcacgggagaaccctcttgttattggtggtgtgattggggatgttctcaacccttttacaagctccgtttctttgactgtttcaatcaataatagggcgattagcaatggcttggaactcaggccctctcaagttgttaatcgccctagggttactgttggtggtgaagacctaaggaccttctacactctggttatggtggatgcagatgcacctagccctagcaaccctgtcttgagggaataccttcactggatggtgacagatattccagctaccacaaatgcaagctttgggagagaggttgtgttttatgagagcccgaacccttcagtagggattcatcgaatcgtgttcgtattgttccagcaattgggcagagacactgtcatcaccccagaatggcgccataatttcaattccagaaactttgctgaaattaataaccttgcacctgttgcagcagcttatgccaactgccaaagagagcgtggttgcggtggaaggagatattaa

SEQ ID NO: 42 - protein, 31 aa, Glycine max
MGTGEPSCYWWCDWGCSQPFYKLRFFDCFNQ

SEQ ID NO: 43 - DNA, 23 bp, synthetic construct
  source 1..23: mol_type = other DNA
cttgttcttggacgtataatagg

SEQ ID NO: 44 - DNA, 23 bp, synthetic construct
  source 1..23: mol_type = other DNA
ttgcttgttcttggacgtataat

SEQ ID NO: 45 - DNA, 23 bp, synthetic construct
  source 1..23: mol_type = other DNA
aacattatacgtccaagaacaag

SEQ ID NO: 46 - DNA, 23 bp, synthetic construct
  source 1..23: mol_type = other DNA
ggagaaccctcttgttattgggg

SEQ ID NO: 47 - DNA, 23 bp, synthetic construct
  source 1..23: mol_type = other DNA
ttgggagaaccctcttgttattg

SEQ ID NO: 48 - DNA, 23 bp, synthetic construct
  source 1..23: mol_type = other DNA
aaccaataacaagagggttctcc

SEQ ID NO: 49 - DNA, 23 bp, synthetic construct
  source 1..23: mol_type = other DNA
aaagtaaataatcatggcacggg

SEQ ID NO: 50 - DNA, 23 bp, synthetic construct
  source 1..23: mol_type = other DNA
ttgaaagtaaataatcatggcac

SEQ ID NO: 51 - DNA, 23 bp, synthetic construct
  source 1..23: mol_type = other DNA
aacgtgccatgattatttacttt

SEQ ID NO: 52 - DNA, 1858 bp, Glycine max
  source 1..1858: mol_type = genomic DNA
ccaaacacaaacatcatagtatatagtatctatctaaagtaaataatcatggcacgggagaaccctcttgttattggtggtgtgattggggatgttctcaacccttttacaagctccgtttctttgactgtttcaatcaataatagggcgattagcaatggcttggaactcaggccctctcaagttgttaatcgccctagggttactgttggtggtgaagacctaaggaccttctacactctggtaagtaattaagtagtatatatagtggaaattaatcaattgctagctagcgagtacaaattagtcttaataaataagttattaacagctagtatgcatgctatatatataaaaatttatggttgaatctcaggttatggtggatgcagatgcacctagccctagcaaccctgtcttgagggaataccttcactggtgaatgagttatctttgcttctgtagtctcccaattgcttctgtttttaatttctattaacaaggaataccttcactgatattgattttttttaaaaggcaaatgaattattattaattaagaagaaaacaggatcaaggtcacaaggagtgtgccacgaaccatgcatcaatattacaaatcatcggggtcaagaaattaaaacatatagctttccataaaagcaggatacaccccgtaaccaagcttatattgattataatttgtatccaaaatttatatagcagtagagcctaaatatgctcaaattttataattttttcgtgaacttgtttattgacatatattatattcgtttgttatcaatctttctctctttaacaatgacaggatggtgacagatattccagctaccacaaatgcaagctttggtaagtacaaatattctaagtgttgtgtatatgcttaagtagccgtgggataaaaataacatgttaaagtttcacatcaattcaaattaaagatatgaccaaaataaattatataagtgagaagtaatcctaatcttacaaatttaagattgagttagatttaaattcactttttataattatatatatatatagacacacatgacatgagacattaatattgatgcacagggagagaggttgtgttttatgagagcccgaacccttcagtagggattcatcgaatcgtgttcgtattgttccagcaattgggcagagacactgtcatcaccccagaatggcgccataatttcaattccagaaactttgctgaaattaataaccttgcacctgttgcagcagcttatgccaactgccaaagagagcgtggttgcggtggaaggagatattaacaagttcaattaatgaagattcttggatgccttactccagctgatactttagagaaataaagacagaagagagaaaaatatatatagtaccttaagtaatatgatgtaataaaaggaaagataaataatgataataataataatgataataataataatagatgcttagtgtaatagaatagtagcactatcatcaaataagttagaagagtaatggagaaaaagaataagttagaagttaataaaatctctccttttttaaataataatttagagagaaggtaccatcttgcaatgctagagcatgccattcaggaacttcttcaacagattatggtagcaacggaaaaaaaaatacaaaaagttcatgcatggttcatacatactacagggatctcatcctgtattattgtaatgtacgaactgttattcattttaaacaacgtttgcttcaataaattacatacaatattattaacattttaccctttcaatttttttatgatgtcctttcttttctggctatccttgtttta

SEQ ID NO: 53 - DNA, 1859 bp, Glycine max
  source 1..1859: mol_type = other DNA
ccaaacacaaacatcatagtatatagtatctatctaaagtaaataatcatgggcacgggagaaccctcttgttattggtggtgtgattggggatgttctcaacccttttacaagctccgtttctttgactgtttcaatcaataatagggcgattagcaatggcttggaactcaggccctctcaagttgttaatcgccctagggttactgttggtggtgaagacctaaggaccttctacactctggtaagtaattaagtagtatatatagtggaaattaatcaattgctagctagcgagtacaaattagtcttaataaataagttattaacagctagtatgcatgctatatatataaaaatttatggttgaatctcaggttatggtggatgcagatgcacctagccctagcaaccctgtcttgagggaataccttcactggtgaatgagttatctttgcttctgtagtctcccaattgcttctgtttttaatttctattaacaaggaataccttcactgatattgattttttttaaaaggcaaatgaattattattaattaagaagaaaacaggatcaaggtcacaaggagtgtgccacgaaccatgcatcaatattacaaatcatcggggtcaagaaattaaaacatatagctttccataaaagcaggatacaccccgtaaccaagcttatattgattataatttgtatccaaaatttatatagcagtagagcctaaatatgctcaaattttataattttttcgtgaacttgtttattgacatatattatattcgtttgttatcaatctttctctctttaacaatgacaggatggtgacagatattccagctaccacaaatgcaagctttggtaagtacaaatattctaagtgttgtgtatatgcttaagtagccgtgggataaaaataacatgttaaagtttcacatcaattcaaattaaagatatgaccaaaataaattatataagtgagaagtaatcctaatcttacaaatttaagattgagttagatttaaattcactttttataattatatatatatatagacacacatgacatgagacattaatattgatgcacagggagagaggttgtgttttatgagagcccgaacccttcagtagggattcatcgaatcgtgttcgtattgttccagcaattgggcagagacactgtcatcaccccagaatggcgccataatttcaattccagaaactttgctgaaattaataaccttgcacctgttgcagcagcttatgccaactgccaaagagagcgtggttgcggtggaaggagatattaacaagttcaattaatgaagattcttggatgccttactccagctgatactttagagaaataaagacagaagagagaaaaatatatatagtaccttaagtaatatgatgtaataaaaggaaagataaataatgataataataataatgataataataataatagatgcttagtgtaatagaatagtagcactatcatcaaataagttagaagagtaatggagaaaaagaataagttagaagttaataaaatctctccttttttaaataataatttagagagaaggtaccatcttgcaatgctagagcatgccattcaggaacttcttcaacagattatggtagcaacggaaaaaaaaatacaaaaagttcatgcatggttcatacatactacagggatctcatcctgtattattgtaatgtacgaactgttattcattttaaacaacgtttgcttcaataaattacatacaatattattaacattttaccctttcaatttttttatgatgtcctttcttttctggctatccttgtttta

SEQ ID NO: 54 - DNA, 2741 bp, Glycine max
  source 1..2741: mol_type = genomic DNA
aacaatatagtatctaacataaagcatggcacgggagaaccctcttgttattgggggtgtgattggggatgttctcaatccttttacaatctccgtttcttttactatttcaatcaataatagggcgattagcaatggcttggaactgaggccctctcaagttgttaatcgccctagagtcactgttggtggtgaagacctaaggaccttctacacactggtaagtagtagtggaaattaactagctatatatagtggatacttgaagtcttaattaacaaataagcagtagtggaaattaactagtgaaattaacagctagttgcatgctatttacataaaaatttatggttgaatatcaggttatggtggatgcagatgcacctagccctagcaaccctgtcttgagggaataccttcactggtgagttatcttgatatgcttctttttttggtatttttgtttatcattcaatgttagttgttagtttttgttcgtgaaagtattcaaatccattaccttctccccctttctttcctttcatcatcaagttaaccatataatctctcttaatttgtttctttctctgaatttctatctgtttttggtagaagcacgggtgaaattctaaaattagcaaaaaatattgatgttaatttgtatttaaagtctatatatagctgtagagcctaaacctaaataagtatataaatgctgctcaaattttataaattttttgtgacttgcttactaatatatattcgtttgttatcaatcttctctctttaacattggcaggatggtgacagatattccagctaccacaaatgcaagctttggtaagtacaaatattctatgtgctgttcataagcttgtatgtgagttgttcttcaagcaagtgttaaaatgggtattgatgtttctatcaatttgaacttcacctgctgcatgcatatcaattgatgaggaagggttagtattattattaatccctcgatcgttaagttattcattttaaattcagtcccattcacgactttatattaatttaattcccgttgatcaaagcttagagattaaggtgtcaatttagaagacattcattggaacaactaataagaaattcattcaaggttaaaaaaagtagactacaatttttgcaactattctaatgatgattaaacttatgttatataaacacttaattaattaagttgtttattcaaatatatctttaaccctcgatcgtgagaagaatttgagctgaggcaaattataactattaaagagctaactgattaacggaagtaaattggatgaatgaatgatatattataaaaaaattattttattggaaagtaaatcagagtaaggataaacatacagtgaaggacttaaatatatctttttctgtcaacaaaactattatttctataattactctgcaactatgtgcctttttatttttattttttggcaaaaccccggtatcatgtgcagttaaaccattgtgcatatcggttcagaaatgtaattgtcaaaaaaagaaaagaaaaagtctgcgtgatgcatgtatgttaagctcgacccctcgtaatgagacgaaatgtataaagattaaacaaactgatacgttttaaatatatagtttgtcaaacagtatgcacaattgcacatgaccatgagtttatccattttcacacttttaataagcatttcggctaaatttcaaatctatgaatatggcaggttaggaaaataacatttttattttttttataaatcacacatatagcaattactttttttgttaattatagtctcttatataatttagaatgtgtaagtgaacacaagaacaataatcccattgtgctttgtgtgtgggtaggtttttatttttttttcatcattgaactaattaacggtattgatcttcaaccaaaaggaacaaattattatatccttgcaaaattagaaattatcaaaaggaattttgtctcttctaacaataagaaaatttcatacactgttgaaaattaaaatctaattcaatatgtctaggagactcgctagttatctgaacaactatatattggactttgtaggttgtgtgtatatatataagattttgcattaacttattattttcttatgtacaataatcattttttttacagaatctagaatatatatatagcaactcaactatttcataaatttatacttatttaaaatattctattttcacacaagatggacataataattacttataacattttatacgccagtttaaccaatcattattttctatatataatttcttgcaaacatatatcttccatttctaattatgctgcttattcactttgtagcacagattgcattttaatgtttatctatacggtgacataaacattggacatatttatgcagggagagaggttgtgttttatgagagcccgaacccttcagcagggattcatcgacttgtgttcatattattccagcaactgggcagagacactgtcatcaccccagaatggcgccataatttcaattccagaaactttgctgaaattaataaccttgcacctgttgcagcagcttatgccaactgccaaagagagcgtggttgcggtggaaggagatattaacaagttcaatgaagatttttttggatgcctctactctagcttatacttagagaaataaagagaaaagtgagagaaaata

SEQ ID NO: 55 - DNA, 2733 bp, Glycine max
  source 1..2733: mol_type = other DNA
aacaatatagtatctaacataaagcatggcacgggagaaccctcgggggtgtgattggggatgttctcaatccttttacaatctccgtttcttttactatttcaatcaataatagggcgattagcaatggcttggaactgaggccctctcaagttgttaatcgccctagagtcactgttggtggtgaagacctaaggaccttctacacactggtaagtagtagtggaaattaactagctatatatagtggatacttgaagtcttaattaacaaataagcagtagtggaaattaactagtgaaattaacagctagttgcatgctatttacataaaaatttatggttgaatatcaggttatggtggatgcagatgcacctagccctagcaaccctgtcttgagggaataccttcactggtgagttatcttgatatgcttctttttttggtatttttgtttatcattcaatgttagttgttagtttttgttcgtgaaagtattcaaatccattaccttctccccctttctttcctttcatcatcaagttaaccatataatctctcttaatttgtttctttctctgaatttctatctgtttttggtagaagcacgggtgaaattctaaaattagcaaaaaatattgatgttaatttgtatttaaagtctatatatagctgtagagcctaaacctaaataagtatataaatgctgctcaaattttataaattttttgtgacttgcttactaatatatattcgtttgttatcaatcttctctctttaacattggcaggatggtgacagatattccagctaccacaaatgcaagctttggtaagtacaaatattctatgtgctgttcataagcttgtatgtgagttgttcttcaagcaagtgttaaaatgggtattgatgtttctatcaatttgaacttcacctgctgcatgcatatcaattgatgaggaagggttagtattattattaatccctcgatcgttaagttattcattttaaattcagtcccattcacgactttatattaatttaattcccgttgatcaaagcttagagattaaggtgtcaatttagaagacattcattggaacaactaataagaaattcattcaaggttaaaaaaagtagactacaatttttgcaactattctaatgatgattaaacttatgttatataaacacttaattaattaagttgtttattcaaatatatctttaaccctcgatcgtgagaagaatttgagctgaggcaaattataactattaaagagctaactgattaacggaagtaaattggatgaatgaatgatatattataaaaaaattattttattggaaagtaaatcagagtaaggataaacatacagtgaaggacttaaatatatctttttctgtcaacaaaactattatttctataattactctgcaactatgtgcctttttatttttattttttggcaaaaccccggtatcatgtgcagttaaaccattgtgcatatcggttcagaaatgtaattgtcaaaaaaagaaaagaaaaagtctgcgtgatgcatgtatgttaagctcgacccctcgtaatgagacgaaatgtataaagattaaacaaactgatacgttttaaatatatagtttgtcaaacagtatgcacaattgcacatgaccatgagtttatccattttcacacttttaataagcatttcggctaaatttcaaatctatgaatatggcaggttaggaaaataacatttttattttttttataaatcacacatatagcaattactttttttgttaattatagtctcttatataatttagaatgtgtaagtgaacacaagaacaataatcccattgtgctttgtgtgtgggtaggtttttatttttttttcatcattgaactaattaacggtattgatcttcaaccaaaaggaacaaattattatatccttgcaaaattagaaattatcaaaaggaattttgtctcttctaacaataagaaaatttcatacactgttgaaaattaaaatctaattcaatatgtctaggagactcgctagttatctgaacaactatatattggactttgtaggttgtgtgtatatatataagattttgcattaacttattattttcttatgtacaataatcattttttttacagaatctagaatatatatatagcaactcaactatttcataaatttatacttatttaaaatattctattttcacacaagatggacataataattacttataacattttatacgccagtttaaccaatcattattttctatatataatttcttgcaaacatatatcttccatttctaattatgctgcttattcactttgtagcacagattgcattttaatgtttatctatacggtgacataaacattggacatatttatgcagggagagaggttgtgttttatgagagcccgaacccttcagcagggattcatcgacttgtgttcatattattccagcaactgggcagagacactgtcatcaccccagaatggcgccataatttcaattccagaaactttgctgaaattaataaccttgcacctgttgcagcagcttatgccaactgccaaagagagcgtggttgcggtggaaggagatattaacaagttcaatgaagatttttttggatgcctctactctagcttatacttagagaaataaagagaaaagtgagagaaaata

SEQ ID NO: 56 - DNA, 528 bp, Glycine max
  source 1..528: mol_type = other DNA
atgcctatatcaatggacccccttgttcttggacgtataataggagatattttggatcccttcacgagttctgtttctctgagggttgtttataacaaccagtcatcggttatcaatagttgtgagttcaaaccttcccaaatcgtcaacaaaccaagaattaatattcgtggaaatgacctagggatcttttacactctgataatggtgaaccccgatgctcctagcccaagtgacccacatatgaaggaatatttgcattggttggtaaccaatattccagcatctacaggggcaaccactggagaagagattgtggagtatgaaagtccacgaccaacttctgggattcatcggattgcttttgtgttgtttcgtcaatttgatagacaaattgtgcatgctccaagatggcgccaaaatttcaacactagagattttgctgaggtttacaaccttggatcaccagttgctgctgtgtatttcaactgtcaacgtgaaggtggttggggtggacgaaggagatga

SEQ ID NO: 57 - DNA, 523 bp, Glycine max
  source 1..523: mol_type = other DNA
atgcctatatcaatggacccccttgttcttggaaataggagatattttggatcccttcacgagttctgtttctctgagggttgtttataacaaccagtcatcggttatcaatagttgtgagttcaaaccttcccaaatcgtcaacaaaccaagaattaatattcgtggaaatgacctagggatcttttacactctgataatggtgaaccccgatgctcctagcccaagtgacccacatatgaaggaatatttgcattggttggtaaccaatattccagcatctacaggggcaaccactggagaagagattgtggagtatgaaagtccacgaccaacttctgggattcatcggattgcttttgtgttgtttcgtcaatttgatagacaaattgtgcatgctccaagatggcgccaaaatttcaacactagagattttgctgaggtttacaaccttggatcaccagttgctgctgtgtatttcaactgtcaacgtgaaggtggttggggtggacgaaggagatga

SEQ ID NO: 58 - DNA, 529 bp, Glycine max
  source 1..529: mol_type = other DNA
atgcctatatcaatggacccccttgttcttggacgtattaataggagatattttggatcccttcacgagttctgtttctctgagggttgtttataacaaccagtcatcggttatcaatagttgtgagttcaaaccttcccaaatcgtcaacaaaccaagaattaatattcgtggaaatgacctagggatcttttacactctgataatggtgaaccccgatgctcctagcccaagtgacccacatatgaaggaatatttgcattggttggtaaccaatattccagcatctacaggggcaaccactggagaagagattgtggagtatgaaagtccacgaccaacttctgggattcatcggattgcttttgtgttgtttcgtcaatttgatagacaaattgtgcatgctccaagatggcgccaaaatttcaacactagagattttgctgaggtttacaaccttggatcaccagttgctgctgtgtatttcaactgtcaacgtgaaggtggttggggtggacgaaggagatga

SEQ ID NO: 59 - DNA, 2670 bp, Glycine max
  source 1..2670: mol_type = other DNA
acagccaataagattacatgaaaggaaagcatatatattaagggataacatgagattttgactggtgcatgtatatagaatctacatggacccaaaattacacgtgtctccaagttgtgcctccatagcaatacgaacaaagcctcaacatctctttaatgattccaaaaggcaaaggaagcaacatgcacaggtccaacaaaaaaaaaaaaaaaaaacctctctcctattcaaccccaaaacacaaccaaccacaagataagcaagaacttgtgcacgccactaacttgctgccacgatggcacccttgattctcccccaacactacttggttctcctcactgaactcaacttctcactcacactcacttctcttcctccaaattagttccaactccaagttagttccaacacaaacacaaacacacacacatacataaaccaagcaaagaaagactctacaaacacttcactactcatacaatattttcagacacaacatgttgaaggaaggcaccaacaacgttggtggcaacctggtcacatgtctgcgacacgtgccggtcagcgccatgcgtactgtactgccatgcagactcagcatacctttgctcatcctgcgatgctcgtgtccatgcggctaaccgtgtggcctcaagacacgagcgtgtgtgggtgtgcgaagcgtgtgaacgtgctcccgcagcgtttctatgcaaagccgacgcagcttctctttgttcttcctgtgatgctgacattcactcagcaaaccctctcgctagccgccaccaccgcgtgcccattctcccgatctctggttccctcttcggggaaccagagcatgaacgcgtgtacgcgttcgtgaatgaagtggaagcggaggaggaagaggaagaggtttttgatgagtatgatgaggttgaagcagcttcgtggttgttgccacatcctatgaaaaatgataaaattgacgagaatggtggtgataagggttttttgtttggtgatgagtatttcgacaaccttgttgattgtaactcatgtggtcacaataacaaccagtttagcaacgtttatgatcagcaccagcagaattacagcaacactgtccctcagaactatgcagtggttccagttcaggtgccgcagcattttcaaccgggtttggactttgactcatcaaaagctgggttcagttacgatggttctcttagtcaaagtgtaagttaccttctttttacttctatgtgtttctttttggtttgttgtttatatcaatggggttttcccttctttttttttttttttttctttttgcaaactctttggatcattcatccattggattatactactttattagattttgccttgaaacctgtgctaactcttccttctaattgacagtttctttttctccattcgtatgaaaccaaggttttaaactgcagtcatagtcatagtcaagattatagtttattcaaaaacattagcattgtaatacaattgtactgtaattgtcgtcgtgaaaaagttaaaaaaaagtttaaaaaatctcaatcgcataccttctataaaaatcttgattgaaaccaaagtgggtttcataagtagtttcttttacaagaaaaactagcaataaagtcaactactctagatccattgccatacatgtgaccttgatccattccacaacgtttcgtatttctttgcctacccttttcccattgactacttttctgtcttgagagacatttattttatttttttcgtatgcaatagctttccctttttgatgtcttgtatttgctagttatagttgtgattcctcataaacagatacttacttttatagggtaacttagcttttcaccaattttcgcccatacatagttagacttccctaaactttgtttcctcatttcttcaactataatttgatcaattgtatattaatttcccataattttcagcttcaattaaaaaatttgagcctaagtgcctgttttattaaagccaaacaatggggggtttaatattttaaaaactttcttctatatttcttttacatttcctctgttttctaatatatcttttcttttttctattacatcatctttgatatattttcacctttttcttcttactttggggacaaacattattgtttcacaaaattggttagctactaatagtttattgccttacctgtaatctctcaaacttcatacacgtttttgtgttaatcttttatatttcatttttaatttaggtttcggtttcatcgatggatgttggtgttgtactcgaatcaacaataagtgacatctcaatgtcccactcaaagtcgccaatagggacaactgacctatttcctccccttcccatgccttcacatctcacaccaatggacagagaggcaagagtcctaagatacagggagaaaaagaagacaagaaaatttgagaagaaaataaggtatgcctcaaggaaggcctatgcagagactagaccccgcataaagggtcgttttgcaaagagaaccgatgtagaagctgaagtggaccagatgttctccacaacactattcactgaagttggaggtagcatttttcccactttctagaatggaaagaaagtaatggccaggccataaaagagaaggtt

SEQ ID NO: 60 - DNA, 1078 bp, Glycine max
  source 1..1078: mol_type = other DNA
atgttgaaggaaggcaccaacaacgttggtggcaacctggtcacatgtctgcgacacgtgccggtcagcgccatgcgtactgtactgccatgcagactcagcatacctttgctcatcctgcgatgctcgtgtccatgcggctaaccgtgtggcctcaagacacgagcgtgtgtgggtgtgcgaagcgtgtgaacgtgctcccgcagcgtttctatgcaaagccgacgcagcttctctttgttcttcctgtgatgctgacattcactcagcaaaccctctcgctagccgccaccaccgcgtgcccattctcccgatctctggttccctcttcggggaaccagagcatgaacgcgtgtacgcgttcgtgaatgaagtggaagcggaggaggaagaggaagaggtttttgatgagtatgatgaggttgaagcagcttcgtggttgttgccacatcctatgaaaaatgataaaattgacgagaatggtggtgataagggttttttgtttggtgatgagtatttcgacaaccttgttgattgtaactcatgtggtcacaataacaaccagtttagcaacgtttatgatcagcaccagcagaattacagcaacactgtccctcagaactatgcagtggttccagttcaggtgccgcagcattttcaaccgggtttggactttgactcatcaaaagctgggttcagttacgatggttctcttagtcaaagtgtttcggtttcatcgatggatgttggtgttgtactcgaatcaacaataagtgacatctcaatgtcccactcaaagtcgccaatagggacaactgacctatttcctccccttcccatgccttcacatctcacaccaatggacagagaggcaagagtcctaagatacagggagaaaaagaagacaagaaaatttgagaagaaaataaggtatgcctcaaggaaggcctatgcagagactagaccccgcataaagggtcgttttgcaaagagaaccgatgtagaagctgaagtggaccagatgttctccacaacactattcactgaagttggaggtagcatttttcccactttctag

SEQ ID NO: 61 - protein, 47 aa, Glycine max
MLKEGTNNVGGNLVTCLRHVPVSAMRTVLPCRLSIPLLILRCSCPCG

SEQ ID NO: 62 - DNA, 2707 bp, Glycine max
  source 1..2707: mol_type = other DNA
accaaacacttgctttaatccttttagagccaataagattactgagaggaaaggatatattaaggggtaacaagagattctaactggtgcaagtatagaatctacatggacccaaatgacacgtgtctccaagttgtgtcaccatagcaatacgaacaaagcctcagcatctctttaatgattccaaaaggcaaagaagcaacatgcatggacaggtccaacaccaaaacaaaaaaccctctctcctaatcaaccctgaaacacaaccaaccacaagataagcaagaacttgtgcacgccactaacttgctgccacgttggcactcttgattctccccccaacactacttggttcacctcactaaactcaatttctcactcacactcacttcagttcaattcacttcccttcctccaaattagttccaacacaaaaaccaagcaaagaaaaagactctaccgaaacacttcactactcatacaatattttcagacacaacatgttgaaggaaggcaccaacaacgttggtggcagcaacactggcacctggtcacgtgtctgtgacacgtgcctgtctgcgccatgcgtgctgtactgccatgcagactcagcatatctttgctcttcctgtgatgctcgtgtgcatgcagctaaccgtgtggcctcgagacacaagcgtgtgtgggtgtgcgaagcgtgtgagcgtgctccggcggcgtttctatgcaaagctgacgcagcttctctttgttcttcctgtgatgctgacattcactcagcaaaccctctcgctagccgccacaaccgtgttcccattctcccgatctcaggttccctcttcagggaaccagagcacaatcacaaacgcgtggaacacgcgttcgtgaatgaggttgaggaggaagaggaaggggtttttgatgagtatgaagacgaggttgaagctgcttcgtggttgttgccacatcctatgaaaaataatgatgaaattgaggagaatgattgtggtgacgagggttttttgtttgttgatgagtatttggacaatcttgttgattgttgtaactcatgtggtcacaatgacaaccagtttagcaacgtttatcagcaccagcagaattacaacactgtccctcagaactatgtagtggttccagttcaggtgccgcagcattttcaacccggtttggactttgactcatcaaaagctgggttcagttacgatggttctcttagtcaaagtgtaagactcttcttttcaattacttctgtgtttctttttcggtttgttttttatatgaatagtgttttccctttttctctttctttttcttttcccagactctttggatcattcatccattggattatactttatcagattttgattttgccttgacacctttgctaactcttccttccaattggcagtttctttcctttctccgttcatatgaaactagggttttaaattatactcatgatcaataatttggttgcaatttttgatattgtagtacaattgctattgttgttgtgaaaagttaaaaaaatcttaatgttgtaaccgaaatctcaatcacatactgtttgtaaaatccttggttgaaatcaaagtgggtttcccaagtagcttcttttactagaaacgctagcaaagtcaactactctagttcaattgccatatatgtgactaatttgattcattccacaactcttcatatttctttgactacccttttaccgttcactttgcggtcttgagagacttttatttattttcttttttgtcgtatataagatcagctttgcctttatggtgtcttttatttgctagttatagttgtgattcctcttaaactttgtttcctcatttcatcaactgtatattaatttcccataatttttcagcttcaattaaaaaaattgagcctaagtgcctgatttattaaagccacaaaatgggtggtttaattttttcgacacctttcttttatatttcttttactcttcttctcttttctttttctattacatcatctataatatatcttcacctttttcttcttactttggggacaaacatttttgtttcacaaagctggttagctactagtttattgcttagaaaatttatagccttacctgtgatctctcaaacttcatacacgattttgtgttaatcttttatatttcatttttaatttaggtttcggtttcatcaatggatgttggagttgtacccgaatcaacagtaagtggcatctcaatgtcccactcaaagtcaccaatagggacaaatgacctatttcctccccttctcatgccttcacatctcacaccaatggacagagaggcaagagtcctaagatatagggagaaaaagaagacaagaaagttcgagaagaaaataagatatgcctcaaggaaggcctatgcagagactagaccccgcataaagggtcgttttgcaaagagaactgatgtagaagctgaagtggatcagatgttctccacaaaactattcaatgaagttggaggtagcatttttcccactttctagaatggaaagaaagtaattgccaagccatagaagagaaggttgggaaccacacatcattggaagaacttcatgtaatttttatgtagtctaattagtttggtattatatttacttctatcttgtgctttataaatataaagtat

SEQ ID NO: 63 - DNA, 1098 bp, Glycine max
  source 1..1098: mol_type = other DNA
atgttgaaggaaggcaccaacaacgttggtggcagcaacactggcacctggtcacgtgtctgtgacacgtgcctgtctgcgccatgcgtgctgtactgccatgcagactcagcatatctttgctcttcctgtgatgctcgtgtgcatgcagctaaccgtgtggcctcgagacacaagcgtgtgtgggtgtgcgaagcgtgtgagcgtgctccggcggcgtttctatgcaaagctgacgcagcttctctttgttcttcctgtgatgctgacattcactcagcaaaccctctcgctagccgccacaaccgtgttcccattctcccgatctcaggttccctcttcagggaaccagagcacaatcacaaacgcgtggaacacgcgttcgtgaatgaggttgaggaggaagaggaaggggtttttgatgagtatgaagacgaggttgaagctgcttcgtggttgttgccacatcctatgaaaaataatgatgaaattgaggagaatgattgtggtgacgagggttttttgtttgttgatgagtatttggacaatcttgttgattgttgtaactcatgtggtcacaatgacaaccagtttagcaacgtttatcagcaccagcagaattacaacactgtccctcagaactatgtagtggttccagttcaggtgccgcagcattttcaacccggtttggactttgactcatcaaaagctgggttcagttacgatggttctcttagtcaaagtgtttcggtttcatcaatggatgttggagttgtacccgaatcaacagtaagtggcatctcaatgtcccactcaaagtcaccaatagggacaaatgacctatttcctccccttctcatgccttcacatctcacaccaatggacagagaggcaagagtcctaagatatagggagaaaaagaagacaagaaagttcgagaagaaaataagatatgcctcaaggaaggcctatgcagagactagaccccgcataaagggtcgttttgcaaagagaactgatgtagaagctgaagtggatcagatgttctccacaaaactattcaatgaagttggaggtagcatttttcccactttctag

SEQ ID NO: 64 - protein, 365 aa, Glycine max
MLKEGTNNVGGSNTGTWSRVCDTCLSAPCVLYCHADSAYLCSSCDARVHAANRVASRHKRVWVCEACERAPAAFLCKADAASLCSSCDADIHSANPLASRHNRVPILPISGSLFREPEHNHKRVEHAFVNEVEEEEEGVFDEYEDEVEAASWLLPHPMKNNDEIEENDCGDEGFLFVDEYLDNLVDCCNSCGHNDNQFSNVYQHQQNYNTVPQNYVVVPVQVPQHFQPGLDFDSSKAGFSYDGSLSQSVSVSSMDVGVVPESTVSGISMSHSKSPIGTNDLFPPLLMPSHLTPMDREARVLRYREKKKTRKFEKKIRYASRKAYAETRPRIKGRFAKRTDVEAEVDQMFSTKLFNEVGGSIFPTF

SEQ ID NO: 65 - DNA, 24 bp, synthetic construct
  source 1..24: mol_type = other DNA
cgccagggttttcccagtcacgac

SEQ ID NO: 66 - DNA, 24 bp, synthetic construct
  source 1..24: mol_type = other DNA
tgggaatctgaaagaagagaagca

SEQ ID NO: 67 - DNA, 25 bp, synthetic construct
  source 1..25: mol_type = other DNA
tgaagtggacggaaggaggaggagg

SEQ ID NO: 68 - DNA, 20 bp, synthetic construct
  source 1..20: mol_type = other DNA
tcacacgcgcaagaacgtat

SEQ ID NO: 69 - DNA, 20 bp, synthetic construct
  source 1..20: mol_type = other DNA
ctaggagcatcggggttcac

SEQ ID NO: 70 - DNA, 24 bp, synthetic construct
  source 1..24: mol_type = other DNA
gatgaagtggacggaaggaaggag

SEQ ID NO: 71 - DNA, 20 bp, synthetic construct
  source 1..20: mol_type = other DNA
ttgaccatgcaccaagggaa

SEQ ID NO: 72 - DNA, 20 bp, synthetic construct
  source 1..20: mol_type = other DNA
caagacagggttgctagggc

SEQ ID NO: 73 - DNA, 20 bp, synthetic construct
  source 1..20: mol_type = other DNA
atcgaccgatcgaggacaac

SEQ ID NO: 74 - DNA, 22 bp, synthetic construct
  source 1..22: mol_type = other DNA
tgggagactacagaagcaaaga

SEQ ID NO: 75 - DNA, 35 bp, synthetic construct
  source 1..35: mol_type = other DNA
aacaacgttggtggcagcaccggcacctggtcaca

SEQ ID NO: 76 - DNA, 33 bp, synthetic construct
  source 1..33: mol_type = other DNA
caacgttggtggcagcaccggcacctggtcaca

SEQ ID NO: 77 - DNA, 25 bp, synthetic construct
  source 1..25: mol_type = other DNA
caacgttggtggcaacctggtcaca

SEQ ID NO: 78 - DNA, 25 bp, synthetic construct
  source 1..25: mol_type = other DNA
aggcaaaggaagcatgcgtactgta

SEQ ID NO: 79 - DNA, 40 bp, synthetic construct
  source 1..40: mol_type = other DNA
caacgttggtggcagcaacactggcaccacctggtcacgt

SEQ ID NO: 80 - DNA, 33 bp, synthetic construct
  source 1..33: mol_type = other DNA
tggtggcagcaacactggcaccacctggtcacg

SEQ ID NO: 81 - DNA, 32 bp, synthetic construct
  source 1..32: mol_type = other DNA
tggtggcagcaacactggcacacctggtcacg

SEQ ID NO: 82 - DNA, 30 bp, synthetic construct
  source 1..30: mol_type = other DNA
tggtggcagcaacactggcacctggtcacg

SEQ ID NO: 83 - DNA, 69 bp, synthetic construct
  source 1..69: mol_type = other DNA
atgcctatatcaatggacccccttgttcttggacgtataataggagatattttggatcccttcacgagt

SEQ ID NO: 84 - DNA, 69 bp, synthetic construct
  source 1..69: mol_type = other DNA
tctgtttctctgagggttgtttataacaaccagtcatcggttatcaatagttgtgagttcaaaccttcc

SEQ ID NO: 85 - DNA, 63 bp, synthetic construct
  source 1..63: mol_type = other DNA
caaatcgtcaacaaaccaagaattaatattcgtggaaatgacctagggatcttttacactctg

SEQ ID NO: 86 - DNA, 33 bp, synthetic construct
  source 1..33: mol_type = other DNA
ccccttgttcttggacgtataataggagatatt

SEQ ID NO: 87 - DNA, 28 bp, synthetic construct
  source 1..28: mol_type = other DNA
ccccttgttcttggaaataggagatatt

SEQ ID NO: 88 - DNA, 34 bp, synthetic construct
  source 1..34: mol_type = other DNA
ccccttgttcttggacgtattaataggagatatt

SEQ ID NO: 89 - DNA, 35 bp, synthetic construct
  source 1..35: mol_type = other DNA
atggcacgggagaaccctcttgttattgggggtgt

SEQ ID NO: 90 - DNA, 36 bp, synthetic construct
  source 1..36: mol_type = other DNA
gattggggatgttctcaatccttttacaatctccgt

SEQ ID NO: 91 - DNA, 37 bp, synthetic construct
  source 1..37: mol_type = other DNA
ctatctaaagtaaataatcatggcacgggagaaccct

SEQ ID NO: 92 - DNA, 36 bp, synthetic construct
  source 1..36: mol_type = other DNA
cttgttattggtggtgtgattggggatgttctcaac

(19) United States
(12) Patent Application Publication
(10) Pub. No.: US 2026/0201403 A1
(43) Pub. Date: Jul. 16, 2026

(54) GENES ALTERING SOY PLANT FLOWERING TIME AND/OR MATURATION AND USES THEREOF

(71) Applicants: INSTITUTE OF CROP SCIENCES, CHINESE ACADEMY OF AGRICULTURAL SCIENCES, Beijing (CN); SYNGENTA CROP PROTECTION AG, BASEL (CH)

(72) Inventors: Wensheng HOU, Beijing (CN); Yupeng CAI, Beijing (CN); Li CHEN, Beijing (CN); Shan YUAN, Beijing (CN); Qiang SU, Beijing (CN); Shi SUN, Beijing (CN); Tianfu HAN, Beijing (CN); Yang GAO, Beijing (CN)

(73) Assignees: INSTITUTE OF CROP SCIENCES, CHINESE ACADEMY OF AGRICULTURAL SCIENCES, Beijing (CN); SYNGENTA CROP PROTECTION AG, BASEL (CH)

(21) Appl. No.: 19/127,958

(22) PCT Filed: Oct. 31, 2023
(86) PCT No.: PCT/US2023/036482

§ 371 Date: May 7, 2025

(30) Foreign Application Priority Data
Nov. 7, 2022 (WO) PCT/CN2022/130366

(51) Int. Cl. C12N 15/82; C07K 14/415; C12N 9/22; C12N 15/11

(52) U.S. Cl. CPC C12N 15/827; C07K 14/415; C12N 9/226; C12N 15/111; C12N 15/8201; C12N 2310/20

(57) ABSTRACT

Compositions and methods for altering flowering and/or maturity time of soybean plant are provided. Compositions include isolated and recombinant polynucleotides encoding polypeptides, expression cassettes, host cells, plants, plant parts stably incorporating these polynucleotides. Methods and kits are provided for producing these plants via transgenic means, breeding or genomic editing approaches and identify plants having altered flowering and/or maturity time.

### RELATED APPLICATION

This application claims the benefit of and priority to International Application No. PCT/CN2022/130366, filed on Nov. 7, 2022. The entire content of said application is herein incorporated by reference for all purposes.

### FIELD

This disclosure relates to the field of plant biotechnology. In particular, it relates to genes, and methods and compositions for use thereof, in altering the flowering time and/or maturity time of photoperiodic plants to enable them to be cultivated in a variety of geographical locations having different day lengths.

### SEQUENCE LISTING

The official copy of the sequence listing is submitted herewith as an XML formatted sequence listing with a file named 109098-1412651.xml, created on Oct. 25, 2023, and having a size of 146,163 bytes, and is filed concurrently with the specification. The sequence listing contained in this xml formatted document is part of the specification and is herein incorporated by reference in its entirety.

### BACKGROUND

The world population is expected to reach 9.3 billion by 2050, according to the United Nations, and the production of sufficient protein and oil for consumption by humans and livestock poses a significant challenge given increasingly limited resources. Soybean is a valuable field crop that humans rely on for food. Soybean oil extracted from the seed is widely used in cooking oil, baked goods, margarines and the like. Soybean meal and sour flour are components of many foods and animal feed. Soybean proteins also offer a healthier and less expensive replacement for animal protein in meats as well as dairy-type products.

Most flowering plants respond to daily photoperiodic cycles and are classified as either short day (SD) or long day (LD) plants based on the photoperiodic conditions required to induce flowering. Photoperiodic conditions experienced by a plant, in turn, are a function of the geographical location (e.g., latitude or longitude) where they are cultivated. Soybeans are short-day (SD) plants requiring days to be shorter than a critical value to induce flowering. Soybean varieties are classified into maturity groups according to their response to the photoperiod, such as based on the number of days till flowering occurs. For example, with a typical planting date of May 1st for most North American soy varieties, the vegetative period of soybean growth can last from 55-65 days with flowering beginning around mid-July. Given that soybean flowering and maturity time are sensitive to seasonal changes in day length (i.e., photoperiod), soybean cultivation is limited by certain geographical ranges. Thus, there is a need to produce soybean plants with altered flowering time and/or maturity time to enable the crop to be cultivated in a wide range of geographical locations.

### BRIEF SUMMARY

In one aspect, disclosed herein is a plant having a genomic modification, wherein the genomic modification comprises a knock out of one or more of the following genes: GmCOL2a; GmCOL2b; GmFT5a; GmFT5b; or GmFT4, wherein the plant has an altered flowering time and/or maturity time relative to a control plant not comprising the genomic modification.

In another aspect, disclosed herein is a plant expressing both a mutant GmCOL2a polypeptide and a mutant GmCOL2b polypeptide, wherein mutant GmCOL2a polypeptide comprises: (a) an amino acid sequence that is at least 85% identical to SEQ ID NO: 20, or (b) an amino acid sequence as set forth in SEQ ID NO: 20, and wherein the mutant GmCOL2b polypeptide comprises an amino acid sequence that has at least 85% identity to SEQ ID NO: 26, or (b) an amino acid sequence as set forth in SEQ ID NO: 26.

In another aspect, disclosed herein is a plant expressing both a mutant GmFT5a polypeptide and a mutant GmFT5b polypeptide, wherein mutant GmFT5a polypeptide comprises: (a) an amino acid sequence that is at least 85% identical to SEQ ID NO: 42, or (b) an amino acid sequence as set forth in SEQ ID NO: 42, and wherein the mutant GmFT5b polypeptide comprises an amino acid sequence that has at least 85% identity to SEQ ID NO: 38, or (b) an amino acid sequence as set forth in SEQ ID NO: 38.

In another aspect, disclosed herein is a method of altering flowering time and/or maturity time in a soybean plant, the method comprising, editing in the genome of a soybean plant one or more of the following genes: GmCOL2a GmCOL2b, GmFT5a, GmFT5b, or GmFT4, thereby forming a modified soybean plant, wherein the modified soybean plant has a flowering time and/or maturity time that is altered relative to a control plant not comprising the editing in one or more of the genes.

In another aspect, disclosed herein is a plant comprising a genomic modification that results in decreased expression and/or activity of a polypeptide comprising: (a) an amino acid sequence comprising at least 85%, 90%, 91%, 92%, 93%, 94%, 95%, 96%, 97%, 98%, or 99% identity to at least one of SEQ ID NOS: 7, 23, 28, 36, or 40, or (b) an amino acid sequence as set forth in at least one of SEQ ID NOS: 17, 23, 28, 36, or 40, wherein the modification is heterologous to the plant and the decreased expression and/or activity in the plant results in the plant having an altered flowering and/or maturity time compared to a control plant not comprising the genomic modification, and wherein the genomic modification is introduced via genome editing.

In another aspect, disclosed herein is a modified soybean plant, or plant part thereof, comprising one or more non-naturally occurring mutant alleles at one or more loci, wherein the non-naturally occurring mutant allele is introduced via genomic modification using a site directed nuclease, wherein the one or more loci comprise GmFT4a, GmFT5a, GmFT5b, GmCOL2a, or GmCOL2b, and wherein the one or more mutant alleles result in an altered flowering and/or maturity time of the plant relative to a control plant not comprising the mutant allele.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows the gene structures of GmCOL2a with target sites therein for gene editing using e.g., CRISPR/Cas9 according to certain aspects of this disclosure. The grey bars represent the location of the exon, the black line represents the location of the intron, and the end cap region on the right represents the location of the untranslated regions (UTR). The target sequence is named COL2a-SP1. Dashes represent deletions. The arrowheads indicate the location of mutations. PAM: protospacer adjacent motif FIG. 1 contains SEQ ID NO: 75-78 from top to bottom.

FIG. 2 compares the flowering time of WT plants (left) and GmCOL2a mutant plants (right) under SD and LD conditions (SD 22 DAE, top image; LD 34 DAE, bottom image) according to certain aspects of this disclosure. Magnified views of the content within the two boxes in the bottom panel are shown on the upper left and upper right corners of the panel. DAE, days after emergence. Histogram of flowering time, the flowering time values are shown as the mean±one standard deviation. **, P<0.01.

FIG. 3 shows gene structures of GmCOL2b with target sites therein for gene editing using CRISPR/Cas9 according to certain aspects of this disclosure. The grey bars represent the location of the exon, the black line represents the location of the intron, and the end cap region on the right represents the location of the untranslated regions (UTR). The target sequence is named COL2b-SP1. PAM, protospacer adjacent motif. Dashes represent deletions. The red arrowheads indicate the location of mutations. FIG. 3 contains SEQ ID NO: 79-82 from top to bottom.

FIG. 4 compares the flowering time of WT plants (left) and Gmcol2b mutant plants (right) under SD and LD conditions (SD 22 DAE, top image; LD 32 DAE, bottom image) according to certain aspects of this disclosure. Magnified views of the content within the two boxes in the bottom panel are shown on the upper left and upper right corners of the panel. DAE stands for “days after emergence”. The flowering time values are shown as the mean±one standard deviation. **, P<0.01.

FIG. 5 compares the flowering time of WT plants (left) and Gmcol2a Gmcol2b double mutant plants (right) under SD and LD conditions (SD 22 DAE, top image; LD 22 DAE, bottom image) according to certain aspects of this disclosure. Magnified views of the content within the two boxes in the bottom panel are shown on the upper left and upper right corners of the panel. DAE stands for “days after emergence”. The flowering time values are shown as the mean±one standard deviation. **, P<0.01.

FIG. 6A compares the target site of genome editing in the GmFT4 locus according to certain aspects of this disclosure. The underlined sequence is the target site. FIG. 6A contains SEQ ID NO:83 (top), SEQ ID NO:84 (middle), and SEQ ID NO:85 (bottom).

FIG. 6B shows homozygous targeted mutagenesis of GnFT4 induced by gene editing, e.g., CRISPR/Cas9 according to certain aspects of this disclosure. Sequences of wild type and exemplary mutation types induced at target sites GnFT4 are presented. Dashes represent deletions and underlines represent insertions. The arrowheads indicate the location of mutations. FIG. 6B contains SEQ ID NO: 86-88, in the order of top left, top right, and bottom.

FIG. 7 shows the structure of GmFT5b gene and target sites therein for gene editing using gene editing, e.g., CRISPR/Cas9 according to certain aspects of this disclosure. GmFT5b has four exons and three introns, which are represented by the black bands and lines, respectively. The grey bands on the left and right represent untranslated regions. The PAM region (protospacer adjacent motif) is GGG. The remainder of the underlined sequence is the target site sequence recognized by gene editing, e.g., CRISPR/Cas9. FIG. 7 contains SEQ ID NO:89 (top) and SEQ ID NO:90 (bottom).

FIG. 8 shows the flowering phenotypes of WT plants (left) and Gmft5b mutant plants (right) under SD conditions (SD 26 days DAE) according to certain aspects of this disclosure. The top images show magnified regions of the plants in the bottom image. DAE, days after emergence. The values of flowering time, R7 time, plant height and node number are shown as the mean±one standard deviation. **, P<0.01. The bar represents 30 cm.

FIG. 9 shows the flowering phenotypes of WT plants (left) and Gmft5b mutant plants (right) under LD conditions (LD 45 days DAE) according to certain aspects of this disclosure. The top images show magnified regions of the plants in the bottom image. DAE, days after emergence. The values of flowering time, R7 time, plant height and node number are shown as the mean±one standard deviation. **, P<0.01. The bar represents 30 cm.

FIG. 10 shows the structure of GmFT5a gene and target sites for gene editing, e.g., CRISPR/Cas9 target sites according to certain aspects of this disclosure. The green bands represent untranslated regions. GnFT5a has four exons and three introns, which are represented by the black bands and lines, respectively. The grey bands on the left and right represent untranslated regions. The PAM region (protospacer adjacent motif) is GGG. The remainder of the underlined sequence is the target site sequence for gene editing, e.g., CRISPR/Cas9 target sites. FIG. 10 contains SEQ ID NO: 91-92 from top to bottom.

FIG. 11 shows the flowering phenotypes of WT plants (left) and Gmft5a mutant plants (right) under SD conditions (SD 26 days DAE) according to certain aspects of this disclosure. The top images show magnified regions of the plants in the bottom image. DAE, days after emergence. The values of flowering time, R7 time, plant height and node number are shown as the mean±one standard deviation. **, P<0.01. The bar represents 30 cm.

FIG. 12 shows the flowering phenotypes of WT plants (left) and Gmft5a mutant plants (right) under LD conditions (LD 45 days DAE) according to certain aspects of this disclosure. The top images show magnified regions of the plants in the bottom image. DAE, days after emergence. The values of flowering time, R7 time, plant height and node number are shown as the mean±one standard deviation. **, P<0.01. Bar, The bar represents 30 cm.

FIG. 13 shows the flowering phenotypes of WT plants (left) and Gmft5a Gmft5b double mutant plants under SD conditions (SD 26 days DAE) according to certain aspects of this disclosure. The top images show magnified regions of the plants in the bottom image. DAE, days after emergence. The values of flowering time, R7 time, plant height and node number are shown as the mean±one standard deviation. **, P<0.01. The bar represents 30 cm.

FIG. 14 shows the flowering phenotypes of WT plants (left) and Gmft5a Gmft5b double mutant plants under LD conditions (LD 75 days DAE) according to certain aspects of this disclosure. The top images show magnified regions of the plants in the bottom image. DAE, days after emergence. The values of flowering time, plant height and R7 time are shown as the mean±one standard deviation. **, P<0.01. The bar represents 30 cm.

FIG. 15 shows the phenotypes of soybean plants at various vegetative and reproductive stages according to certain aspects of this disclosure.

### DETAILED DESCRIPTION

### I. Terminology

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. Gene names referenced in this disclosure are italicized, while normal text is used for the corresponding protein.

Unless otherwise stated, identity and similarity will be calculated by the Needleman-Wunsch global alignment and scoring algorithms (Needleman and Wunsch (1970) J. Mol. Biol. 48(3):443-453) as implemented by the “needle” program, distributed as part of the EMBOSS software package (Rice, P., Longden, I., and Bleasby, A., EMBOSS: The European Molecular Biology Open Software Suite, 2000, Trends in Genetics 16, (6) pp 276-277, versions 6.3.1 available from EMBnet at embnet.org/resource/emboss and emboss.sourceforge.net, among other sources) using default gap penalties and scoring matrices (EBLOSUM62 for protein and EDNAFULL for DNA). Equivalent programs may also be used. By “equivalent program” is intended any sequence comparison program that, for any two sequences in question, generates an alignment having identical nucleotide residue matches and an identical percent sequence identity when compared to the corresponding alignment generated by needle from EMBOSS version 6.3.1.

Additional mathematical algorithms are known in the art and can be utilized for the comparison of two sequences. See, for example, the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLAST programs of Altschul et al. (1990) J. Mol. Biol. 215:403. BLAST nucleotide searches can be performed with the BLASTN program (nucleotide query searched against nucleotide sequences) to obtain nucleotide sequences homologous to nucleic acid molecules of the invention, or with the BLASTX program (translated nucleotide query searched against protein sequences) to obtain protein sequences homologous to nucleic acid molecules of the invention. BLAST protein searches can be performed with the BLASTP program (protein query searched against protein sequences) to obtain amino acid sequences homologous to protein molecules of the invention, or with the TBLASTN program (protein query searched against translated nucleotide sequences) to obtain nucleotide sequences homologous to protein molecules of the invention. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) (“A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Hemkoff et al. (1992) Proc. Natl. Acad. Sci. USA 89: 10915-10919). The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) (Nucleic Acids Res. 25:3389-3402) and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) (Nucleic Acids Res. 25:3389-3402).

As indicated, mutant polypeptides disclosed herein are either non-functional or have reduced function as relative to the corresponding wild type polypeptides. The reduction in function can comprise any statistically significant reduction, for example a reduction of about 20%, 25%, 30%, 35%, 40%, 45%, 50%, 55%, 60%, 65%, 70% 75%, 85%, 90%, 95% in function relative to a control. Methods of determining the function of the polypeptides are known and further described below.

An “endogenous” or “native” gene or protein sequence refers to a non-recombinant sequence of an organism as the sequence occurs in the organism before human-induced mutation of the sequence. A “mutated” or “mutant” sequence refers to a human-altered sequence. Examples of human-induced mutation include exposure of an organism to a high dose of chemical, radiological, or insertional mutagen for the purposes of selecting mutants, as well as recombinant alteration of a sequence. Examples of human-induced recombinant alterations can include, e.g., fusions, insertions, deletions, and/or changes to the sequence.

The term “promoter” refers to regions or sequence located upstream and/or downstream from the start of transcription and which are involved in recognition and binding of RNA polymerase and other proteins to initiate transcription. A “plant promoter” is a promoter capable of initiating transcription in plant cells. A plant promoter can be, but does not have to be, a nucleic acid sequence originally isolated from a plant.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

A polynucleotide or polypeptide sequence is “heterologous to” an organism or a second sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety).

“Recombinant” refers to a human manipulated polynucleotide or a copy or complement of a human manipulated polynucleotide. For instance, a recombinant expression cassette comprising a promoter operably linked to a second polynucleotide may include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology, Volumes 1-3, John Wiley & Sons, Inc. (1994-1998)). In another example, a recombinant expression cassette may comprise polynucleotides combined in such a way that the polynucleotides are extremely unlikely to be found in nature. For instance, human manipulated restriction sites or plasmid vector sequences may flank or separate the promoter from the second polynucleotide. Polynucleotides can be manipulated in many ways and are not limited to the examples above.

A “transgene” is used as the term is understood in the art and refers to a heterologous nucleic acid introduced into a cell by human molecular manipulation of the cell's genome (e.g., by molecular transformation). Thus a “transgenic plant” is a plant comprising a transgene, i.e., is a genetically modified plant. The transgenic plant can be the initial plant into which the transgene was introduced as well as progeny thereof whose genome contain the transgene.

An “expression cassette,” used interchangeably with “expression vector,” refers to a nucleic acid construct, generated recombinantly or synthetically, with a series of specified nucleic acid elements that permit transcription of a particular nucleic acid in a host cell. The expression cassette can be part of a plasmid, virus, or nucleic acid fragment. Typically, the expression vector includes a nucleic acid to be transcribed operably linked to a promoter.

The term “plant” includes whole plants, shoot vegetative organs/structures (e.g. leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A “subject plant or plant cell” is a plant or plant cell in which a genetic modification has been made to a polynucleotide of interest or is a plant or plant cell that is descended from a plant or cell so altered and which comprises the alteration. A “control” or “control plant” or “control plant cell” is a plant or plant cell that provides a reference for measuring changes in phenotype of the subject plant or plant cell. A control plant or plant cell can be, for example: (a) a wild-type plant or plant cell, i.e., of the same genotype as the starting material for the genetic alteration which resulted in the subject plant or cell; (b) a plant or plant cell of the same genotype as the wild-type plant or plant cell but which has been transformed with a null construct (i.e., with a construct which has no known effect on the trait of interest, such as a construct comprising a marker gene); (c) a plant or plant cell that is a non-transformed segregant among progeny of a subject plant or plant cell; (d) a plant or plant cell genetically identical to the subject plant or plant cell but that is not/has not been exposed to conditions or stimuli that would induce expression of the gene of interest; or (e) the subject plant or plant cell itself, under conditions in which the gene of interest is not expressed.

An “elite” plant is any plant from an elite line, such that an elite plant is a representative plant from an elite variety. In some embodiments, the soybean plant comprising a polynucleotide encoding any one of the polypeptides disclosed herein is an elite soybean plant. Non-limiting examples of elite soybean varieties that are commercially available to farmers or soybean breeders include: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPR0144RR, BPR 4077NRR and BPR 4390NRR (Bio Plant Research, Camp Point, Ill., USA); DKB 17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minnesota, USA); 90M01, 91M30, 92M33, 93M11, 94M30, 95M30, 97B52, P008T22R2; P16T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRR/STS (Soygenetics, LLC, Lafayette, Ind., USA); S00-K5, S11-L2, S28-Y2, S43-B1, S53-A1, S76-L9, S78-G6, S0009-M2; S007-Y4; S04-D3; S14-A6; S20-T6; S21-M7; S26-P3; S28-N6; S30-V6; S35-C3; S36-Y6; S39-C4; S47-K5; S48-D9, S52-Y2; S58-Z4; S67-R6; S73-S8; and S78-G6 (Syngenta Seeds, Henderson, Ky., USA); Richer (Northstar Seed Ltd. Alberta, CA); 14RD62 (Stine Seed Co. Ia., USA); or Armor 4744 (Armor Seed, LLC, Ar., USA).

As used herein, the term “allele” refers to a variant or an alternative nucleotide sequence of a gene or at a particular genetic locus. Such an allele can be considered (i) wild-type or (ii) mutant if one or more mutations or edits are present in the nucleic acid sequence of the mutant allele relative to the wild-type allele. In diploids, a single allele is inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

A mutant allele for a gene may have a reduced or eliminated activity or expression level for the gene relative to the wild-type allele. For diploid organisms such as corn and soy, a first allele can occur on one chromosome, and a second allele can occur at the same locus on a second homologous chromosome. If one allele at a locus on one chromosome of a plant is a mutant allele and the other corresponding allele on the homologous chromosome of the plant is wild-type, then the plant is described as being heterozygous for the mutant allele. However, if both alleles at a locus are mutant alleles, then the plant is described as being homozygous for the mutant alleles. A plant homozygous for mutant alleles at a locus may comprise the same mutant allele or different mutant alleles if heteroallelic or biallelic.

“Allelic variation” refers to the phenomenon of variation in the sequence form of an allele at a given genetic locus. Allelic variation results in the creation of two or more allelic variants. The variants may be naturally occurring and reflective of genetic differences among individuals of the same species. Such natural variations can occur as a result of natural breeding patterns. Alternatively, the variants may be non-naturally occurring, and artificially created (e.g., by a breeder or a scientist), such as using mutagenesis and/or gene editing techniques. In embodiments of the invention, allelic variants of the soybean gene (e.g., any one of GmCOL2a, GmCOL2b, GmFT4, GmFT5a, and/or GmFT5b) are created through gene editing methods that result in the introduction of a mutation. In additional or alternative embodiments of the invention, allelic variants of the soybean GmCOL2a, GmCOL2b, GmFT4, GmFT5a, and/or GmFT5b genes may be created through chemical mutagenesis, transposon insertion or excision, or any other known mutagenesis technique.

In exemplary embodiments, the mutation introduced into the GmCOL2a, GmCOL2b, GmFT4, GmFT5a, and/or GmFT5b locus/loci is an allele replacement, one or a plurality of base pair insertions, or one or a plurality of base pair deletions. The base pair insertions or base pair deletions may include a 3n base mutation wherein a multiple of 3 base pairs are deleted or inserted (e.g., insertion or deletion of 3 bp, 6 bp, 9 bp, 12 bp, 15 bp, 18 bp, etc.), thereby not affecting the reading frame of the gene. Alternatively, the base pair insertion or deletion may not be a multiple of 3 base pairs (e.g., an insertion or deletion of 2 bp, 4 bp, 5 bp, 7 bp, 11 bp, etc.), thereby affecting the reading frame of the gene.

In particular embodiments, the mutation is a truncation mutation wherein the mutation can result in the introduction of a stop codon into the gene at a location earlier than intended. Transcription of the resulting mutant allele is terminated at the earlier than intended stop codon, resulting in a truncated protein that is shorter than the corresponding wild-type protein.

As used herein, an “allelic combination” refers to the specific combination of alleles present at more than one characterized location or loci. Exemplary embodiments of the invention include a plurality of allelic combinations at the GmCOL2a and GmCOL2b loci or at the GmFT5a and GmFT5b loci.

In embodiments of the invention, the allelic combination of a plant at the combination of loci, for example, at loci of GmCOL2a and GmCOL2b may be determined via molecular marker-based assays, such as a first assay of the DNA of the plant indicative of a type of mutation introduced at the GmCOL2a locus and a second assay of the DNA indicative of a type of mutation introduced at the GmCOL2b locus. In embodiments, the allelic combination is indicative of a change in flowering time of the plant relative to a control plant not comprising the allelic combination (e.g., a control plant comprising one or more of the wild-type alleles or comprising the ninth allelic combination of wild type alleles at both loci).

A “dominant maturity allele” is an allele that, when present either in single copy (heterozygous) or two copies (homozygous), affects the maturity of the plant. A “recessive maturity allele” is an allele that affects the maturity of the plant only when present in two copies (homozygous), and does not affect the maturity of a plant when present in a single copy (heterozygous).

As used herein, a modified plant that flowers or matures “slightly earlier” (or that has “slightedly accelerated” flowering or maturity, or that has slightly reduced flowering time and/or maturity) than a control plant has a flowering time and/or maturity time that is between 1 and 10 days shorter than the control plant (e.g., shorter than that of the control plant by at least 1 day, 2 days, 3 days, 4 days, 5 days, 6 days, 7 days, 8 days, 9 days or 10 days). In embodiments, the flowering time of a modified plant is slightly earlier than the control plant if the flowering time is shorter by 1-2 days, 1-3 days, 1-4 days, 1-5 days, 1-6 days, 1-7 days, 1-8 days, 1-9 days or 1-10 days.

As used herein, a modified plant that flowers or matures “slightly later” (or that has “slightedly delayed” flowering and/or maturity, or that has slightly increased flowering time and/or maturity) than a control plant has a flowering time and/or maturity time that is between 1 and 10 days longer than the control plant (e.g., shorter than that of the control plant by at least 1 day, 2 days, 3 days, 4 days, 5 days, 6 days, 7 days, 8 days, 9 days or 10 days). In embodiments, the flowering time of a modified plant is slightly later than the control plant if the flowering time is shorter by 1-2 days, 1-3 days, 1-4 days, 1-5 days, 1-6 days, 1-7 days, 1-8 days, 1-9 days or 1-10 days.

As used herein, a modified plant that flowers and/or matures “significantly earlier” (or has “significantly accelerated” flowering or maturity, or has significantly reduced flowering time and/or maturity time) than the control plant has a flowering time and/or maturity time that is at least 10 days shorter than that of the control plant, such as between 10-100 days shorter than the control plant (e.g., shorter than that of the control plant by at least 10 days, 10-20 days 10-30 days, 10-40 days, 10-50 days, 10-60 days, 10-70 days, 10-80 days, 10-90 days or 10-100 days or any range therebetween such as 20-30 days, 20-40 days, 30-40 days, 40-50 days, 50-60 days, 70-80 days, 80-90 days, 90-100 days, and so on).

In comparison, a modified plant that flowers or matures “significantly later” or (has “significantly delayed” flowering and/or maturity, or has significantly increased flowering time and/or maturity time) than the control plant has a flowering time and/or maturity time that is at least 10 days longer than that of the control plant, such as between 10-100 days longer than the control plant (e.g., longer than that of the control plant by at least 10 days, 10-20 days 10-30 days, 10-40 days, 10-50 days, 10-60 days, 10-70 days, 10-80 days, 10-90 days or 10-100 days or any range therebetween such as 20-30 days, 20-40 days, 30-40 days, 40-50 days, 50-60 days, 70-80 days, 80-90 days, 90-100 days, and so on).

As used herein, the term “photoperiodic response” or “photoperiodism” refers to the physiological reaction of a plant to the relative lengths of light and dark periods. Photoperiod responsive plants may be “short-day”, “long-day” or “day-neutral” plants. Photoperiodism affects flowering by inducing the shoot to produce floral buds instead of leaves and lateral buds. Soybean, for example, is a short-day (SD) plant. In embodiments of the invention, soybean flowering time is assessed. Short day plants flower when the night lengths exceed their critical photoperiod and cannot flower under short nights. They require a continuous period of darkness before floral development can begin. Natural nighttime light, such as moonlight or lightning, is not of sufficient brightness or duration to interrupt flowering. Typically, short-day (i.e. long-night) plants flower as days grow shorter (e.g., late summer and fall in the northern hemisphere). The length of the dark period required to induce flowering differs among species and varieties of a species. Long-day plants flower when the night length falls below their critical photoperiod. These plants typically flower as days get longer (e.g., late spring and early summer in the northern hemisphere).

As used herein, “flowering time” or “days to flowering” is an estimate of a duration (e.g., in terms of hours, days, weeks, etc.) elapsed between initiation of first flowering and seed emergence. In embodiments of the invention, flowering time of a soybean plant is modified or altered, relative to a control plant, through the introduction of novel non-naturally occurring alleles in genes involved in soybean maturity, particularly GmCOL2a, GmCOL2b, GnFT4, GmFT5a, and/or GmFT5b genes. In particular embodiments, flowering time is defined as a number of days elapsed for a soybean plant to transition from a VE stage (e.g., seeds emergence wherein cotyledons have been pulled through the soil surface for at least 50% of the seeds) to an R1 stage (e.g., beginning of flowering wherein at least 50% of the plants have at least one flower on any node).

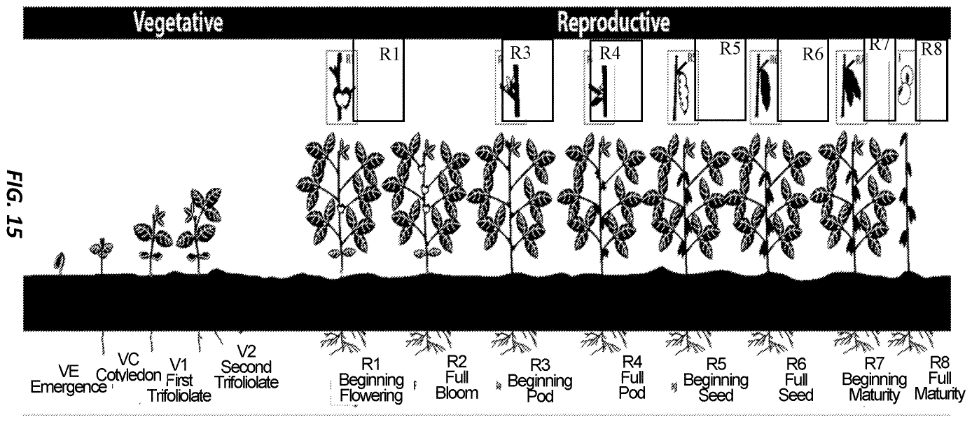

As used herein, maturity time or post flowering time is defined as the number of days elapsed for a soybean plant to transition from the R1 stage (e.g., beginning of bloom wherein there is one open flower at any node on the main stem) to an R7 stage (wherein any pod has reached a mature pod color) or from the R1 stage to an R8 stage (wherein 95% of the pods have reached their mature pod color). A description of the various development stages of a soybean plant and mature soy pod coloration is provided at FIG. 15 as reference.

### II. Introduction

It may be desirable to alter the flowering time and/or maturity time of photoperiod-responsive and agronomically important plants such as soybean to enable a wider geographic range of cultivation. In some examples, it may be desirable to accelerate or advance or shorten flowering time and/or maturity time in soybean so that seeds can be produced and harvested sooner, and/or produced and harvested in higher latitudes including regions with longer days. In other examples, it may be desirable to delay or lengthen flowering time and/or maturity time in soybean so that seeds can be produced and harvested in lower altitudes, including regions with shorter days. This disclosure provides useful compositions and methods that can be used to alter flowering and/or maturity time in soybean.

In some embodiments, provided herein are maturity genes such as GmCOL2a, GmCOL2b, GnFT4, GmFT5a, and/or GmFT5b, which can confer phenotypic traits including one or more or a combination of flowering time, post-flowering time, relative maturity, maturity time, maturity group and number of days from flowering of the soybean plant to beginning of maturity. In particular embodiments, the phenotypic trait measured is a flowering time and includes a measure of time elapsed between the VE and R1 phase of a modified soybean plant (see FIG. 15 for the various stages) relative to a control plant. In another embodiment, the phenotypic trait measured is a maturity time and includes a measure of time elapsed between the R1 and R7 phase, or R1 and R8 phase, of the modified soybean plant (see FIG. 15 for the various stages) relative to a control plant. The number of days may vary, relative to a control plant comprising wild-type alleles at both loci, based on the specific allelic combination of the plant.

In some embodiments, the method and compositions disclosed herein include making a genomic modification to one or more or a combination of the following genes: GmCOL2a, GmCOL2b, GmFT4, GmFT5a, and/or GmFT5b genes. The genomic sequence, cDNA sequence, and protein sequence corresponding to each of the above gene are listed in Table 1.

TABLE 1
Sequences

| | Genomic | CDNA | Protein |
|---|---|---|---|
| GmCOL2a | SEQ ID NO: 15 | SEQ ID NO: 16 | SEQ ID NO: 17 |
| GmCOL2b | SEQ ID NO: 21 | SEQ ID NO: 22 | SEQ ID NO: 23 |
| GmFT4 | SEQ ID NO: 27 | SEQ ID NO: 56 | SEQ ID NO: 28 |
| GmFT5a | SEQ ID NO: 52 | SEQ ID NO: 39 | SEQ ID NO: 40 |
| GmFT5b | SEQ ID NO: 54 | SEQ ID NO: 35 | SEQ ID NO: 36 |

Provided herein are methods to alter flowering and/or maturity time by modifying the genome of the plant. In some embodiments, the plant is knocked out of one or more of the GmCOL2a, GmCOL2b, GmFT4, GmFT5a, and/or GmFT5b genes. In some embodiments, the plant is edited to express one or more mutant polypeptides expressed from these genes that have reduced function or reduced expression as compared to their corresponding wild type polypeptides. In these embodiments, the plant does not express the corresponding wild type polypeptide(s).

Also provided herein are methods to alter flowering and/or maturity time by reducing or suppressing the expression or activity one or more of the GmCOL2a, GmCOL2b, GnFT4, GmFT5a, and/or GmFT5b genes. A number of methods can be used to suppress or silence gene expression of the above referenced genes in the soybean plant. In some embodiments, reduction or suppression of gene expression is achieved by introducing an expression cassette encoding RNAi (e.g., siRNA, miRNA) comprising a polynucleotide sequence at least substantially identical to the target gene linked to the complementary polynucleotide sequence. The transcribed RNAi molecule hybridizes to the target gene and silence its expression. Other gene silencing methods can also be used, such as, microRNA (miRNA), anti-sense, cosuppression, viral-suppression, hairpin suppression, stem-loop suppression, and the like.

In some embodiments, a mutant polypeptide expressed by a plant comprising the genomic modification shares less than 20%, less than 15%, or less than 10% identity with the corresponding wild-type polypeptide. For example, a mutant GmCOL2a polypeptide shares less than 20% identity with corresponding wild type GmCOL2a polypeptide (SEQ ID NO: 17); a mutant GmCOL2b polypeptide shares less than 20% identity with corresponding wild type GmCOL2b polypeptide (SEQ ID NO: 23); a mutant GmFT4 polypeptide shares less than 20% identity with corresponding wild type GmFT4 polypeptide (SEQ ID NO: 28); a mutant GmFT5a polypeptide shares less than 20% identity with corresponding wild type GmFT5a polypeptide (SEQ ID NO: 40); and a mutant GmFT5b polypeptide shares less than 20% identity with corresponding wild type GmFT5b polypeptide (SEQ ID NO: 36).

In some embodiments, a mutant GmCOL2a polypeptide shares at least 70%, at least 80%, at least 90%, at least 95% amino acid sequence identity with the mutant GmCOL2a polypeptide (SEQ ID NO: 20). In some embodiments, a mutant GmCOL2b polypeptide has at least 70%, at least 80%, at least 90%, at least 95% amino acid sequence identity with the mutant GmCOL2b polypeptide (SEQ ID NO: 26). In some embodiments, a mutant GmFT4 polypeptide has at least 70%, at least 80%, at least 90%, at least 95% amino acid sequence identity with the mutant GmFT4 polypeptide (SEQ ID NO: 30 or 32). In some embodiments, a mutant GmFT5a polypeptide has at least 70%, at least 80%, at least 90%, at least 95% amino acid sequence identity with the mutant GmFT5a polypeptide (SEQ ID NO: 42). In some embodiments, a mutant GmFT5b polypeptide has at least 70%, at least 80%, at least 90%, at least 95% amino acid sequence identity with the mutant GmFT5b polypeptide (SEQ ID NO: 38).

The genome modification approach as discussed herein can also be utilized to generate a combination of mutant alleles in a single plant. For example, the genome of soybean plant can be modified to comprise any, one, two, or three of (i) a mutant GmCOL2a allele, (ii) a mutant GmCOL2b allele, (iii) a mutant GmFT4 allele, (iv) a mutant GmFT5a allele, and (v) a mutant GmFT5b allele. In some embodiments the modified plants express one, two, or three of the mutant proteins: (i) a mutant GmCOL2a allele, (ii) a mutant GmCOL2b allele, (iii) a mutant GmFT4 allele, (iv) a mutant GmFT5a allele, and (v) a mutantGmFT5b allele.

In some embodiments, the soybean plant is a double mutant comprising, for example, a soybean plant in which the genome has been modified to comprise both a mutant GmCOL2a allele and a mutant GmCOL2b allele. In some exemplary embodiments, the mutant GmCOL2a allele comprises a 398-bp deletion and encodes a mutant polypeptide with an amino acid sequence set forth in SEQ ID NO: 20, and the mutant GmCOL2b allele comprises a 1-bp deletion and encodes a mutant polypeptide with an amino acid sequence set forth in SEQ ID NO: 26. In some embodiments, the double mutant soybean plant has a genome that has been modified to comprise both a mutant Gmft5a allele and a mutant Gmft5b allele. In some exemplary embodiments, the mutant Gmft5a allele comprises a 1-bp insertion and encodes a mutant polypeptide with an amino acid sequence set forth in SEQ ID NO: 42, and the mutant Gmft5b allele comprises an 8-bp deletion and encodes a mutant polypeptide with an amino acid sequence set forth in SEQ ID NO: 38.

In some embodiments, acceleration of flowering and/or maturity time is performed by decreasing the expression (e.g., knocking out or knocking down) one or more of GmCOL2a, GmCOL2b, and GnFT4. In some embodiments delay flowering and/or maturity time is performed by decreasing the expression (e.g., knocking out or knocking down) one or more of GmnFT5a and/or GmnFT5b.

Various methods of editing the genes in a plant are also provided. In some embodiments, the wild type allele of one or more of the GmCOL2a, GmCOL2b, GmFT4, GmFT5a, and/or GnFT5b genes are deleted from the genome. In some embodiments, the wild type genes have been modified to result in mutant alleles that encode non-functional polypeptides or polypeptide having reduced function. Exemplary mutant alleles of the genes are also provided at SEQ ID NOS:18, 24, 29, 31, 55, and 53. A polypeptide is deemed to have reduced function if compared to the corresponding wild type polypeptide, its activity is reduced to less than 70%, less than 60%, less than 50%, less than 40%, less than 30%, less than 20%, or less than 10% of the activity of the wild type protein when assayed under the same assay conditions.

In some embodiments, the wild type GmCOL2a, GmCOL2b, GmFT4, GmFT5a, and/or GnFT5b genes have been modified to result in mutant alleles that encode non-functional polypeptides or polypeptide having reduced expression. Exemplary mutant alleles of the genes are also provided in SEQ ID NOS: 18, 24, 29, 31, 55, and 53. A polypeptide is deemed to have reduced expression if compared to the corresponding wildtype polypeptide, its expression is reduced to less than 70%, less than 60%, less than 50%, less than 40%, less than 30%, less than 20%, or less than 10% of the expression of the wild type protein when assayed under the same assay conditions. A gene is deemed to have reduced expression if compared to the corresponding wild type gene or allele, the transcript level, or transcribed protein level, is reduced to less than 70%, less than 60%, less than 50%, less than 40%, less than 30%, less than 20%, or less than 10%.

In some embodiments, genomic modification is a performed on a plant having a genomic DNA sequence with at least 85%, 90%, 91%, 92%, 93%, 94%, 95%, 96%, 97%, 98%, or 99% identity to at least one of SEQ ID NOS: 15, 21, 27, 52, and/or 54. In some embodiments, genomic modification is a performed on a plant having a genomic DNA sequence with at least 85%, 90%, 91%, 92%, 93%, 94%, 95%, 96%, 97%, 98%, or 99% identity to at least one of SEQ ID NOS: 16, 22, 56, 35, and/or 39. In some embodiments, the genomic DNA sequence comprises a nucleic acid sequence as set forth in any one of SEQ ID NOS: 1, 6, 43, 49, and/or 46.

In some embodiments, the genomic modification results in the plant having decreased expression and/or activity of a polypeptide comprising: (a) an amino acid sequence comprising at least 85%, 90%, 91%, 92%, 93%, 94, 95%, 96%, 97%, 98%, or 99% identity to at least one of SEQ ID NOS: 17, 23, 28, 36, and/or 40, or (b) an amino acid sequence set forth in at least one of SEQ ID NOS: 17, 23, 28, 36, and/or 40. In some embodiments, the decrease in expression as compared to the control plant is at least 80%, at least 90%, at least 95%.

In some embodiments, the mutant polypeptide expressed by a plant comprising the genomic modification lacks one or more conserved domains of the wildtype polypeptides or comprise an inactivating mutation therein (i.e., a mutation that substantially or entirely eliminates the function of the domain). Conserved domains of the GmCOL2a, GmCOL2b, GmFT4, GmFT5a, and/or GmFT5b polypides are known and also described in Section III of this disclosure. In some embodiments, gene editing approaches that target sequences in the conserved domains of one or more of GmCOL2a (SEQ ID NO: 17), GmCOL2b (SEQ ID NO: 23), GmFT4 (SEQ ID NO: 28), GmFT5a (SEQ ID NO: 40), and/or GmFT5b (SEQ ID NO: 36) polypides can be used to produce these mutant polypeptides. See, Section V, subsection I. One of ordinary skill in the art would be able to modulate plant flowering and development by gene editing one or more of the target sequences in the conserved domains of one or more of GmCOL2a (SEQ ID NO: 17), GmCOL2b (SEQ ID NO: 23), GmFT4 (SEQ ID NO: 28), GmFT5a (SEQ ID NO: 40), and/or GmFT5b (SEQ ID NO: 36) polypeptides.

### Stages and Long Day (LD) and Short Day (SD) Conditions

There are two distinct growth phases in soybean development: the vegetative (V) stages, which include from emergence through flowering, and reproductive (R) stages, which include from flowering through maturity. These stages are determined by classifying leaf, flower, pod, and/or seed development. A brief description of various plants stages is shown provided in Table 2 and as shown at FIG. 15. The information is also available at extension.umn.edu/growing-soybean/soybean-growth-stages #reproductive-phase-%28table-2%29-539861, the content of which is a very incorporated by reference.

TABLE 2
Plant stages

| Stage | Description |
|---|---|
| VE | Emergence: Cotyledons above the soil surface. |
| VC | Cotyledon: Unifoliolate leaves sufficiently unroll, so the leaf edges |
| | do not touch. |
| V1 | First node: Fully developed leaves at unifoliolate node. |
| V(n) | nth-node: The “n” represents the number of nodes on the main |
| | stem with fully developed leaves, beginning with the unifoliolate |
| | leaves. |
| R1 | Beginning bloom: One open flower at any node on the main stem. |
| R2 | Full bloom: Open flower at one of the two uppermost nodes on the |
| | main stem with a fully developed flower. |
| R3 | Beginning pod: Three-sixteenths-inch-long pod at one of the four |
| | uppermost nodes on the main stem with a fully developed leaf. |
| R4 | Full pod: Three-quarter-inch-long pod at one of the four uppermost |
| | nodes on the main stem with a fully developed leaf. |
| R5 | Beginning seed: One-eighth-inch-long seed in a pod at one of the |
| | four uppermost nodes on the main stem with a fully developed leaf. |
| R6 | Full seed: Pod containing a green seed that fills the pod cavity at |
| | one of the four uppermost nodes on the main stem with a fully |
| | developed leaf. |
| R7 | Beginning maturity: One normal pod on the main stem that has |
| | reached its mature pod color. |
| R8 | Full maturity: 95 percent of the pods have reached their mature |
| | pod color. After R8, five to 10 days of drying weather are required |
| | to reduce soybean moisture levels to less than 15 percent. |

In many plant species, the timing of flowering is correlated with photoperiod (aka., the length of daylight). Some plants prefer long daylight (LD) conditions, i.e., more than 12 hours of daylight or less than 12 hours of uninterrupted darkness each day, to flower. These plants are referred to as long day (LD) plants. Exemplary LD plants include, but are not limited to, carrots, lettuce, potato, spinach, and turnips. Some plants prefer short daylight (SD) conditions, i.e., less than 12 hours of daylight or more than 12 hours of uninterrupted darkness each day, to flower. These plants are referred to as short day (SD) plants. Exemplary SD plants include, but are not limited to, soybean. Notably, although soybean is known as a short-day plant species, soybean plants can still flower under long-day (LD) conditions, albeit much later than in SD conditions (Cai et al., Plant Biotechnology J. 2020 January; 18(1): 298-309). Yet other plants do not initiate flowering based on length of daylight; these plants are referred to as day neutral (DN) plants. Exemplary DN plants include, but are not limited to, cabbage, corn, cucumber, and kale. In exemplary embodiments, a plant grown under LD conditions (16 h light/8 h dark in a 24-hour period). In exemplary embodiments, a plant grown under SD conditions (12 h light/12 h dark in a 24-hour period). In the context of this disclosure, it is understood that reference to a “day” includes any 24-hour period.

### Altered Flowering Time

The methods and compositions discussed herein can be used to alter soybean flowering time. The flowering time of a soybean plant for the purpose of this application reflects how soon a soybean plant initiates flowering. The flowering time is typically determined by counting the number of days between the VE stage and the R1 stage (FIG. 15). The number of days from the VE stage to a particular stage in plant development is referred to as Date After Emergence (DAE). The flowering time for wild type soybean is typically 38 to 42 DAE under LD conditions and 20 to 23 DAE under SD conditions. As used herein, the term “altered flowering” refers to the flowering time (DAE) has been increased or decreased as compared to a control plant. A soybean plant flowers later than a control plant if its flowering time is longer than a control plant. Conversely, a soybean plant flowers earlier than a control plant if its flowering time is shorter than a control plant.

### Altered Maturity Time

The maturity of the soybean plant is indicated by the formation of pods. The maturity time reflects how soon a plant forms mature pod on the main stem. Unless stated otherwise for the purpose of this application, the maturity time of soybean plant is measured by the number of days between the VE stage and to the R7 stage (the time at which the first pod has reaches the maturity color on the main stem). The maturity time for a wild type soybean plant is typically 136 to 142 DAE under LD conditions and 70 to 73 DAE under SD conditions. A soybean plant matures later than a control plant if its maturity time is longer than a control plant. Conversely, a soybean plant matures earlier than a control plant if its maturity time is shorter than a control plant. As used herein, the term “altered maturity time” refers to the maturity time (DAE) has been increased or decreased as compared to a control plant.

### III. Polynucleotides and Polypeptides that Confer Accelerated Flowering

### A. GmCOL2a and GmCOL2b

GmCOL2a/GmCOL2b are soy orthologs that are in the same family as the Arabidopsis CONSTANS (CO) protein. CO plays a central role in photoperiodic flowering control of Arabidopsis. GmCOL2a and GmCOL2b can complement the late flowering effect of the CO mutant in Arabidopsis. (Wu, F. et al., PLoS One, 9(1): e85754, Jan. 21, 2014, doi.org/10.1371/journal.pone.0085754). GmCOL2a and GmCOL2b show a diurnal expression rhythm under SD conditions, but their rhythmic expression patterns are less clear under LD conditions. For example, expression of GmCOL2a and GmCOL2b under SD conditions peak after dusk (T4: 18:30) and decline during the night; but appeared to peak at two time points: T4 (18:30) and T6 (2:30) under LD conditions. It has also been reported that cool temperatures may upregulate GmCOL2b expression, particularly at the fourth trifoliate-leaf stage in soybean. (Zhang, J. et al., Front. Plant Sci., Vol. 11, Art. 429, 15 Apr. 2020, doi.org/10.3389/fpls.2020.00429). GmCOL2a and GmCOL2b share 83.78% amino acid similarity in the coding region. The wild type GmCOL2a has a genomic sequence of SEQ ID NO: 15 and a coding sequence of SEQ ID NO: 16. It encodes a protein with the amino acid sequence of SEQ ID NO: 17. The wild type GmCOL2b has a genomic sequence of SEQ ID NO: 21 and a coding sequence of SEQ ID NO: 22, which encodes a polypeptide with a sequence of SEQ ID NO: 23.

The GmCOL2a gene or an allele thereof, is in reference to GLYMA_08G255200 (soybase.org). GmCOL2a gene is located on the chromosome 8 and encodes a polypeptide that is homologous to Arabidopsis CONSTANS (CO) protein. The GmCOL2 polypeptide comprises a B-box zinc finger domain and a CCT motif and suppresses photoperiodic flowering in soybean under long day condition (Cao et al., Plant Cell Physiol. 56 (12), 2409-2422 (2015)).

The GmCOL2b gene or an allele thereof, is in reference to GLYMA_18G278100 (soybase.org) and is involved in soybean flowering transition. GmCOL2b is also referred to as CONSTANCE-like 2b and is located on the chromosome 18. It encodes a polypeptide that is homologous to Arabidopsis CONSTANS (CO) protein. The GmCOL2b polypeptide comprises a B-box zinc finger domain and a CCT motif and belongs to the GATA-4/5/6 transcription factor family (Zhang et al. Front Plant Sci. 2020, April 15:11:429, doi: 10.3389/fpls.2020.00429).

The inventors of this disclosure have found surprisingly that knocking out GmCOL2a and GmCOL2b individually or together can significantly accelerate flowering and/or maturity time in soybean plants (that is, reduce the flowering time relative to the control plant). The effect of acceleration on flowering and/or maturity time is even more significant when both genes were knocked out in the same plant. In some cases, the soybean plant in which GmCOL2a and/or GmCOL2b are knocked out flower and/or mature 2-40 days, e.g., 3-30 days, 4-25 days, 5-20 days, or 5-17 days, earlier than a control plant. In some cases, a soybean plant in which GmCOL2a and/or GmCOL2b are knocked out flower 2 days, 3 days, 4 days, 5 days, 6 days, 7 days, 8 days, 9 days, 10 days, or more, earlier than the control plant. In some cases, a soybean plant in which both GmCOL2a and GmCOL2b are knocked out flower significantly earlier than a control plant, for example, 10 days, 11 days, 12 days, 13 days, 14 days, 15 days, 16 days, 17 days, 18 days, 19 days, 20 days or more, earlier than a control plant.

For purpose of this disclosure, knocking out a gene refers to a plant or plant cell in which the wild type gene is completely removed/deleted or mutated to form a mutant allele encoding a non-functional protein (null mutant). In some embodiments, the plant or plant cell is edited to express a protein having reduced function relative to the corresponding wild-type protein. Methods and compositions disclosed herein can be used to decrease expression and/or decrease activity of (e.g., knockdown or knockout) one or more of the polypeptides disclosed herein, for example, GmCOL2a, GmCOL2b, GmFT4, GmFT5a, and/or GmFT5b, as further described in Section V below.

Methods of introducing genomic modification to plants are known, and exemplary approaches are also described in Section V of this application, entitled “Methods for producing a plant variety that has altered flowering and/or maturity time.” In some embodiments, the genomic modification is performed by CRISPR/Cas9-mediated targeted mutagenesis, using sgRNAs that target a sequence in GmCOL2a and/or GmCOL2b. In one exemplary approach, one or more vectors encoding Cas9 and sgRNA containing the target-binding sequence are introduced into a soybean plant. The plants comprising expressing Cas9 and sgRNA can be selected based on the selection marker in the vectors and verified by PCR or sequencing. The resulting mutant GmCOL2a or GmCOL2b allele can be determined by sequencing. In one illustrative example, editing of GmCOL2a uses the reagents in Table 3. In one illustrative example, editing of GmCOL2b uses the reagents in Table 4.

TABLE 3
Reagents for knocking out GmCOL2a

| | |
|---|---|
| GmCOL2a target | TTGGTGGCAGCACCGGCACCTGG |
| sequence | (SEQ ID NO: 1) |
| DNA oligonucleotide | TCGAAGTAGTGATTGTTGGTGGCAGCAC |
| encoding sgRNA-1 | CGGCACCGTTTTAGAGCTAGAA |
| | (SEQ ID NO: 2) |
| DNA oligonucleotide | TTCTAGCTCTAAAACGGTGCCGGTGCTG |
| encoding sgRNA-2 | CCACCAACAATCACTACTTCGA |
| | (SEQ ID NO: 3) |

TABLE 4
Reagents for knocking out GmCOL2b

| | |
|---|---|
| GmCOL2b target | GCAGCAACACTGGCACCACCTGG |
| sequence | (SEQ ID NO: 6) |
| DNA oligonucleotide | TCGAAGTAGTGATTGGCAGCAACACTGGC |
| encoding sgRNA-1 | ACCACCGTTTTAGAGCTAGAA |
| | (SEQ ID NO: 7) |
| DNA oligonucleotide | TTCTAGCTCTAAAACGGTGGTGCCAGTGT |
| encoding sgRNA-2 | TGCTGCCAATCACTACTTCGA |
| | (SEQ ID NO: 8) |

In some embodiments, the mutant allele produced by the gene editing is a non-naturally occurring mutant allele. In some embodiments, the mutant GmCOL2a and/or GmCOL2b allele(s) comprise one or more of the following: a nonsense mutation, an in-frame deletion mutation, a missense mutation, a frameshift mutation, a splice-site mutation, or any combination thereof. In some embodiments, editing of the GmCOL2a and/or GmCOL2b results in the plant with one or more of the following: a protein truncation, a non-functional protein, or a protein with reduced function relative to a protein expressed by the corresponding wild type allele.

In some embodiments, genomic editing of a plant produces a modified plant expressing a mutant GmCOL2a polypeptide comprising: (a) an amino acid sequence that is at least 85%, at least 90%, at least 95%, at least 98%, or at least 99% identical to SEQ ID NO: 20; or (b) an amino acid sequence as set forth in SEQ ID NO: 20. In some embodiments, the modified plant comprises a mutant GmCOL2a allele that is SEQ ID NO: 18 or 19. In some embodiments, the modified plant comprises a mutant GmCOL2a allele having a nucleic acid sequence that is at least 85%, at least 90%, at least 95%, at least 98%, or at least 99% identical to SEQ ID NO: 18 or 19.

In some embodiments, a soybean plant disclosed herein comprising a mutant GmCOL2a allele flowers earlier than a control plant that comprises the wild type GmCOL2a allele. In some embodiments, the GmCOL2a mutant allele comprises a 398-bp deletion and encodes a mutant polypeptide GmCOL2a having an amino acid sequence of SEQ ID NO: 20. In one illustrative embodiment, a plant expressing this mutant polypeptide flowers earlier (e.g., five days) than a control plant that comprises the wild type GmCOL2a under the LD conditions as shown, for instance, in Example 1.

In some embodiments, genomic editing of a plant produces a modified plant expressing a mutant GmCOL2b polypeptide comprising: (a) an amino acid sequence that is at least 85%, at least 90%, at least 95%, at least 98%%, or at least 99% identical to SEQ ID NO: 26; or (b) an amino acid sequence as set forth in SEQ ID NO: 26. In some embodiments, the modified plant comprises a polynucleotide that is SEQ ID NO: 24 or 25. In some embodiments, the modified plant comprises a mutant GmCOL2b allele that is at least 85%, at least 90%, at least 95%, at least 98%%, or at least 99% identical to SEQ ID NO: 24 or 25.

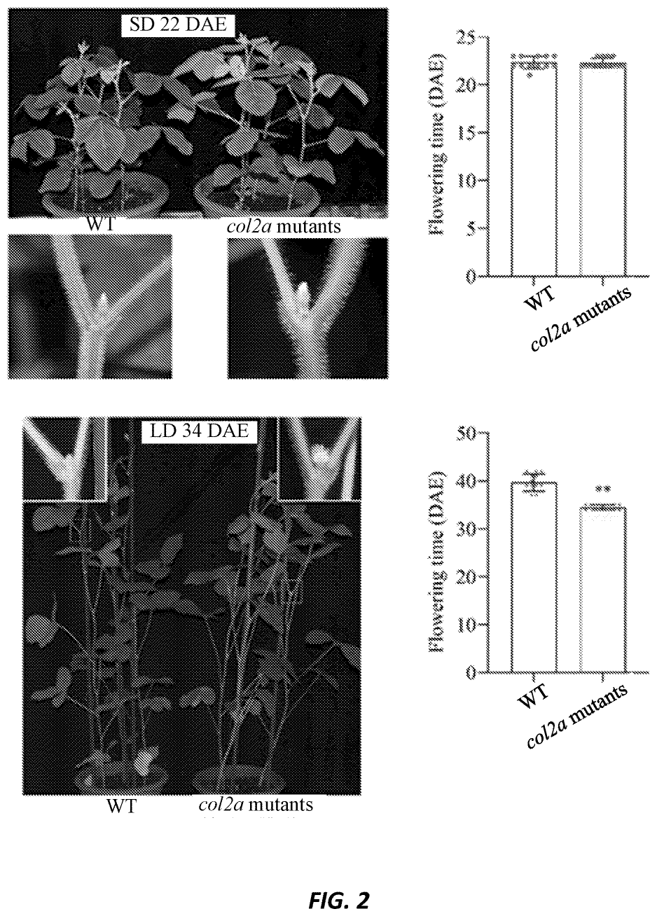

As compared to the genomic sequence of wild type GmCOL2a (SEQ ID NO: 15), the mutant GmCOL2a allele (SEQ ID NO: 18) comprises a 398-bp deletion (at position −318 to 70 bp), using the A of the initiation codon ATG is considered as the position 1. As shown in Example 1 and FIG. 2, the soybean plant expressing this mutant allele flowered 12 days earlier relative to a control plant under LD conditions.

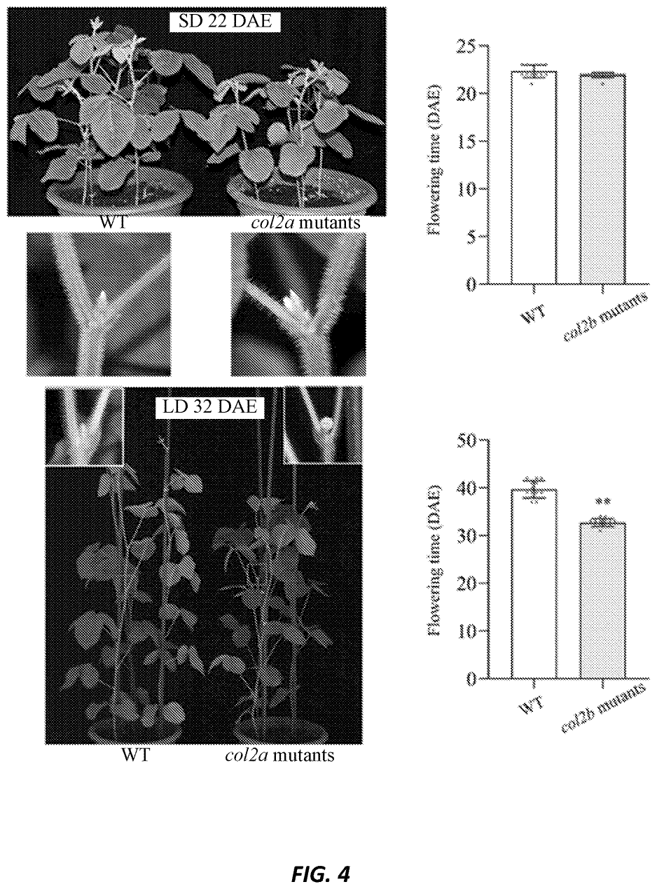

As compared to the genomic sequence of wild type GmCOL2b (SEQ ID NO: 21), the mutant GmCOL2b allele (SEQ ID NO: 24) comprises a 1-bp deletion (at position 48 bp), using the A of the initiation codon ATG is considered as the position 1. As is shown in Example 1 and FIG. 4, the soybean plant expressing this mutant allele flowered 7 days earlier relative to the control plant under LD conditions.

In some embodiments, a soybean plant disclosed herein comprising a mutant GmCOL2b flowers earlier than a control plant that comprises the wild type GnCOL2b. In some embodiments, the GmCOL2b mutant allele comprises a 1-bp deletion and encodes a mutant polypeptide GmCOL2b having an amino acid sequence of SEQ ID NO: 26. In one illustrative embodiment, a plant expressing this mutant protein flowers earlier (e.g., seven days) than a control plant that comprises the wild type GmCOL2b under the LD conditions as shown, for instance, in Example 1.

In some embodiments, the soybean plant is a double mutant comprising, for example, a genome that has been modified to comprise both a mutant GmCOL2a allele and a mutant GmCOL2b allele. In some embodiments, the double mutant soybean plant flowers earlier than a control plant comprising one or both of the wild type GmCOL2a allele and the wild type GmCOL2b allele. In some embodiments, the mutant GmCOL2a allele comprises a 398-bp deletion and encodes an amino acid sequence set forth in SEQ ID NO: 20, and the mutant GmCOL2b allele comprises a 1-bp deletion and encodes a mutant polypeptide having an amino acid sequence set forth in SEQ ID NO: 26. In one illustrative embodiment, the double mutant soybean plant flowered 17 days earlier than a control plant comprising wild type GnCOL2a and GmCOL2b alleles as shown, for instance, in Example 1.

### B. GmFT4

GmFT4 is a homolog of Flowering Locus T. The genomic sequence of GmFT4 is SEQ ID NO: 27 and the coding sequence is SEQ ID NO: 28. The expression of GmFT4 protein (SEQ ID NO: 29) is strongly up-regulated under LD conditions, exhibiting a diurnal rhythm, but down-regulated under SD conditions. Notably, the basal expression level of GmFT4 is elevated when transferred to continuous light, whereas repressed when transferred to continuous dark. GmFT4 is primarily expressed in fully expanded leaves (Zhai et al., PLoS One, 9(2): e89030, Feb. 19, 2014, doi.org/10.1371/journal.pone.0089030).

The inventors of this disclosure have found surprisingly that knocking out GmFT4 can accelerate flowering and/or maturity time in soybean plants. In some cases, the soybean plant in which GmFT4 has been knocked out flower and/or mature 3, 4, 5, 6, 7, or 8 days earlier than a control plant expressing the wild type GmFT4 as shown, for instance, in Example 2.

Methods of introducing genomic modification to plants are known, and exemplary approaches are also described in Section V of this application, entitled “methods for producing a plant variety that has altered flowering and/or maturity time”. In some embodiments, the gene editing of GmFT4 is performed by CRISPR/Cas9-mediated targeted mutagenesis by using sgRNAs that target a sequence in GmCOL2a. In one exemplary approach, one or more vectors encoding Cas9 and sgRNA containing the target-binding sequence are introduced into a soybean plant. The plants comprising expressing Cas9 and sgRNA can be selected based on the selection marker in the vectors and verified by PCR or sequencing. The resulting mutant GmFT4 allele can be determined by sequencing. In one illustrative example, editing of GmFT4 uses the reagents in Table 5.

TABLE 5
Reagents for knocking out GmFT4

| | |
|---|---|
| GmFT4 target sequence | CTTGTTCTTGGACGTATAATAGG |
| | (SEQ ID NO: 43) |
| DNA oligonucleotide | TTGCTTGTTCTTGGACGTATAAT |
| encoding sgRNA-1 | (SEQ ID NO: 44) |
| DNA oligonucleotide | AACATTATACGTCCAAGAACAAG |
| encoding sgRNA-2 | (SEQ ID NO: 45) |

In some embodiments, the mutant allele produced by the gene editing is a non-naturally occurring mutant allele. In some embodiments, the mutant GnFT4 allele comprises one or more of the following: a nonsense mutation, an in-frame deletion mutation, a missense mutation, a frameshift mutation, a splice-site mutation, or any combination thereof. In some embodiments, editing of the genomic sequence of the wild type GnFT4 allele results in the plant with one or more of the following: a protein truncation, a non-functional protein, or a protein with reduced function relative to a protein expressed by the corresponding wild type allele.

In some embodiments, the mutant GnFT4 allele comprises a sequence of SEQ ID NO:29 (referred to herein as “GmFT4 Mutant type 1”). Using the genomic sequence of the wild type GmFT4 (SEQ ID NO: 27) as a reference, the mutant allele contains a 5 bp deletion (a deletion from nucleotide position 76 to nucleotide position 80 (relative to the polynucleotide of SEQ ID NO: 27). In some embodiments, the mutant GmFT4 allele comprises a sequence of SEQ ID NO: 31 (referred to herein as “GnFT4 Mutant type 2”), which contains a single nucleotide insertion T in the sequence between nucleotide position 38 and 39. In some embodiments, the mutant GmFT4 allele comprises a sequence that is at least 85%, at least 90%, at least 95%, at least 98%, or at least 99% identical to SEQ ID NO: 29 or 31.

In some embodiments, the genetically modified plant expresses a mutant GmFT4 polypeptide that comprises the polypeptide sequence of SEQ ID NO: 30 or 32. In some embodiments, the genetically modified plant expresses a mutant GmFT4 polypeptide that is at least 85%, at least 90%, at least 95%, at least 98%, or at least 99% identical to SEQ ID NO: 30 or 32.

Under SD conditions, plants in which the genomic sequence of the wild type GmFT4 has been converted to a GmFT4 mutant allele by gene editing mature earlier than a control plant. In some embodiments, these plants mature 2-40 days, e.g., 3-30 days, 4-25 days, 5-20 days, or 5-17 days, earlier relative to a control plant. In some embodiments, these plants mature between 2 to 9 days earlier, for example, 3 to 6 days earlier. See Table 11.

Under LD conditions, plants in which the wild type GmFT4 has been edited to a GmFT4 mutant allele disclosed herein flower and/or mature earlier than a control plant. In some embodiments, these plants flower 2-40 days, e.g., 3-30 days, 4-25 days, 5-20 days, or 5-17 days, earlier relative to a control plant. In some embodiments, these plants flower 4 days earlier. In some embodiments, these plants mature 5-8 days earlier relative to a control plant. See Table 12.

In some embodiments, a soybean plant provided herein comprises a mutant GmFT4 allele comprising a 5-bp deletion and encodes a mutant polypeptide GmFT4 having an amino acid sequence set forth in SEQ ID NO: 30 or 32. In some embodiments, a mutant soybean plant comprising either mutant GmFT4 allele flowers earlier, e.g., about three (3) days earlier, than the control plant (wild type plant) under the LD conditions. In one illustrative embodiment, the mutant soybean plant comprising the mutant GmFT4 allele, matured about three (3) to six (6) days earlier than the control plant (wild type plant) under the SD conditions as shown, for instance, in Example 2 (Tables 11 and 12).

### IV. Polynucleotides and Polypeptides that Confer Delayed Flowering

GmFT5a and GmFT5b are FLOWERING LOCUS T (FT) homologs and flower regulators in soybean. The genomic sequence of the wild type GmFT5a gene is provided as SEQ ID NO: 52 and the coding sequence is SEQ ID NO: 39. The wild type GmFT5a polypeptide comprises a sequence of SEQ ID NO: 40. The genomic sequence of the GmFT5b gene is SEQ ID NO: 54 and the coding sequence is SEQ ID NO: 35. The amino acid sequence of the GmFT5b protein is SEQ ID NO: 36. GmFT5a is highly upregulated under SD conditions and had diurnal expression patterns with the highest expression 4 h after dawn. Under long-day (LD) conditions, expression of GmFT5a was down-regulated and did not follow a diurnal pattern. (Cai et al., Plant Biotechnology J. 2020 January; 18(1): 298-309). There is little information known regarding the expression pattern of GmFT5b in soybean plants. GmFT5b have experienced breeding selection in the process of soybean domestication and breeding. GnFT5b exhibits a high degree of amino acid identify (96.5%) with that of GmFT5a. Ectopic expression experiments in Arabidopsis have demonstrated that GmFT5b can promote flowering (Jiang, B. et al. (2019) Natural variations of FT family genes in soybean varieties covering a wide range of maturity groups, BMC Genomics, 20 (1):230; Wang, Z. et al. (2015) Functional evolution of phosphatidylethanolamine binding proteins in soybean and Arabidopsis, the Plant Cell, 27 (2):323-36; Kong, F. et al. (2010) Two coordinately regulated homologs of FLOWERING LOCUS Tare involved in the control of photoperiodic flowering in soybean, Plant Physiology, 154 (3):1220-31).

The GmFT5a gene or an allele thereof, is in reference to GLYMA_16G044100 (soybase.org). GmFT5a regulates flower development and plant circadian rhythm in soybean. GmFT5a gene is located on chromosome 16. The GmFT5a has a phosphatidylethanolamine-binding domain (Jiang et al., BMC Genomics 20 (1), 230 (2019)).

GmFT5b or an allele thereof, is in reference to GLYMA_19G108200 (soybase.org). Like GmFT5a, GmFT5b also regulates flower development and plant circadian rhythm in soybean. GmFT5a is located on chromosome 19. Like GmFT5a, GmFT5b has a phosphatidylethanolamine-binding domain (Jiang et al., BMC Genomics 20 (1), 230 (2019)).

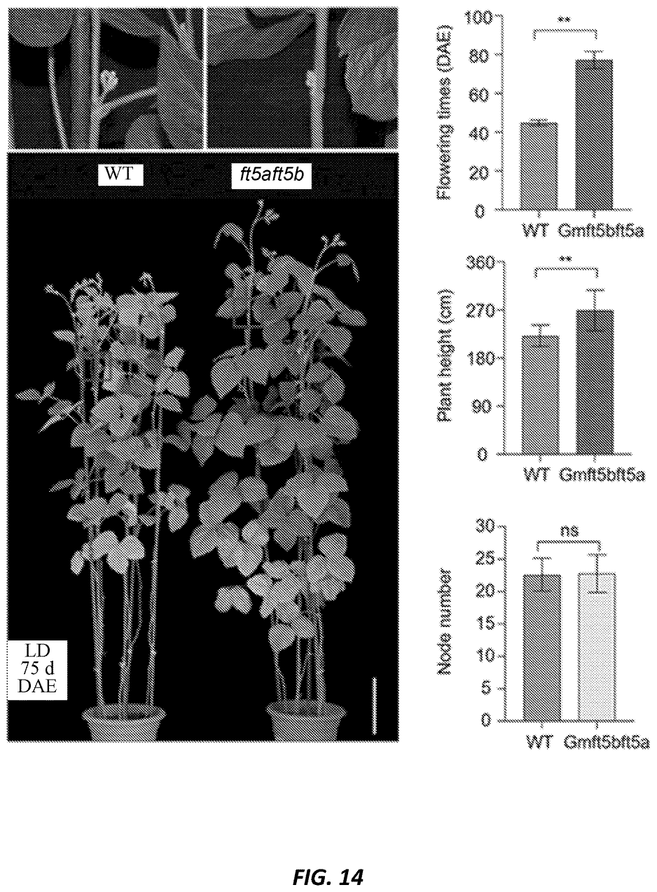

Both Gm FT5a and GmFT5b were reported to promote early flowering in Arabidopsis (Su, Q. et al., Int. J. Mol. Sci. 2022, 23(5), 2497, doi.org/10.3390/ijms23052497; Lee, S. H. et al., Front. Plant Sci., Vol. 12, Art. 613675, 26 Apr. 2021, doi.org/10.3389/fpls.2021.613675). The inventors of this disclosure have found that knocking out GnFT5a and/or GmFT5b individually or together can significantly delay flowering and/or maturity in soybean plants under LD conditions. The effect on flowering and maturity is even more significant when both genes were knocked out in the same plant. In some cases, a soybean plant in which one of GmFT5a or GmFT5b has been knocked out flower or mature 2-50 days, e.g., 4-40 days, 10-30 days, or 5-25 days, later than a control plant under LD conditions. In some cases, a soybean plant in which both GmFT5a and GmFT5b are knocked out flower or mature 25 days, 30 days, 35 days, 40 days, or more, later than a control plant under LD conditions (FIG. 14).

Methods of introducing genomic modification to plants are known, and exemplary approaches are also described in Section V of this application, entitled “methods for producing a plant variety that has altered flowering and/or maturity time”. In some embodiments, the gene editing is performed by CRISPR/Cas9-mediated targeted mutagenesis by using sgRNAs that target a sequence in GmFT5a or GmFT5b. In one exemplary approach, one or more vectors encoding Cas9 and sgRNA containing the target-binding sequence are introduced into a soybean plant. The plants comprising expressing Cas9 and sgRNA can be selected based on the selection marker in the vectors and verified by PCR or sequencing. The resulting mutant GmFT5a or GmFT5b allele can be determined by sequencing. In one illustrative example, editing of GmFT5a uses the reagents in Table 6. In one illustrative example, editing of GmFT5b uses the reagents in in Table 7.

TABLE 6
Regents for knocking out GmFT5a

| | |
|---|---|
| GmFT5a target | AAAGTAAATAATCATGGCACGGG |
| sequence | (SEQ ID NO: 49) |
| DNA oligonucleotide | TTGAAAGTAAATAATCATGGCAC |
| encoding sgRNA-1 | (SEQ ID NO: 50) |
| DNA oligonucleotide | AACGTGCCATGATTATTTACTTT |
| encoding sgRNA-2 | (SEQ ID NO: 51) |

TABLE 7
Regents for knocking out GmFT5b

| | |
|---|---|
| GmFT5b target | GGAGAACCCTCTTGTTATTGGGG |
| sequence | (SEQ ID NO: 46) |
| DNA oligonucleotide | TTGGGAGAACCCTCTTGTTATTG |
| encoding sgRNA-1 | (SEQ ID NO: 47) |
| DNA oligonucleotide | AACCAATAACAAGAGGGTTCTCC |
| encoding sgRNA-2 | (SEQ ID NO: 48) |

In some embodiments, the mutant GmFT5a allele comprises a sequence of SEQ ID NO: 41 and SEQ ID NO: 53. Using the wild type genomic sequence of GnFT5a (SEQ ID NO: 52) as a reference, the mutant allele contains a 1 bp insertion, between nucleotide position 52 and 53 in the polynucleotide having the sequence of SEQ ID NO: 52, resulting in a frameshift-induced premature stop codon in Gmft5a. In some embodiments, the mutant GmFT5a allele comprises a sequence that is at least 85%, at least 90%, at least 95%, at least 98% identical to SEQ ID NO: 41 or 53. In some embodiments, soybean plants that have been modified to comprise the mutant GmFT5a allele flowered significantly later (e.g., about twenty days) than the wild type soybean plant under LD conditions as shown, for instance, in Example 3, particularly Table 16.

In some embodiments, the genetically modified plant expresses a mutant GmFT5a protein having the sequence of SEQ ID NO: 42. In some embodiments, the genetically modified plant expresses a mutant GmFT5a protein that is at least 85%, at least 90%, at least 95%, at least 98%, at least 99% identical to SEQ ID NO: 42.

In some embodiments, the soybean plant provided herein is a double mutant comprising, for example, soybean plant in which the genome has been modified to comprise both a mutant GmFT5a allele and a mutant GmFT5b allele. In particular embodiments, the mutant GmFT5a allele comprises a 1-bp insertion and encodes a mutant polypeptide having an amino acid sequence of SEQ ID NO: 42, and the mutant GmFT5b allele comprises an 8-bp deletion and encodes a mutant polypeptide having an amino acid sequence of SEQ ID NO: 38. In some embodiments, the double mutant soybean plant flowers significantly later than a wild-type soybean plants under LD conditions. In one illustrative example, the double mutant plant flowered about 33 days later and matured at least 34 days later than a wild-type soybean plant as shown, for instance, in Example 3, particularly Table 18.

### V. Methods for Producing a Plant Variety that has Altered Flowering and/or Maturity Time

Provided herein are methods of producing a plant that has altered flowering and/or maturity time. In one aspect, the method can comprise editing the genome of the recipient plant so that the resulting plant comprises a mutant allele encoding one or more mutant polypeptides as described above, e.g., a mutant GmCOL2a polypeptide (SEQ ID NO: 20), a mutant GmCOL2b polypeptide (SEQ ID NO: 26), a mutant GmFT4 polypeptide (SEQ ID NO: 30 or 32), a mutant GmFT5a polypeptide (SEQ ID NO: 42), and/or a mutant GmFT5b polypeptide (SEQ ID NO: 38). In yet another aspect, the method can comprise decreasing the expression level and/or activity of one or more of a mutant GmCOL2a polypeptide (SEQ ID NO: 20), a mutant GmCOL2b polypeptide (SEQ ID NO: 26), a mutant GmFT4 polypeptide (SEQ ID NO: 30 or 32), a mutant GmFT5a polypeptide (SEQ ID NO: 42), and/or a mutant GmFT5b polypeptide (SEQ ID NO: 38) in a recipient plant, for example, by inhibiting promoter activity or by replacing the endogenous promoter with a weaker promoter. In another aspect, the method can comprise breeding a donor plant comprising one or more of the genomic modifications present in one or more of the mutant polypeptides above, e.g., a mutant GmCOL2a polypeptide (SEQ ID NO: 20), a mutant GmCOL2b polypeptide (SEQ ID NO: 26), a mutant GmFT4 polypeptide (SEQ ID NO: 30 or 32), a mutant GmFT5a polypeptide (SEQ ID NO: 42), and/or a mutant GmFT5b polypeptide (SEQ ID NO: 38) with a recipient plant and selecting for incorporation of the corresponding mutant polynucleotide into the recipient plant genome.

### 1. Gene Editing

In some embodiments, the polynucleotide sequences provided herein can be targeted to specific sites within the genome of a recipient plant cell. Such methods include, but are not limited to, meganucleases designed against the plant genomic sequence of interest CRISPR-Cas9, TALENs, and other technologies for precise editing of genomes (Feng, et al. Cell Research 23: 1229-1232, 2013, WO 2013/026740); Cre-lox site-specific recombination; FLP-FRT recombination (Li et al. (2009) Plant Physiol 151:1087-1095); Bxbl-mediated integration (Yau et al. Plant J (2011) 701: 147-166); zinc-finger mediated integration (Wright et al. (2005) Plant J 44:693-705); Cai et al. (2009) Plant Mol Biol 69:699-709); homologous recombination (Lieberman-Lazarovich and Levy (2011) Methods Mol Biol: 51-65); prime editing and transposases (Anzalone, A. et al., Nat Biotechnol. 2020 July; 38(7):824-844); translocation; and inversion.

Various embodiments of the methods described herein use gene editing. In some embodiments, gene editing is used to modify the genome of a plant to produce plants having one or more of the polypeptides that can confer altered flowering and/or maturity time.

In some embodiments, the genomic sequence in the plant to be edited has at least 85%, 90%, 91%, 92%, 93%, 94%, 95%, 96%, 97%, 98%, or 99% identity to the one or more of SEQ ID NO: 15, 21, 27, 52, and/or 54. In some embodiments, the genomic sequence to be edited comprises a nucleic acid sequence set forth in SEQ ID NO: 1, 6, 43, 49, and 46. In some embodiments, the plants that have been modified express one or more of a mutant GmCOL2a polypeptide, a mutant GmCOL2b polypeptide, a mutant GmFT5a polypeptide, a mutant GmFT5b polypeptide, and/or a mutant GmFT4 polypeptide as disclosed above. In particular embodiments, the soybean plant has been edited to express one or more of the following mutant polypeptides: a mutant GmCOL2a polypeptide (SEQ ID NO: 20), a mutant GmCOL2b polypeptide (SEQ ID NO: 26), a mutant GmFT4 polypeptide (SEQ ID NO: 30 or 32), a mutant GmFT5a polypeptide (SEQ ID NO: 42), and/or a mutant GmFT5b polypeptide (SEQ ID NO: 38). In some embodiments, the plant has been edited to express both a mutant GmCOL2a polypeptide (SEQ ID NO: 20) and a mutant GmCOL2b polypeptide (SEQ ID NO: 26). In some embodiments, the plant has been edited to express both a mutant GmFT5a polypeptide (SEQ ID NO: 42) and a mutant GmFT5b polypeptide (SEQ ID NO: 38).

In some embodiments, provided herein are plants transformed with and expressing gene-editing machinery as described above, which, when crossed with a target plant, result in gene editing in the target plant.

In general, gene editing may involve transient, inducible, or constitutive expression of the gene editing components or systems in the target plant. Gene editing may involve genomic integration or episomal presence of the gene editing components or systems.

Gene editing generally refers to the use of a site-directed nuclease (including but not limited to CRISPR/Cas, zinc fingers, meganucleases, and the like) to cut a nucleotide sequence at a desired location. This may be to cause an insertion/deletion (“indel”) mutation, (i.e., “SDN1”), a base edit (i.e., “SDN2”), or allele insertion or replacement (i.e., “SDN3”). SDN2 or SDN3 gene editing may comprise the provision of one or more recombination templates (e.g., in a vector) comprising a gene sequence of interest that can be used for homology directed repair (HDR) within the plant (i.e., to be introduced into the plant genome). In some embodiments, the gene or allele of interest is one that is able to confer to the plant an improved trait, e.g., altered flowering and/or maturity time. The recombination template can be introduced into the plant to be edited either through transformation or through breeding with a donor plant comprising the recombination template. Breaks in the plant genome may be introduced within, upstream, and/or downstream of a target sequence. In some embodiments, a double strand DNA break is made within or near the target sequence locus. In some embodiments, breaks are made upstream and downstream of the target sequence locus, which may lead to its excision from the genome. In some embodiments, one or more single strand DNA breaks (nicks) are made within, upstream, and/or downstream of the target sequence (e.g., using a nickase Cas9 variant). Any of these DNA breaks, as well as those introduced via other methods known to one of skill in the art, may induce HDR. Through HDR, the target sequence is replaced by the sequence of the provided recombination template comprising a polynucleotide of interest. In some embodiments, the target sequences for the gene editing are one of more of a GmCOL2a gene target sequence as set forth in SEQ ID NO: 1, a GmCOL2b gene target sequence as set forth in SEQ ID NO: 6, a GmFT4 gene target sequence as set forth in SEQ ID NO: 43, a GmFT5a gene target sequence as set forth in SEQ ID NO: 49, and/or a GmFT5b gene target sequence as set forth in SEQ ID NO: 46. By designing the system such that one or more single strand or double strand breaks are introduced within, upstream, and/or downstream of the corresponding region in the genome of a plant not comprising the gene sequence of interest, this region can be replaced with the template.

In some embodiments, mutations in the genes of interest described herein may be generated without the use of a recombination template via targeted introduction of DNA double strand breaks. Such breaks may be repaired through the process of non-homologous end joining (NHEJ), which can result in the generation of small insertions or deletions (indels) at the repair site. Such indels may lead to frameshift mutations causing premature stop codons or other types of loss-of-function mutations in the targeted genes.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a“spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cas12a).

The Cas protein as referred to herein, such as but not limited to Cas9, Cas12a (formerly referred to as Cpf1), Cas12b (formerly referred to as C2c1), Cas13a (formerly referred to as C2c2), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is Cas12a, optionally from Acidaminococcus sp., such as Acidaminococcus sp. BV3L6 Cpf1 (AsCas12a) or Lachnospiraceae bacterium Cas12a, such as Lachnospiraceae bacterium MA2020 or Lachnospiraceae bacterium MD2006 (LBCas12a). See U.S. Pat. No. 10,669,540, incorporated herein by reference in its entirety. Alternatively, the Cas12a protein may be from Moraxella bovoculi AAX08_00205 [Mb2Cas12a] or Moraxella bovoculi AAX11_00205 [Mb3Cas12a]. See WO 2017/189308, incorporated herein by reference in its entirety. In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b. Other Cas enzymes are available to a person skilled in the art.

Gene editing methods and compositions are also disclosed in U.S. Pat. Nos. 10,519,456 and 10,285,348 82, the entire content of which is herein incorporated by reference.

The gene-editing machinery (e.g., the DNA modifying enzyme) introduced into the plants can be controlled by any promoter that can drive recombinant gene expression in plants. In some embodiments, the promoter is a constitutive promoter. In some embodiments, the promoter is a tissue-specific promoter, e.g., a pollen-specific promoter or a sperm cell specific promoter, a zygote specific promoter, a root-specific promoter, or a promoter that is highly expressed in sperm, eggs and zygotes (e.g., prOsActin1). Example promoters are disclosed in U.S. Pat. No. 10,519,456, the entire content of which is herein incorporated by reference.

In some embodiments, the guide RNA and the Cas protein (or any other suitable nucleases) may be delivered in DNA form, e.g., in a suitable vector that can be introduced into a yeast cell. Generally, DNA encoding the gRNA is cloned into a vector downstream of a promoter for expression. The sgRNA and Cas may be expressed from the same vector of the system or from different vectors. In some embodiments, the genomic modification of a plant uses a vector PTF101-Cas9 expressing the DNA modification enzyme and one or more pUC57-sgRNA comprising a target sequence of any one of GmCOL2a, GmCOL2b, GmFT5a, GmFT5b, and/or GmFT4. In particular examples, a GmCOL2a gene target sequence as set forth in SEQ ID NO: 1, a GmCOL2b gene target sequence as set forth in SEQ ID NO: 6, a GmFT4 gene target sequence as set forth in SEQ ID NO: 43, a GmFT5a gene target sequence as set forth in SEQ ID NO: 49, and/or a GmFT5b gene target sequence as set forth in SEQ ID NO: 46 can be used. In some embodiments, both the SEQ ID NO: 1 and SEQ ID NO: 6 are used for gene editing to produce a soybean plant comprising both a mutant GmCOL2a allele and a mutant GmCOL2b allele. In some embodiments, both the SEQ ID NO: 49 and SEQ ID NO: 46 are used for gene editing to produce a soybean plant comprising both a mutant GmFT5a allele and a mutant GmFT5b allele. In some embodiments, both the SEQ ID NO: 49 (the GmFT5a target sequence) and SEQ ID NO: 6 (the GmCOL2b target sequence) are used for gene editing to produce a soybean plant comprising both a mutant GmCOL2a allele and a mutant GmCOL2b allele. In some embodiments, the vectors are separately transformed into the target soybean plant to induce gene editing. In some embodiments, the coding sequence for Cas9 and the coding sequence for the sgRNA are ligated into a single vector, which is then transformed into the soybean plant to induce genomic modification. The Cas9 vector and the sgRNA vector typically contains a selection marker, for example, spectinomycin, for identification of transformants comprising the gene editing machinery.

In some embodiments, the target soybean plant is an elite soybean plant, for example, an elite Glycine max plant or an elite Glycine soja plant, and the elite target soybean plant can be edited using the methods described above to express one or more of the following mutant polypeptides: a mutant GmCOL2a polypeptide (SEQ ID NO: 20), a mutant GmCOL2b polypeptide (SEQ ID NO: 26), a mutant GmFT4 polypeptide (SEQ ID NO: 30 or 32), a mutant GmFT5a polypeptide (SEQ ID NO: 42), and/or a mutant GmFT5b polypeptide (SEQ ID NO: 38).

In some embodiments, the target soybean plant, (optionally an elite soybean target plant) can be edited using the methods described above to express one or more of the following mutant polypeptides: a mutant GmCOL2a polypeptide that is at least 85%, at least 90%, at least 95%, at least 98%, or at least 99% identical to SEQ ID NO: 20, a mutant GmCOL2b polypeptide that is at least 85%, at least 90%, at least 95%, at least 98%, or at least 99% identical to SEQ ID NO: 26), a mutant GmFT4 polypeptide that is at least 85%, at least 90%, at least 95%, at least 98%, or at least 99% identical to SEQ ID NO: 30 or 32, a mutant GmFT5a polypeptide that is at least 85%, at least 90%, at least 95%, at least 98%, or at least 99% identical to SEQ ID NO: 42, and/or a mutant GmFT5b polypeptide that is at least 85%, at least 90%, at least 95%, at least 98%, or at least 99% identical to SEQ ID NO: 38.

In some embodiments, the method of introducing desired genomic modifications comprises using a first soybean plant expressing a DNA modification enzyme and at least one optional guide nucleic acid as described above to pollinate a target plant comprising genomic DNA to be edited.

### 2. Crossing

In some embodiments, the method comprises crossing a donor plant comprising genomic modification disclosed herein with a recipient plant, and the genomic modification is able to confer altered flowering and/or maturity time in the recipient plant. As used herein, the terms “crossing” and “breeding” refer to the fusion of gametes to produce progeny (e.g., by fertilization, such as to produce seed by pollination in plants). In some embodiments, a “cross,” “breeding,” or “cross-fertilization” is fertilization of one individual by another (e.g., cross-pollination in plants). The plant disclosed herein may be a whole plant, or may be a plant cell, seed, or tissue, or a plant part such as leaf, stem, pollen, or cell that can be cultivated into a whole plant. In some embodiments, the donor plant or the recipient plant is an elite soybean plant, for example, an elite Glycine max plant or an elite Glycine soja plant.

In some embodiments, a donor plant that has been edited to express one or more of the following mutant polypeptides: a mutant GmCOL2a polypeptide (SEQ ID NO: 20), a mutant GmCOL2b polypeptide (SEQ ID NO: 26), a mutant GmFT4 polypeptide (SEQ ID NO: 30 or 32), a mutant GmFT5a polypeptide (SEQ ID NO: 42) and/or a mutant GmFT5b polypeptide (SEQ ID NO: 38), can be crossed with an elite recipient soybean plant to produce progeny plants comprising such mutant alleles.

In some embodiments, the donor plant that has been edited to express one or more of a mutant GmCOL2a polypeptide that is at least 85%, at least 90%, at least 95%, at least 98%, or at least 99% identical to SEQ ID NO: 20, a mutant GmCOL2b polypeptide that is at least 85%, at least 90%, at least 95%, at least 98%, or at least 99% identical to SEQ ID NO: 26), a mutant GmFT4 polypeptide that is at least 85%, at least 90%, at least 95%, at least 98%, or at least 99% identical to SEQ ID NO: 30 or 32, a mutant GmFT5a polypeptide that is at least 85%, at least 90%, at least 95%, at least 98%, or at least 99% identical to SEQ ID NO: 42, and/or a mutant GmFT5b polypeptide that is at least 85%, at least 90%, at least 95%, at least 98%, or at least 99% identical to SEQ ID NO: 38. And said donor plant can be crossed with an elite recipient soybean plant to produce progeny plants comprising such mutant alleles

In some embodiments, a progeny plant created by the crossing or breeding process is repeatedly crossed back to one of its parents through a process referred to herein as “backcrossing”. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted Backcrossing: A Practical Example, in Techniques et Utilisations des Marqueurs Moleculaires Les Colloques, Vol. 72, pp. 45-56; and Openshaw et al. (1994) Marker-assisted Selection in Backcross Breeding, in Proceedings of the Symposium “Analysis of Molecular Marker Data,” Joint Plant Breeding Symposia Series, American Society for Horticultural Science/Crop Science of America, Corvallis, Oregon, pp. 41-43. The initial cross gives rise to the F1 generation. The term “BC1” typically refers to the second use of the recurrent parent, “BC2” refers to the third use of the recurrent parent, and so on.

### 3. Gene Regulation and Silencing

In some approaches, the method of conferring altered flowering and/or maturity time involves repressing transcription of the one or more of the wild type alleles of GmCOL2a, GmCOL2b, GmFT5a, GmFT5b, and/or GmFT4. In some embodiments, the method comprises delivering a transcriptional repressor that can bind to a transcriptional regulatory region of any one of the GmCOL2a, GmCOL2b, GmFT5a, GmFT5b, and/or GmFT4 genes in the plant, thereby inhibiting transcription and reducing the expression of the wild type polypeptides. In some embodiments, the expression of one or more the wild type polypeptides GmCOL2a, GmCOL2b, GmFT5a, GmFT5b, and/or GmFT4 is reduced by at least 50%, at least 60%, at least 70%, at least 80%, at least 90% or at least 95% as compared to a control plant.

In some approaches the method of conferring altered flowering and/or maturity time involves reducing protein translation efficiency, for example by using codons that are not optimized for expression in soybean plants.

In some approaches the method of conferring altered flowering and/or maturity time involves mutagenizing the transcriptional regulatory region of one or more of the genes disclosed herein, e.g., GmCOL2a (SEQ ID NO: 15), GmCOL2b (SEQ ID NO: 21), GmFT4 (SEQ ID NO: 27), GmFT5a (SEQ ID NO: 52), and GmFT5b (SEQ ID NO: 54) to modulate transcriptional level of the polypeptides, thereby altering flowering and/or maturity time. A gene regulatory region as used herein is the region of a gene where RNA Polymerase and other accessory transcription modulator proteins bind and interact to control RNA synthesis. Although the promoter is an integral part of the regulatory region, this region may also contain binding sites for proteins that function in either a positive or a negative modulating fashion and various nucleotide sequence features, such as attenuators, may contribute to regulation of transcription. In one example, deletion of one or more of these functional sequences can be made. In one example, a deletion can be made to eliminate the normal start codon(s) in the gene. In another example, one or more point mutations can be introduced to alter the start codon for the RNA transcript and, optionally, one or more point mutations may be introduced to alter any other in-frame AUG (methionine codon) near the normal initiation codon for the transcript.

In some approaches, the method of conferring altered flowering and/or maturity time involves silencing the expression of the wild type GmCOL2a, GmCOL2b, GnFT4, GnFT5a, and/or GmFT5b gene by-using expression cassettes that transcribe inhibitory RNA molecules (or fragments thereof) that inhibit wild type gene expression or activity in a plant cell. Nonlimiting examples of the inhibitory RNA molecules include short interfering RNA (siRNA) and microRNA (miRNA)), anti-sense RNA, and the like.

RNAi (e.g., siRNA, miRNA) function by base-pairing to complementary RNA or DNA target sequences. When bound to RNA, the inhibitory RNA molecules trigger either RNA cleavage or translational inhibition of the target sequence. When bound to DNA target sequences, it is thought that inhibitory RNAs can mediate DNA methylation of the target sequence. MicroRNAs (miRNAs) are noncoding RNAs of about 19 to about 24 nucleotides in length that are processed from longer precursor transcripts that form stable hairpin structures. Any approaches, regardless of the specific mechanism, that can result in inhibition of gene expression of one or more of the wild type GmCOL2a, GmCOL2b, GmFT4, GmFT5a, and/or GmFT5b genes can be used in the method disclosed herein. Other methods that can reduce transcription and/or translation of one or more of the wild type GmCOL2a, GmCOL2b, GmFT4, GmFT5a, or GmFT5b polypeptides can also be used to alter flowering and/or maturity time.

### VI. Plants, Plant Cells and Plant Parts

Although soybean plants are used to exemplify the composition and methods throughout the application, any plant species can be edited to knock out the one or more a genomic DNA in the plant to confer altered flowering and/or maturity time. These plant species include, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Glycine (soybean or soya bean) is a genus in the bean family Fabaceae. The Glycine plants can be Glycine arenaria, Glycine argyrea, Glycine cyrtoloba, Glycine canescens, Glycine clandestine, Glycine curvata, Glycine falcata, Glycine latifolia, Glycine microphylla, Glycine pescadrensis, Glycine stenophita, Glycine syndetica, Glycine soja Seib. Et Zucc., Glycine max (L.) Merrill., Glycine tabacina, or Glycine tomentella.

In some embodiments, the soybean plant is an elite soybean plant, for example, an elite Glycine max plant or an elite Glycine soja plant. In some embodiments, the elite soybean plant expresses one or more of the following mutant polypeptides: a mutant GmCOL2a polypeptide (SEQ ID NO: 20), a mutant GmCOL2b polypeptide (SEQ ID NO: 26), a mutant GmFT4 polypeptide (SEQ ID NO: 30 or 32), a mutant GmFT5a polypeptide (SEQ ID NO: 42), and/or a mutant GmFT5b polypeptide (SEQ ID NO: 38). In some embodiments, the elite soybean plant expresses one or more of a mutant GmCOL2a polypeptide that is at least 85%, at least 90%, at least 95%, at least 98%, or at least 99% identical to SEQ ID NO: 20, a mutant GmCOL2b polypeptide that is at least 85%, at least 90%, at least 95%, at least 98%, or at least 99% identical to SEQ ID NO: 26), a mutant GmFT4 polypeptide that is at least 85%, at least 90%, at least 95%, at least 98%, or at least 99% identical to SEQ ID NO: 30 or 32, a mutant GmFT5a polypeptide that is at least 85%, at least 90%, at least 95%, at least 98%, or at least 99% identical to SEQ ID NO: 42, and/or a mutant GmFT5b polypeptide that is at least 85%, at least 90%, at least 95%, at least 98%, or at least 99% identical to SEQ ID NO: 38.

Plants produced as described above can be propagated to produce progeny plants, and the progeny plants that have stably incorporated into its genome the genomic modification disclosed herein which confer the altered flowering and/or maturity time can be selected. These progeny plants can be further propagated if desired. The term “progeny,” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

In some embodiments the modified plant or its progeny plant comprises a homozygous mutant allele of the genes disclosed herein. In some embodiments, the mutant allele does not occur in nature in the plant. Plants comprising a homozygous mutant allele disclosed herein can be readily selected by methods well known in the art, for example, PCR or sequencing.

In some embodiments, a plant cell, seed, or plant part or harvest product can be obtained from the plant produced as above and the plant cell, seed, or plant part can be screened using methods disclosed above for the evidence of stable incorporation of the polynucleotide. As used herein, the term “plant part” indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, zygotes, leaves, embryos, roots, root tips, anthers, flowers, flower parts, fruits, stems, shoots, cuttings, and seeds; as well as pollen, ovules, egg cells, zygotes, leaves, embryos, roots, root tips, anthers, flowers, flower parts, fruits, stems, shoots, cuttings, scions, rootstocks, seeds, protoplasts, calli, and the like.

In some embodiments, plant products can be harvested from the plant disclosed above and processed to produce processed products, such as flour, soy meal, oil, starch, and the like. These processed products are also within the scope of this invention provided that they comprise a polynucleotide or polypeptide or variant thereof disclosed herein. Other soybean plant products include but are not limited to protein concentrate, protein isolate, soybean hulls, meal, flower, oil and the whole soybean itself.

### EXEMPLARY EMBODIMENTS

Embodiment 1 is a plant having a genomic modification, wherein the genomic modification comprises a knock out of one or more of the following genes: GmCOL2a; GmCOL2b; GmFT5a; GmFT5b, or GmFT4, wherein the plant has an altered flowering time and/or maturity time relative to a control plant not comprising the genomic modification.

Embodiment 2 is the plant of embodiment 1, wherein the genomic modification results in decreased expression and/or activity of a polypeptide encoded by the one or more genes, and the decreased expression and/or activity of the polypeptide results in the altered flowering time and/or maturity time under long day (LD) and/or short day (SD) conditions.

Embodiment 3 is the plant of embodiment 1, wherein the genomic modification is non-natural to the plant.

Embodiment 4 is the plant of embodiment 3, wherein said genomic modification comprises a deletion, an insertion, or a substitution in a genomic DNA sequence of said one or more genes.

Embodiment 5 is the plant of embodiment(s) 3 or 4, wherein the genomic DNA sequence for the one or more genes comprises: (a) at least 85%, 90%, 91%, 92%, 93%, 94%, 95%, 96%, 97%, 98%, or 99% identity to at least one of SEQ ID NO: 15, 21, 27, 52 or 54, (b) at least 85%, 90%, 91%, 92%, 93%, 94%, 95%, 96%, 97%, 98%, 99%, identity to at least one of the SEQ ID NO: 16, 22, 56, 35, or 39, and/or (c) a nucleic acid sequences set forth in SEQ ID NO: 1, 6, 43, 49, and 46.

Embodiment 6 is the plant of any one of embodiments 1-5, wherein the genomic modification is accomplished through CRISPR, TALEN, or meganucleases.

Embodiment 7 is the plant of embodiment 6, wherein the genomic modification is accomplished through Cas12a-mediated gene editing.

Embodiment 8 is the plant of embodiment 7, wherein the Cas12a-mediated gene edit employs a gRNA having a target sequence comprising one or more of SEQ ID NOS: 1, 6, 43, 49, or 46.

Embodiment 9 is the plant of embodiment 1, wherein the genomic modification of the one or more genes results in the plant expressing one or more of a mutant GmCOL2a polypeptide; a mutant GmCOL2b polypeptide; a mutant GmFT5a polypeptide; a mutant GmFT5b polypeptide; and/or a mutant GmFT4 polypeptide.

Embodiment 10 is the plant of embodiment 1, wherein the genomic modification of the one or more genes results in the plant expressing one or more of a mutant GmCOL2a allele; a mutant GmCOL2b allele; a mutant GmFT5a allele; a mutant GmFT5b allele; and/or a mutant GmFT4 allele.

Embodiment 11 is the plant of any one of embodiments 1-10, wherein the genomic modification results in a decrease in expression and/or activity of a polypeptide comprising: (a) an amino acid sequence comprising at least 85%, 90%, 91%, 92%, 93%, 94, 95%, 96%, 97%, 98%, or 99% identity to at least one of SEQ ID NOS: 17, 23, 28, 36, or 40, and/or (b) an amino acid sequence set forth in at least one of SEQ ID NOS: 17, 23, 28, 36, or 40.

Embodiment 12 is the plant of embodiments 1 or 9, wherein the genomic modification results in at least 80% decrease in expression of one or more of wild type GmCOL2a polypeptide (SEQ ID NO: 17), wild type GmCOL2b polypeptide (SEQ ID NO: 23), wild type GmFT5a polypeptide (SEQ ID NO: 40), wild type GmFT5b polypeptide (SEQ ID NO: 36), or wild type GmFT4 polypeptide (SEQ ID NO: 28) relative to the control plant not comprising the genomic modification.

Embodiment 13 is the plant of embodiment 9 or 12, wherein the mutant GmCOL2a polypeptide, the mutant GmCOL2b polypeptide, the mutant GmFT5a polypeptide, the mutant GmFT5b polypeptide, or the mutant GmFT4 polypeptide share less than 20% identity with corresponding wild type polypeptides.

Embodiment 14 is the plant of embodiment 9 or 12, wherein the mutant GmCOL2a polypeptide, the mutant GmCOL2b polypeptide, the mutant GmFT5a polypeptide, the mutant GmFT5b polypeptide, or the mutant GmFT4 polypeptide are non-functional polypeptides.

Embodiment 15 is the plant of any one of embodiments 9-14, wherein the plant expresses a mutant GmCOL2a polypeptide comprising: (a) an amino acid sequence that is at least 85% identical to SEQ ID NO: 20, or (b) an amino acid sequence as set forth in SEQ ID NO: 20.

Embodiment 16 is the plant of embodiment 15, wherein the mutant GmCOL2a polypeptide is encoded by a sequence that is at least 85% identical to SEQ ID NO:18 or 19.

Embodiment 17 is the plant of any one of embodiments 9-14, wherein the mutant GmCOL2b polypeptide comprises an amino acid sequence that has at least 85% identity to SEQ ID NO: 26, or wherein the mutant GmCOL2b polypeptide is encoded by a nucleic acid sequence that has at least 85% identity to SEQ ID NO: 24 or 25.

Embodiment 18 is the plant of any one of embodiments 9-14, wherein the mutant GmFT5a polypeptide comprises an amino acid sequence that has at least 85% identity to SEQ ID NO: 42, or wherein the mutant GmFT5a polypeptide is encoded by a nucleic acid sequence that has at least 85% identity to SEQ ID NO: 41 or SEQ ID NO: 53.

Embodiment 19 is the plant of any one of embodiments 9-14, wherein the mutant GmFT5b polypeptide comprises an amino acid sequence that has at least 85% identity to SEQ ID NO: 38, or wherein the mutant GmFT5b polypeptide is encoded by a nucleic acid sequence that has at least 85% identity to SEQ ID NO: 37 or SEQ ID NO: 55.

Embodiment 20 is the plant of any one of embodiments 9-14, wherein the mutant GmFT4 polypeptide comprises an amino acid sequence that has at least 85% identity to SEQ ID NO: 32 or 30, or wherein the mutant GmFT4 polypeptide is encoded by a nucleic acid sequence that has at least 85% identity to SEQ ID NO: 31 or 29.

Embodiment 21 is the plant of any one of embodiments 1-20, wherein the plant is knocked out for one or more of the GmCOL2a gene, the GmCOL2b gene, or the GmFT4 gene, and wherein the plant flowers and/or matures earlier relative to a control plant not comprising the genomic modification under LD conditions.

Embodiment 22 is the plant of embodiment 21, wherein the genetically modified plant flowers and/or matures at least 2 days earlier than the control plant under LD conditions.

Embodiment 23 is the plant of embodiment 21, wherein the genetically modified plant flowers and/or matures 2-40 days earlier than the control plant under LD conditions.

Embodiment 24 is the plant of any one of embodiments 1-22, wherein the plant is knocked out for both the GmCOL2a gene and the GmCOL2b gene, and wherein the plant flowers and/or matures 2-40 days earlier than a control plant under LD conditions.

Embodiment 25 is the plant of any one of embodiments 1-22, wherein the plant is knocked out for the GmFT4 gene, and wherein plant flowers and/or matures 2-40 days earlier than a control plant under LD conditions and matures 2-40 days earlier than a control plant under SD conditions.

Embodiment 26 is the plant of any one of embodiments 1-20, wherein the plant is knocked out for GmFT5a or GmFT5b, or both GmFT5a and GmFT5b, and wherein the plant flowers and/or matures later relative to a control plant not comprising the genomic modification under LD conditions.

Embodiment 27 is the plant of embodiment 26, wherein the genetically modified plant flowers and/or matures at least 2 days later than the control plant under LD conditions.

Embodiment 28 is the plant of embodiment 27, wherein the genetically modified plant flowers and/or matures 2-40 days later than a control plant under LD conditions.

Embodiment 29 is the plant of any one of embodiments 1-22, wherein the plant is knocked out for both GmFT5a and GmFT5b, and wherein the genetically modified plant flowers and/or matures 30-70 days later than a control plant under LD condition.

Embodiment 30 is the plant of any one of embodiments 1-29, wherein the plant is a dicot plant.

Embodiment 31 is the plant of embodiment 30, wherein the dicot plant is a soybean plant, and optionally wherein the soybean plant is an alite soybean plant.

Embodiment 32 is a plant cell, seed, or plant part derived from the plant of any one of embodiment(s)s 9-31, wherein the plant cell, seed, or plant part expresses one or more of the mutant GmCOL2a polypeptide, the mutant GmCOL2b polypeptide, the mutant GmFT5a polypeptide, the mutant GmFT5b polypeptide, or the mutant GmFT4 polypeptide.

Embodiment 33 is a harvested product derived from the plant of any one of embodiments 9-31, or the plant cell, seed, or plant part of embodiment 32, wherein the harvested product expresses one or more of the mutant GmCOL2a polypeptide, the mutant GmCOL2b polypeptide, the mutant GmFT5a polypeptide, the mutant GmFT5b polypeptide, or the mutant GmFT4 polypeptide.

Embodiment 34 is a processed product derived from the harvest product of embodiment 33, wherein the altered flowering of the genetically modified plant includes fewer days between a VE stage and an R1 stage of the modified plant relative to the control plant.

Embodiment 35 is a plant expressing both a mutant GmCOL2a polypeptide and a mutant GmCOL2b polypeptide, wherein mutant GmCOL2a polypeptide comprises: (a) an amino acid sequence that is at least 85% identical to SEQ ID NO: 20, or (b) an amino acid sequence as set forth in SEQ ID NO: 20, and wherein the mutant GmCOL2b polypeptide comprises an amino acid sequence that has at least 85% identity to SEQ ID NO: 26, or (b) an amino acid sequence as set forth in SEQ ID NO: 26.

Embodiment 36 is a plant expressing both a mutant GmFT5a polypeptide and a mutant GmFT5b polypeptide, wherein mutant GmFT5a polypeptide comprises: (a) an amino acid sequence that is at least 85% identical to SEQ ID NO: 42, or (b) an amino acid sequence as set forth in SEQ ID NO: 42, and wherein the mutant GmFT5b polypeptide comprises an amino acid sequence that has at least 85% identity to SEQ ID NO: 38, or (b) an amino acid sequence as set forth in SEQ ID NO: 38.

Embodiment 37 is a method of altering flowering time and/or maturity time in a soybean plant, the method comprising, editing in the genome of a soybean plant one or more of the following genes: GmCOL2a GmCOL2b, GmFT5a, GmFT5b, or GmFT4, thereby forming a modified soybean plant, wherein the modified soybean plant has a flowering time and/or maturity time that is altered relative to a control plant not comprising the editing in one or more of the genes.

Embodiment 38 is the method of embodiment 37, wherein the editing includes knocking out of the one or more genes resulting in the modified soybean plant expressing one or more of: a mutant GmCOL2a polypeptide, a mutant GmCOL2b polypeptide, a mutant GmFT5a polypeptide, a mutant GmFT5b polypeptide, or a mutant GmFT4 polypeptide.

Embodiment 39 is the method of embodiment 38, wherein the mutant GmCOL2a polypeptide, the mutant GmCOL2b polypeptide, the mutant GmFT5a polypeptide, the mutant GmFT5b polypeptide, or the mutant GmFT4 polypeptide share less than 20% identity with a corresponding wild-type polypeptide.

Embodiment 40 is the method of embodiment 38 or 39, wherein the mutant GmCOL2a polypeptide, the mutant GmCOL2b polypeptide, the mutant GmFT5a polypeptide, the mutant GmFT5b polypeptide, or the mutant GmFT4 polypeptide each is a non-functional polypeptide.

Embodiment 41 is the method of embodiment 38 or 39, wherein the mutant GmCOL2a polypeptide comprises SEQ ID NO: 20, the mutant GmCOL2b polypeptide comprises SEQ ID NO: 26, the mutant GmFT5a polypeptide comprises SEQ ID NO: 42, the mutant GmFT5b polypeptide comprises SEQ ID NO: 38, or the mutant GmFT4 polypeptide comprises SEQ ID NO: 30 or 32.

Embodiment 42 is the method of any one of embodiments 38-40, wherein knocking out of the GmCOL2a gene, the GmCOL2b gene, the GmFT5a gene, the GmFT5b gene, or the GmFT4 gene is performed through gene editing using site-directed nucleases.

Embodiment 43 is the method of embodiment 42, wherein the site-directed nuclease is selected from the group consisting of Cas 12 nuclease, a meganuclease, a zinc-finger nuclease, or a transcription-activator like effector nuclease.

Embodiment 44 is the method of any one of embodiments 38-43, wherein the knocking out of the GmCOL2a gene, the GmCOL2b gene, the GmFT5a gene, the GmFT5b gene, or the GmFT4 gene is performed using Cas nuclease and a guide RNA comprising a nucleotide sequence corresponding to a target sequence in one or more of GmCOL2a gene, the GmCOL2b gene, the GmFT5a gene, the GmFT5b gene, or the GmFT4 gene, respectively.

Embodiment 45 is the method of embodiment 44, wherein the target sequence in the GmCOL2a gene comprises SEQ ID NO: 1.

Embodiment 46 is the method of embodiment 44 or 45, wherein the guide RNA for gene editing of the GmCOL2a gene is encoded by SEQ ID NO: 2 or 3.

Embodiment 47 is the method of embodiment 44, wherein the target sequence in the GmCOL2b gene comprises SEQ ID NO: 6.

Embodiment 48 is the method of embodiment 44 or 47, wherein the guide RNA for genetically modifying the GmCOL2b gene is encoded by SEQ ID NO: 7 or 8.

Embodiment 49 is the method of embodiment 44, wherein the target sequence in the GmFT5a gene comprises SEQ ID NO: 49.

Embodiment 50 is the method of embodiment 44 or 49, wherein the guide RNA for gene editing the GmFT5a gene is encoded by SEQ ID NO: 50 or 51.

Embodiment 51 is the method of embodiment 44, wherein the target sequence in the GmFT5b gene comprises SEQ ID NO: 46.

Embodiment 52 is the method of embodiment 44 or 51, wherein the guide RNA for gene editing the GmFT5b gene is encoded by SEQ ID NO: 47 or 48.

Embodiment 53 is the method of embodiment 44, wherein the target sequence in the GmFT4 gene comprises SEQ ID NO: 43.

Embodiment 54 is the method of embodiment 44 or 53, wherein the guide RNA for gene editing of the GmFT4 gene is encoded by SEQ ID NO: 44 or 45.

Embodiment 55 is the method of any one of embodiments 37-54, wherein the editing includes knocking out one or more of GmCOL2a, GmCOL2b, or GmFT4, wherein the method further comprises detecting accelerated flowering and/or maturity in the modified soybean plant as compared to a control plant under LD conditions.

Embodiment 56 is the method of embodiment 55, wherein the LD condition is 16 h light/8 h dark in a 24-hour period.

Embodiment 57 is the method of embodiment 55, wherein the accelerated flowering and/or maturity is at least 2 days earlier, at least 4 days earlier, at least 5 days earlier, at least 6 days earlier, or at least 7 days earlier as compared to a control plant grown under LD conditions.

Embodiment 58 is the method of any one of embodiments 37-54, wherein the editing includes knocking out one or both of GmFT5a and GmFT5b, wherein the method further comprises detecting delayed flowering and/or maturity in the modified soybean plant as compared to a control plant under LD conditions, wherein the detecting delayed flowering is based on counting the number of days elapsed between the VE stage and R1 stage, and wherein detecting delayed maturity is based on counting the number of days between the VE stage and the R7 stage.

Embodiment 59 is the method of embodiment 60, wherein the delayed flowering is at least 2 days later, at least 4 days later, at least 5 days later, at least 6 days later, or at least 7 days later as compared to a control plant grown under LD conditions.

Embodiment 60 is a modified soybean plant produced using the method of any one of embodiments 37-59.

Embodiment 61 is a plant cell, seed, or plant part derived from the modified soybean plant of embodiment 60.

Embodiment 62 is a method of breeding, comprising: crossing the plant of any one of embodiments 10-31 with a different plant not comprising the one or more mutant alleles; wherein both plants are soybean plants, and selecting a progeny plant having altered flowering and/or maturity time.

Embodiment 63 is the method of embodiment(s) 62, wherein the different plant is an elite soybean plant.

Embodiment 64 is a plant comprising a genomic modification that results in decreased expression and/or activity of a polypeptide comprising: (a) an amino acid sequence comprising at least 85%, 90%, 91%, 92%, 93%, 94, 95%, 96%, 97%, 98%, or 99% identity to at least one of SEQ ID NOS: 7, 23, 28, 36, or 40, or (b) an amino acid sequence as set forth in at least one of SEQ ID NOS: 17, 23, 28, 36, or 40, wherein the modification is heterologous to the plant and the decreased expression and/or activity in the plant results in the plant having an altered flowering and/or maturity time compared to a control plant not comprising the genomic modification, and wherein the genomic modification is introduced via genome editing.

Embodiment 65 is a modified soybean plant, or plant part thereof, comprising one or more non-naturally occurring mutant alleles at one or more loci, wherein the non-naturally occurring mutant allele is introduced via genomic modification using a site directed nuclease, wherein the one or more loci comprise GmFT4a, GmFT5a, GmFT5b, GmCOL2a, or GmCOL2b, and wherein the one or more mutant alleles result in an altered flowering and/or maturity time of the plant relative to a control plant not comprising the mutant allele.

Embodiment 66 is the modified soybean plant, or plant part thereof, of embodiment 65, wherein said non-naturally occurring mutant allele is a homozygous mutant allele.

Embodiment 67 is the modified soybean plant, or plant part thereof, of embodiment 65, comprising a non-naturally occurring mutant allele at each of the GmFT5a locus and the GmFT5b locus, wherein both of said loci comprise homozygous mutant alleles.

Embodiment 68 is the modified soybean plant, or plant part thereof, of embodiment 65, comprising a non-naturally occurring mutant allele at each of the GmCOL2a locus and the GmCOL2b locus, wherein both of said loci comprise homozygous mutant alleles.

Embodiment 69 is the modified soybean plant, or plant part thereof, of embodiment 65, comprising a non-naturally occurring homozygous mutant allele at the GmFT4a locus.

Embodiment 70 is the modified soybean plant, or plant part thereof, of any one of embodiment(s)s 65-69, wherein said mutant allele exhibits a reduction of expression or activity relative to an unmodified, wild-type gene allele and wherein the mutant allele results in the modified soybean plant having the altered flowering and/or maturity time when grown under LD conditions.

Embodiment 71 is the modified soybean plant, or plant part thereof, of any one of embodiments 65-70, wherein said mutant allele results in the modified soybean plant having an accelerated flowering and/or maturity relative to the control plant when grown under LD conditions.

Embodiment 72 is the modified soybean plant, or plant part thereof, of any one of embodiments 65-71, wherein at least one of the mutant alleles comprises a nonsense mutation, an in-frame deletion mutation, a missense mutation, a frameshift mutation, a splice-site mutation, or any combination thereof.

Embodiment 73 is the modified soybean plant, or plant part thereof, of any one of embodiments 65-72, wherein at least one of the mutant alleles encodes a protein truncation, a non-functional protein, a protein with reduced function relative to a protein expressed by the corresponding wild type allele, and/or wherein at least one of the mutant alleles comprises a premature stop codon, a frame-shift mutation, and an in-frame deletion relative to the corresponding wild type allele.

### EXAMPLES

The provided methods and compositions will be further described in the following examples, which do not limit the scope of the methods and compositions of matter described in the claims.

### Example 1. Targeted Mutagenesis of GmCOL2a and GmCOL2b Accelerates Flowering in Soybean

### 1.1 SgRNA Design and Construction of the Gene Editing Vector for GmCOL2a and GmCOL2b

In this study, the vector pUC57-SgRNA (SEQ ID NO: 13) was used for sgRNA construction and expression. The vector was linearized by the restriction enzyme NHeI and BbsI, and then the fragment about 3201 bp was extracted using Zymoclean™ Gel DNA Recovery Kit (D4008).

The sequence and other information for the analyzed soybean endogenous gene GmCOL2a (Glyma.13G050300) were downloaded from the Phytozome website (phytozome-next.jgi.doe.gov/info/Gmax_Wm82_a2 v1). We designed the sgRNA using the web tool CRISPR-P (crispr.hzau.edu.cn/CRISPR/) and chose the target sequence GmCOL2a-SP1: 5′-TTGGTGGCAGCACCGGCACCTGG-3′ (SEQ ID NO: 1) for GmCOL2a. We then synthesized the primers GmCOL2a-Cas9-F: 5′-TCGAAGTAGTGATTGTTGGTGGCAGCACCGGCACCGTTTTAGAGCTAGAA-3′ (SEQ ID NO: 2) and GmCOL2a-Cas9-R: 5′-TTCTAGCTCTAAAACGGTGCCGGTGCTGCCACCAACAATCACTACTTCGA-3′ (SEQ ID NO: 3) from TSINGKE (Beijing).

The sequence and other information for the analysed soybean endogenous gene GmCOL2b (Glyma.19G039000) were downloaded from the Phytozome website (phytozome-next.jgi.doe.gov/info/Gmax_Wm82_a2_v1). We designed the sgRNA using the web tool CRISPR-P (http://crispr.hzau.edu.cn/CRISPR/) and choose the target sequence GmCOL2b-SP1: 5′-GCAGCAACACTGGCACCACCTGG-3′ (SEQ ID NO: 6) for GnCOL2b. We then synthesized the primers GmCOL2b-Cas9-F: 5′-TCGAAGTAGTGATTGGCAGCAACACTGGCACCACCGTTTTAGAGCTAGAA-3′ (SEQ ID NO: 7) and GmCOL2b-Cas9-R: 5′-TTCTAGCTCTAAAACGGTGGTGCCAGTGTTGCTGCCAATCACTACTTCGA-3′(SEQ ID NO: 8) from TSINGKE (Beijing).

This pair of DNA oligos were then annealed (Each 5 μL of the 10 M GmCOL2a-Cas9-F/R and 15 μL ddH₂O were mixed well. The mixture was then placed at 95° C. for 3 min. After that, the temperature is slowly cooled down to 16° C. by −1° C./20 s) to generate a dimer, which was subsequently integrated into the linearized pUC57-SgRNA vector using ClonExpress Ultra One Step Cloning Kit (Vazyme, C115-01).

The ligation product from last step was transformed into E. coli DH5a competent cells, then incubated on ice for 30 min, heated shock at 42° C. for 90 s in a water bath, and then incubated on ice for 2 min, added 700 μL LB liquid medium (10 g/L tryptone, 5 g/L yeast extract and 10 g/L NaCl), and incubated at 37° C. with shaking at 180 rpm for 1 h. We then spread all bacteria on LB plates (10 g/L tryptone, 5 g/L yeast extract, 10 g/L NaCl, and 15 g/L agar) with 100 mg/mL ampicillin and incubated them overnight at 37° C.

Some mono-clones were then sequenced by TSINGKE (Beijing) using the primer pSgRNA-CX: 5′-CGCCAGGGTTTTCCCAGTCACGAC-3′ (SEQ ID NO: 65). The consequent construct was purified using the TIANprep Rapid Mini Plasmid Kit (TIANGEN, DP103-200) for subsequent use.

The vector PTF101-Cas9 was used for Cas9 expression. In this vector, the bar gene was used as an herbicide resistance marker. Both the two plasmids PTF101-Cas9 and pUC57-SgRNA containing target sequence of GmCOL2a were then cleaved by enzyme digestion using PacI and PmeI. The two linearized fragments were then integrated by T4 DNA Ligase.

The ligation product from last step was transformed into E. coli DH5a competent cells, and then spread on LB plates with 50 mg/mL spectinomycin and incubated at 37° C. overnight. Some mono-clones were then sequenced by TSINGKE (Beijing) using the primer pCas9-TYJC: 5′-TGGGAATCTGAAAGAAGAGAAGCA-3′ (SEQ ID NO: 66). The expected CRISPR/Cas9 expression vectors were purified and transformed into Agrobacterium tumefaciens strain EHA101 via electroporation, and then incubated at 28° C. on LB plates with 50 mg/L kanamycin, 50 mg/L chloromycetin, 50 mg/L spectinomycin, and 50 mg/L rifampicin for 48 h.

Some mono-clones were selected from the plates and inoculated into 1 mL LB liquid medium containing antibiotics, and then incubated at 28° C. with shaking at 180 rpm for 14 h. The bacterial liquids were test by PCR with the primers Cas9JC-F: 5′-TTGGGGCTCACACCAAACTT-3′ (SEQ ID NO: 11) and Cas9JC-R (5′-CGATCGCCTTCTTTTGCTCG-3′ (SEQ ID NO: 12). The PCR cycle was as follows: 95° C. 5 min; 94° C. 30 s, 58° C. 30 s, 72° C. 1 min, 35 cycles; 72° C. 10 min. The expected band is about 910 bp. The strains can be subsequently used for soybean transformation.

### 1.2 Transformation of the Gene Editing Vector for GmCOL2a and GmCOL2b in Soybean

Smooth and plump soybean seeds were selected, and surface sterilized using chlorine for 16~20 h. After sterilization, the seeds were cultivated in the germination culture medium containing 3.1 g/L Gamborg Basal Salt Mixture, 20 g/L sucrose, pH 5.8, and 7 g/L agar at 28° C. for one day.

Agrobacterium tumefaciens strain EHA101 containing the expected CRISPR/Cas9 vector for GmCOL2a was activated twice. Initially, the bacteria was painted on the surface of LB plates containing the appropriate antibiotics and incubate at 28° C. for 48 h, then daubed onto new solidified LB medium plates with the same antibiotics and incubated overnight at 28° C. The fresh Agrobacterium was collected by a spreader and resuspended in the liquid co-cultivation medium (2.165 g/L Murashige & Skoog Basal Salt Mixture, 30 g/L sucrose, 3.9 g/L 2-(N-Morpholino) ethanesulfonic acid (MES), 1 mL/L Gamborg vitamin solution, 2 mg/L trans-zeatin, 150 mg/L Dithiothreitol (DTT), 40 mg/L Acetosyringone (As), pH 5.4) until the OD₆₀₀ was 0.6-0.8.

Explants were prepared from 1-day-old seedlings. The cotyledon nodes were scratched and immersed in the Agrobacterium tumefaciens at 28° C. for 2 h. After inoculation, the cotyledons were placed in solid co-culture medium (2.165 g/L Murashige & Skoog Basal Salt Mixture, 30 g/L sucrose, 3.9 g/L MES, 7 g/L agar, 1 mL/L Gamborg vitamin solution, 2 mg/L trans-zeatin, 150 mg/L DTT, 40 mg/L As, pH 5.4) with a piece of Whatman filter paper and then incubated at 22° C. in the dark condition for 5 days.

After co-cultivation, the explants were transferred to recovery medium (3.1 g/L Gamborg basal salt mixture, 0.98 g/L MES, 30 g/L sucrose, 7 g/L agar, 1 mL/L Gamborg vitamin solution, 150 mg/L cefotaxime, 450 mg/L timentin, 1 mg/L 6-benzylaminopurine, 12 mg/L ferrous sulfate, 30 mg/L ethylenediaminetetraacetic acid disodium salt, 50 mg/L L-glutamine, 50 mg/L L-asparagine, pH 5.7), and incubated at 28° C. for 7 days.

After recovery, the explants were transferred to selection culture medium (3.1 g/L Gamborg basal salt mixture, 0.98 g/L MES, 30 g/L sucrose, 7 g/L agar, 1 mL/L Gamborg vitamin solution, 150 mg/L cefotaxime, 450 mg/L Timentin, 1 mg/L 6-benzylaminopurine, 12 mg/L ferrous sulfate, 30 mg/L ethylenediaminetetraacetic acid disodium salt, 50 mg/L L-glutamine, 50 mg/L L-asparagine, 6 mg/L glufosinate, pH 5.7) and incubated at 28° C. for 21 days.

After selection, the cotyledons and brown leaves were cut from the explants, and the remaining tissues were transferred to shoot elongation medium (4.0 g/L Murashige & Skoog Basal Salt Mixture, 0.6 g/L MES, 30 g/L sucrose, 7 g/L agar, 1 mL/L Gamborg vitamin solution, 150 mg/L cefotaxime, 450 mg/L timentin, 0.1 mg/L indole-3-acetic acid (IAA), 0.5 mg/L gibberellin (GA), 1 mg/L trans-zeatin, 12 mg/L ferrous sulfate, 30 mg/L ethylenediaminetetraacetic acid disodium salt, 50 mg/L L-glutamine, 50 mg/L L-asparagine, and 6 mg/L glufosinate, pH 5.7), and incubated at 28° C. until the elongated shoots grew up to 5-8 cm. Meanwhile, the culture medium was replaced every 2 weeks.

The elongated shoots were cut from the base of the buds, and the stems were dipped in 1 mg/L indole-3-butyric acid (IBA) for 1 min and then placed in a rooting culture medium (2.165 g/L Murashige & Skoog Basal Salt Mixture, 0.6 g/L MES, 20 g/L sucrose, 7 g/L agar, 1 mL/L Gamborg vitamin solution, 50 mg/L L-glutamine, 50 mg/L L-asparagine, 3 mg/L glufosinate, pH 5.7), and incubated at 28° C. for 7 days. After root production, the plants were transferred to pots and grown in the greenhouse.

1.3 Screening for Mutations of GmCOL2a after Gene Editing

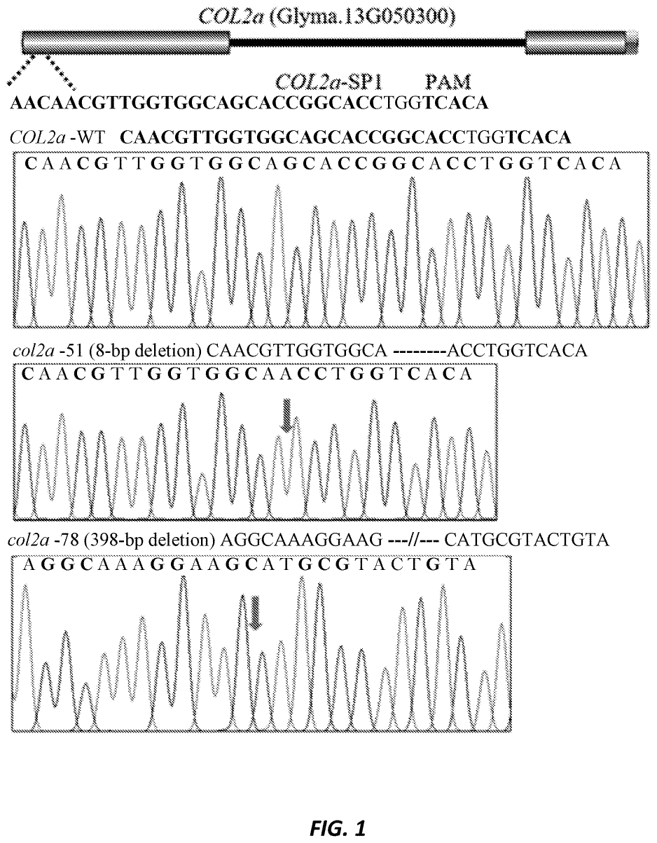

Genomic DNA was extracted from the leaves of each individual plant in the TO generation, and then the regions spanning the target sites were amplified by PCR using Phanta© Super Fidelity DNA Polymerase (Vazyme Biotech) with the GmCOL2a forward primer: 5′-AGGGATAACATGAGATTTTGACTGG-3′ (SEQ ID NO: 4) and reverse primer: 5′-CAGAGATCGGGAGAATGGGC-3′ (SEQ ID NO: 5), purified using Zymoclean™ Gel DNA Recovery Kit and sequenced by TSINGKE (Beijing). Different types of gene editing can be identified via sequence peaks. Short base insertions or deletions (not multiples of three) induced by the gene editing mechanism can lead to frameshift mutations. The heterozygous mutations showed overlapping peaks from the target sites to the end. The wild-type and homozygous mutations had no overlapping peaks at the target sites. Then, the homozygous mutant types were identified by sequence alignment with the wild-type sequence. This method was also used in the T1 and T2 generations. In this study, we detected two types of homozygous mutations at the target site of GmCOL2a in the T1 generation (FIG. 1). One type of mutation is an 8-bp deletion (from nucleotide position 535 to nucleotide position 542 of the SEQ ID NO: 15), which also results in an early flowering phenotype. The other type of mutation is 398-bp deletion (nucleotide position 182 to nucleotide position 579 of the SEQ ID NO: 15).

### 1.4 Phenotypes of the GmCOL2a Mutants

To verify whether GmCOL2a is involved in the regulation of photoperiodic flowering, the wild type (WT) plants and GmCOL2a mutants (398-bp deletion) were grown under long-day (LD, 16 h light/8 h dark in a 24-hour period) and short-day (SD, 12 h light/12 h dark in a 24-hour period) photoperiodic conditions. The flowering time of each soybean plant was recorded as days from emergence to the R1 stage (the first flower appears at any node in the main stem). For quantitative analyses of flowering time, at least 12 individual soybean plants were analyzed per genotype. Statistical analyses were performed using Microsoft Excel. A one-way analysis of variance least significant difference test (LSD) was used to compare the significance of differences between controls and treatments at the 0.01 probability level. Graphpad prism was used for drawing histograms. The flowering time is shown as the mean values±standard deviation. Under SD conditions, the flowering time of GmCOL2a mutants were almost the same as WT plants (22.31±0.48 DAE for GmCOL2a mutants vs. 22.33±0.65 DAE for WT) (Table 8 and FIG. 2). By contrast, under LD conditions and compared to WT plants, GmCOL2a mutants showed earlier flowering time by about 5 days (34.53±0.52 DAE for GmCOL2a mutants vs. 39.70±1.80 DAE for WT) (Table 8 and FIG. 2). These results indicated that CRISPR/Cas9-mediated targeted mutagenesis of GmCOL2a accelerates flowering in soybean under LD conditions.

TABLE 8
Flowering time of WT plants and GmCOL2a mutants under SD and LD
conditions.

| Plant type | R1 stage (SD) | R1 stage (LD) |
|---|---|---|
| WT | 22.33 ± 0.65 DAE | 39.70 ± 1.80 DAE |
| GmCOL2a mutants | 22.31 ± 0.48 DAE | 34.53 ± 0.52 DAE |

2.1 Screening for Mutations of GmCOL2b after Gene Editing

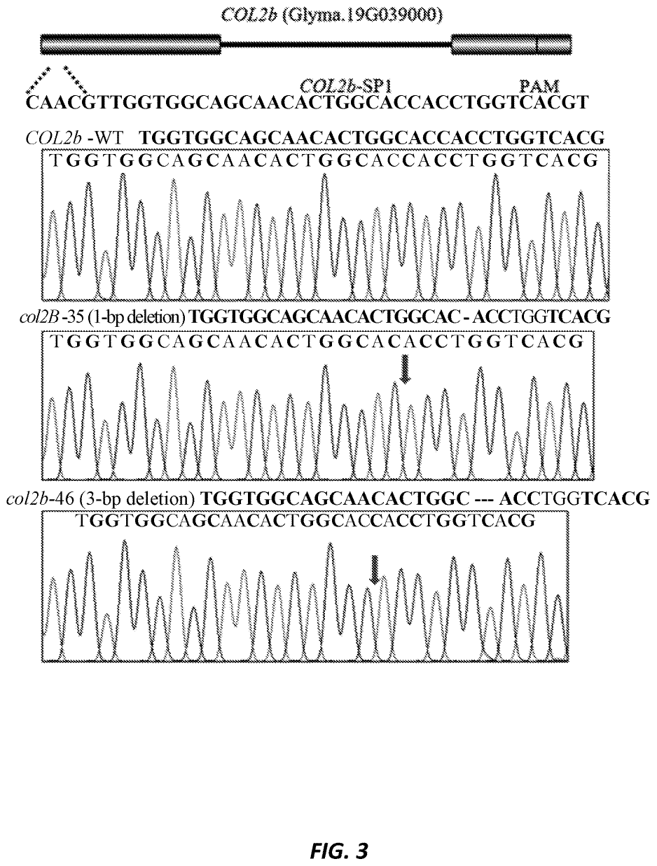

Genomic DNA was extracted from the leaves of each individual plant in the TO generation, and then the regions spanning the target sites were amplified by PCR using Phanta® Super Fidelity DNA Polymerase (Vazyme Biotech) with the GmCOL2b forward primer: 5′-ACACGTGTCTCCAAGTTGTGT-3′ (SEQ ID NO: 9) and reverse primer (5′-ACGCGTTTGTGATTGTGCTC-3′ (SEQ ID NO: 10), purified using Zymoclean™ Gel DNA Recovery Kit and sequenced by TSINGKE (Beijing). Different types of gene editing can be identified via sequence peaks. Short base insertions or deletions (not multiples of three) induced by CRISPR/Cas9 can lead to frameshift mutations. The heterozygous mutations showed overlapping peaks from the target sites to the end. The wild-type and homozygous mutations had no overlapping peaks at the target sites. Then, the homozygous mutant types were identified by sequence alignment with the wild-type sequence. This method was also used in the T1 and T2 generations. In this study, we detected two types of homozygous mutations at the target site of GmCOL2b in the T1 generation (FIG. 3). One type of the mutations is 3-bp deletion (546 to 548 bp of the SEQ ID NO: 21). The other type of the mutations is 1-bp deletion (548^{th} of the SEQ ID NO: 21). The mutants with 1-bp deletion were used in the follow-up experiments.

### 2.2 Phenotypes of the GmCol2b Mutants

The GmCOL2b mutants disclosed in this example are homozygous recessive for the respective mutant alleles, i.e., the 3-bp deletion or the 1-bp deletion as described above. To verify whether GmCOL2b is involved in the regulation of photoperiodic flowering, the wild type (WT) plants and Gmcol2b mutants (1-bp deletion) were grown under long-day (LD, 16 h light/8 h dark in a 24-hour period) and short-day (SD, 12 h light/12 h dark in a 24-hour period) photoperiodic conditions. The flowering time of each soybean plant was recorded as days from emergence to the R1 stage (the first flower appears at any node in the main stem). For quantitative analyses of flowering time, at least 12 individual soybean plants were analysed per genotype. Statistical analyses were performed using Microsoft Excel. A one-way analysis of variance, the least significant difference test (LSD), was used to compare the significance of differences between controls and treatments at the 0.01 probability level. Graphpad prism was used for drawing histograms. The flowering time is shown as the mean values±standard deviation. Under SD conditions, the flowering time of Gmcol2b mutants were almost the same as WT plants (21.93±0.27 DAE for Gmcol2b mutants vs. 22.33±0.65 DAE for WT) (Table 3 and FIG. 4). By contrast, under LD conditions and compared to WT plants, Gmcol2b mutants showed earlier flowering time by about 7 days (32.69±0.85 DAE for col2b mutants vs. 39.70±1.80 DAE for WT) (Table 9 and FIG. 4). These results indicated that CRISPR/Cas9-mediated targeted mutagenesis of GmCOL2b accelerates flowering in soybean under LD conditions.

TABLE 9
Flowering time of WT plants and GmCOL2b mutants under SD and LD
conditions.

| Plant type | R1 stage (SD) | R1 stage (LD) |
|---|---|---|
| WT | 22.33 ± 0.65 DAE | 39.70 ± 1.80 DAE |
| col2b mutants | 21.93 ± 0.27 DAE | 32.69 ± 0.85 DAE |

### 3.1 Generation of the GmCOL2a GmCOL2b Double Mutants

In order to generate the Gmcol2a Gmcol2b double mutants, we performed hybridization using T1 homozygous GmCOL2a mutants (a 398-bp deletion) as the male parent and Gmcol2b mutants (1-bp deletion) as the female parent. F2 generation plants were obtained by self-crossing. Genomic DNA was extracted from the leaves of each individual plant in the F2 generation, and then the regions spanning the target sites of GmCOL2a or GmCOL2b were amplified by PCR using Phanta® Super Fidelity DNA Polymerase (Vazyme Biotech) with the GmCOL2a forward primer: 5′-AGGGATAACATGAGATTTTGACTGG-3′(SEQ ID NO: 4), GmCOL2a reverse primer: 5′-CAGAGATCGGGAGAATGGGC-3′ (SEQ ID NO: 5) and GmCOL2b forward primer: 5′-ACACGTGTCTCCAAGTTGTGT-3′ (SEQ ID NO: 9), GmCOL2b reverse primer: 5′-ACGCGTTTGTGATTGTGCTC-3′ (SEQ ID NO: 10), purified using Zymoclean™ Gel DNA Recovery Kit and sequenced by TSINGKE (Beijing). The homozygous Gmcol2a Gmcol2b double mutants were used in the follow-up experiments.

### 3.2 Phenotypes of the GmCOL2a GmCOL2b Double Mutants

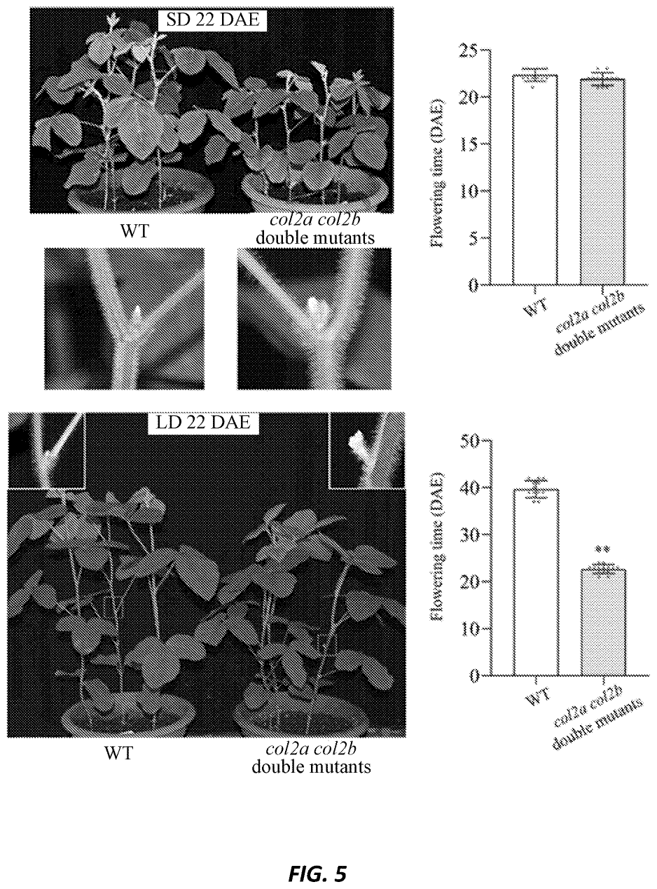

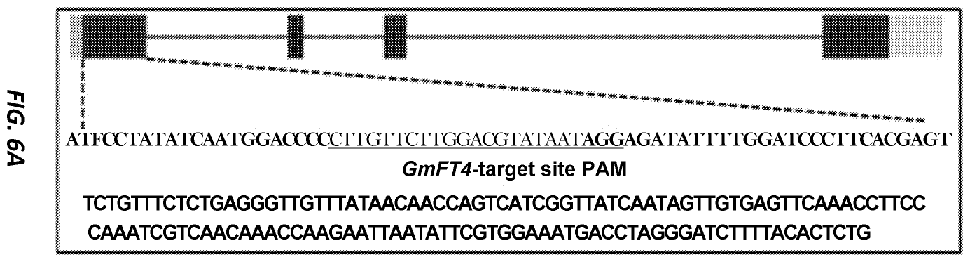

The wild type (WT) plants and Gmcol2a Gmcol2b double mutants were grown under long-day (LD, 16 h light/8 h dark) and short-day (SD, 12 h light/12 h dark) photoperiodic conditions. The flowering time of each soybean plant was recorded as days from emergence to the R1 stage (the first flower appears at any node in the main stem). For quantitative analyses of flowering time, at least 12 individual soybean plants were analysed per genotype. Statistical analyses were performed using Microsoft Excel. A one-way analysis of variance, least significant difference test (LSD), was used to compare the significance of differences between controls and treatments at the 0.01 probability level. Graphpad prism was used for drawing histograms. The flowering time is shown as the mean values±standard deviation. Under SD conditions, the flowering time of Gmcol2a Gmcol2b double mutants were almost the same as WT plants (21.92±0.67 DAE for Gmcol2a Gmcol2b double mutants vs. 22.33±0.65 DAE for WT) (Table 10 and FIG. 5). By contrast, under LD conditions and compared to WT plants, Gmcol2a Gmcol2b double mutants showed earlier flowering time about 17 days (22.69±0.95 DAE for Gmcol2a Gmcol2b double mutants vs. 39.70±1.80 DAE for WT) (Table 10 and FIG. 5). These results indicated that the Gmcol2a Gmcol2b double mutants exhibit a significantly early flowering phenotype under LD conditions.

TABLE 10
Flowering time of WT plants and GmCOL2a GmCOL2b double mutants
under SD and LD conditions.

| Plant type | R1 stage (SD) | R1 stage (LD) |
|---|---|---|
| WT | 22.33 ± 0.65 DAE | 39.70 ± 1.80 DAE |
| Gmcol2a Gmcol2b | 21.92 ± 0.67 DAE | 22.69 ± 0.95 DAE |
| double mutants | | |

### Example 2. Targeted Mutagenesis of GmFT4 Accelerates Flowering in Soybean

### 1: Generation of Gmft4 Mutant Plants

### 1.1 SgRNA Design and Construction of the Gene Editing Vector

(1) The genomic sequence of soybean GmFT4 (SEQ ID NO: 27) was obtained from Phytozome database. GnFT4 is located on chromosome 8. Using CRISPR-P (cbi.hzau.edu.cn/cgi-bin/CRISPR) The online web tool was used to select the target site sequence of GnFT4 sgRNA. The target is located in the first exon region of GmFT4, and the target sequence is 5′-CTTGTTCTTGGACGTATAATAGG-3′ (SEQ ID NO: 43) (SEQ ID NO: 27, position 64-86).

The target primers of sgRNA were synthesized and integrated into the CRISPR/Cas9 vector (ViewSolid Biotech, VK005-15, Beijing), and the primer sequences were: GmFT4-F: 5′-TTGCTTGTTCTTGGACGTATAAT-3′ (SEQ ID NO: 44) and GnFT4-R: 5′-AACATTATACGTCCAAGAACAAG-3′ (SEQ ID NO: 45).

This pair of DNA oligos were then annealed (Each 5 μL of the 10 M GmFT4-Cas9-F/R and 15 μL ddH₂O were mixed well. The mixture was then placed at 95° C. for 3 min. After that, the temperature is slowly cooled down to 16° C. by −1° C./20 s) to generate a dimer, which was subsequently integrated into the Cas9/gRNA vector (ViewSolid Biotech, VK005-15, Beijing), which contains Cas9 protein expression unit, to obtain the recombinant vector Cas9 sgRNA.

The recombinant vector Cas9 sgRNA prepared was transferred into E. coli DH5a and then incubated at 37° C. overnight on LB plates with 50 mg/L kanamycin. The monoclonal antibodies were extracted and sequenced.

Using the sequencing primer SQ: TGAAGTGGACGGAAGGAGGAGGAGG (SEQ ID NO: 67), the plasmid inserted with the correct fragment was identified and named as CRISPR/Cas9-GmFT4.

The recombinant plasmid CRISPR/Cas9-GmFT4 was transformed into Agrobacterium tumefaciens EHA105 by electroporation. The plasmid was extracted and sequenced, and the correct recombinant strain named CRISPR/Cas9-GmFT4 was verified by sequencing. The PCR reaction system was as follows: 2×Taq Master Mix, 12.5 μL; bacterial fluid, 1 μL; Cas9-F (10 μmol/μL), 1 μL; Cas9-R (10 μmol/μL), 1 μL; ddH₂O 9.5 μL, total volume 25 μL. The PCR reaction was set as follows: 94° C. for 3 min; 94° C. for 30 s, 55° C. for 30 s, and 72° C. for 30 s, for 35 cycles; and a final extension at 72° C. for 10 min. The expected band is about 910 bp. The strains can be subsequently used for soybean transformation.

### 1.2 Transformation of the Expected Gene Editing Vector for GmFT4 in Soybean

Transformation of the expected gene editing vector for GmFT4 in soybean was performed essentially as described in Example 1, section 1.2.

### 1.3 Screening for Mutations of GmFT4 Induced by the CRISPR/Cas9 System

Genomic DNA was extracted from the leaves of each individual plant in the T₀ generation, and then the regions spanning the target sites were amplified by PCR using Phanta® Super Fidelity DNA Polymerase (Vazyme Biotech) with the GmFT4 forward primer: 5′-TCACACGCGCAAGAACGTAT-3′ (SEQ ID NO: 68) and reverse primer: 5′-CTAGGAGCATCGGGGTTCAC-3′ (SEQ ID NO: 69), purified using Zymoclean™ Gel DNA Recovery Kit and sequenced by TSINGKE (Beijing). The 470 bp PCR products were sequenced and confirmed by alignment with the WT sequence. The PCR reaction system was as follows: 2×Taq Master Mix, 12.5 μL; DNA (200 ng/μL), 1 μL; GmFT4 forward primer (10 μmol/μL), 1 μL; GmFT4 reverse primer (10 μmol/μL), 1 μL; ddH₂O 9.5 μL, total volume 25 μL. The PCR reaction was set as follows: 94° C. for 3 min; 94° C. for 30 s, 54° C. for 30 s, and 72° C. for 1 min, for 35 cycles; and a final extension at 72° C. for 10 min. And all PCR products were sequenced by the GmFT4 forward primer: 5′-TCACACGCGCAAGAACGTAT-3′ (SEQ ID NO: 68). Different types of gene editing can be identified via sequence peaks.

Short base insertions or deletions induced by CRISPR/Cas9 will lead to frameshift mutations. The heterozygous mutations showed overlapping peaks from the target sites to the end. The wild-type and homozygous mutations had no overlapping peaks at the target sites. Then, the homozygous mutant types were identified by sequence alignment with the wild-type sequence. This method was also used in the T₁ and T2 generations.

The mutation types of GmFT4 gene in the mutant plants included two kinds of mutations: type 1 and type 2. Both mutant types lead to the premature termination of the protein translation and encode polypeptides having an amino acid sequence of SEQ ID NO: 30 and SEQ ID NO: 32, respectively.

Mutant type 1 comprises a 5-bp deletion from nucleotide position 76 to nucleotide position 80 of a polynucleotide having the nucleic acid sequence of SEQ ID NO: 27 (the other nucleotides remain unchanged). The CDS sequence of GmFT4 in Gmft4 mutants (5-bp deletion) is shown in SEQ ID NO: 57.

Mutant type 2 comprises a 1-bp insertion, a T between nucleotide position 80 and nucleotide position 81 of a polypeptide having the nucleic acid sequence SEQ ID NO: 27 (the other nucleotides remain unchanged). The CDS sequence of GmFT4 in Gmft4 mutants (1-bp insertion) is shown in SEQ ID NO: 58.

The T₁ transgenic GmFT4 soybean mutant plants with various GmFT4 gene mutation types were continued to cultivate until T2 and after harvest, the seeds of T3 transgenic GmFT4 soybean (Gmft4 homozygous mutant) were harvested.

### 1.4 Identification of Flowering and Maturity of Gmft4 Mutations

Materials: soybean Jack (WT), T3 transgenic GmFT4 mutant soybean homozygous lines Gmft4-73, Gmft4-81 and Gmft4-122.

Method: the investigation of each period is carried out according to the standard recording method of soybean growth period proposed by Fehr et al. (Fehr et al. Nov. 1, 1971, Crop Science, available at doi.org/10.2135/cropsci1971.0011183X001100060051x). The standards of each period are shown in Table 2. This experiment needs to investigate the emergence stage (VE, cotyledon unearthed) and first flowering stage (R1 stage, first flowering time, there is an open flower at any node on the main stem of soybean). The materials were planted in artificial culture rooms under the conditions of long sunshine (LD, 16 h light, 30° C./8 h dark, 22° C.) and short sunshine (SD, 12 h light, 30° C./12 h dark, 22° C.). The flowering time of each soybean plant was recorded as days from emergence (VE) to the R1 stage (the time at which the first flower appears at any node on the main stem), and the maturity time was recorded from VE to R7 (the time at which the first pod has reaches the maturity color on the main stem) by Fehr & Caviness, 1977.

Under SD conditions, The R1 of the two Gmft4 mutants were 23.7±2.0 d (type 1) and 23.3±1.1 d (type 2), respectively, which had no significant difference compared with that of WT (23.7:±2.1 d) (p>0.05, Table 11); the R7 of the two Gmft4 mutants were 64.9:±2.2 d and 62.0±1.7 d, respectively, and 2.9~5.8 d earlier than that of WT (67.8±2.5 d) (p<0.05, Table 10), indicating that the GmFT4 did not affected the flowering date but accelerated the post-flowering growth under SD conditions. There was no significant difference in plant height and node number between wild type and mutant (p>0.05); while comparing with the seed number of WT (33.6±5.3), the two mutant types were 25.9±2.9 (p<0.05) and 32.1±2.1 (p>0.05), indicating type 1 might affect the seed number but the type 2 maintained the similar yield potential with WT.

Under LD conditions, the R1 of the two Gmft4 mutants were 40.6±1.5 d (type 1) and 40.6±2.1 d (type 2), respectively, and about 3 days earlier compared with that of WT (44.1±2.8 d) (p<0.05, Table 12); the R7 of the two Gmft4 mutants were 132.7±3.8 d and 136.0±3.5 d, respectively, and 5.0-8.3 d earlier than that of WT (141.0±3.5 d) (p<0.05, Table 12), indicating that the GmFT4 promoted both flowering and post-flowering maturity under LD conditions. There was no significant difference in plant height and node number between wild type and mutant (p>0.05); the pod and seed number were slightly reduced comparing with those of the WT (p>0.05), indicating the mutant of GmFT4 accelerated maturity without significant reduction of pod or seed number.

TABLE 11
Phenotypic statistics of Gmft4 mutant under SD conditions

| | | | | Plant | | | |
|---|---|---|---|---|---|---|---|
| | Plants | R1 | R7 | height | Node | Pod | Seed |
| Materials | number | (d) | (d) | (cm) | number | number | number |
| WT | 18 | 23.7 ± 2.1 | 67.8 ± 2.5 | 70.3 ± 3.8 | 7.4 ± 0.8 | 17.3 ± 2.2 | 33.6 ± 5.3 |
| Gmft4 Type 1 | 15 | 23.7 ± 2.0 | 64.9 ± 2.2* | 67.7 ± 3.4 | 6.9 ± 0.4 | 16.3 ± 1.3 | 25.9 ± 2.9* |
| Gmft4 Type 2 | 15 | 23.3 ± 1.1 | 62.0 ± 1.7* | 70.4 ± 4.7 | 7.6 ± 0.7 | 18.8 ± 0.9 | 32.1 ± 2.1 |

Note:
SD: 12h light/12 h dark

TABLE 12
Phenotypic statistics of Gmft4 mutant under LD conditions

| | | | | Plant | | | |
|---|---|---|---|---|---|---|---|
| | Plants | R1 | R7 | height | Node | Pod | Seed |
| Materials | number | (d) | (d) | (cm) | number | number | number |
| WT | 18 | 44.1 ± 2.8 | 141.0 ± 3.5 | 175.7 ± 22.5 | 19.5 ± 2.5 | 40.5 ± 3.5 | 81.7 ± 5.7 |
| Gmft4 Type 1 | 15 | 40.6 ± 1.5* | 132.7 ± 3.8* | 170.4 ± 20.4 | 19.0 ± 2.1 | 38.7 ± 4.2 | 75.5 ± 4.1 |
| Gmft4 Type 2 | 15 | 40.6 ± 2.1* | 136.0 ± 3.5* | 171.2 ± 27.1 | 19.1 ± 2.0 | 36.2 ± 2.7 | 76.3 ± 3.3 |

Note:
LD: 16 h light/8 h dark in a 24-hour period

### Example 3. Targeted Mutagenesis of GmFT5a and GmFT5b Modifies Flowering Time in Soybean

### 1. Generation of Gmft5b Mutant Plants

### 1.1 SgRNA Design and Construction of the Gene Editing Vector

The CRISPR/Cas9 vector (VK005-15, ViewSolid Biotech, Beijing) was used for sgRNA construction and expression. The sequence of Cas9 was codon-optimized for dicotyledons and assembled downstream of the CaMV 2×35S promoter together with a customized sgRNA driven by the Arabidopsis U6 promoter (SEQ ID NO: 34). The bar gene driven by a CaMV 35S promoter was used as a screening marker.

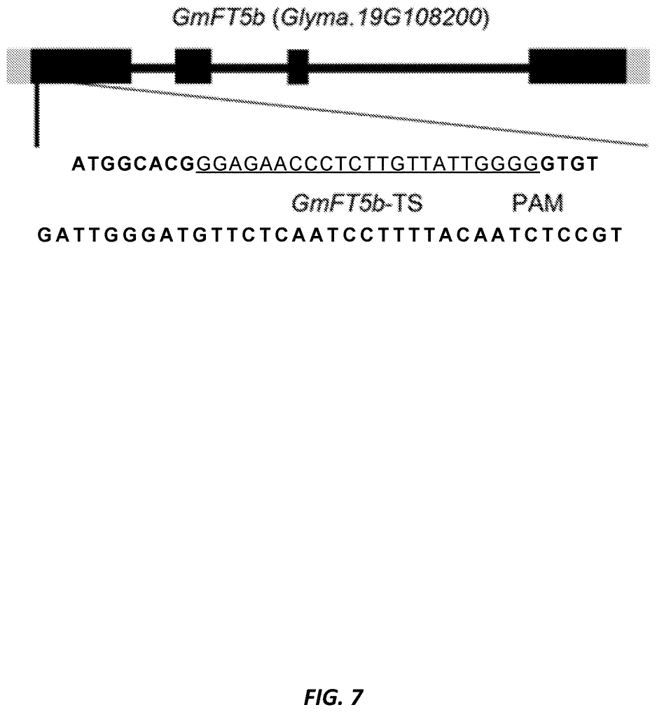

The genomic sequence of GnFT5b was obtained from Phytozome database according to the Glyma.19G108200 gene, which was located on chromosome 19. The target site of GmFT5b (GmFT5b-TS) was designed by CRISPR-P software (cbi.hzau.edu.cn/cgi-bin/CRISPR). The sequence of GmFT5b-TS was 5′-GGAGAACCCTCTTGTTATTGGGG-3′ (SEQ ID NO: 46), which was located on the first exon (from 34 to 56 of the SEQ ID NO: 54) (FIG. 7).

To get the CRISPR/Cas9-GmFT5b vector, the primers of GmFT5b-sense: 5′-TTGGGAGAACCCTCTTGTTATTG-3′ (SEQ ID NO: 47); GmFT5b-anti: 5′-AACCAATAA CAAGAGGGTTCTCC-3′ (SEQ ID NO: 48) were synthesized from TSINGKE (Beijing).

To generate GmFT5b dimer, the reaction system was as follows: GmFT5b-sense, 5 μL; GmFT5b-antisense, 5 μL; ddH₂O 15 μL; total volume 25 μL; 95° C. for 3 min and natural cooling to 25° C. The dimer was then integrated into the CRISPR/Cas9 vector.

The GmFT5b dimers chains were subcloned into the CRISPR/Cas9 vector with the help of T4 DNA ligase. The reaction system was as follows: CRISPR/Cas9 vector, 1 μL; GmFT5b dimer, 1 μL; Solution 1, 1 μL; Solution 2, 1 μL; ddH₂O 6 μL; total volume 10 μL; 16° C. for 2 h.

The ligation product was transformed into E. coli DH5a competent cells, then incubated on ice for 30 min, heated shock at 42° C. for 90 s in a water bath, and then incubated on ice for 2 min, added 700 μL LB liquid medium (10 g/L tryptone, 5 g/L yeast extract and 10 g/L NaCl), and incubated at 37° C. with shaking at 180 rpm for 1 h. We then spread all bacteria on LB plates (10 g/L tryptone, 5 g/L yeast extract, 10 g/L NaCl, and 15 g/L agar) with 50 mg/L kanamycin and incubated them overnight at 37° C.

The recombinant vector was named as CRISPR/Cas9-GmFT5b. Mono-clones were confirmed by sequencing (TSINGKE, Beijing) using the primer sqprimer: 5′-GATGAAGTGGACGGAAGGAAGGAG-3′(SEQ ID NO: 70). The consequent construct was purified using the TIANprep Rapid Mini Plasmid Kit (TIANGEN, DP103-200) for subsequent use.

The CRISPR/Cas9-GmFT5b plasmid was transformed into Agrobacterium tumefaciens EHA105 strain via electroporation, and then incubated at 28° C. on LB plates with 50 mg/L kanamycin and 50 mg/L rifampicin for 48 h. And the EHA105 mono-clones were verified by PCR with the primers (Cas9JC-F 5′-TTGGGGCTCACACCAAACTT-3′ (SEQ ID NO: 11); Cas9JC-R 5′-CGATCGCCTTCTTTTGCTCG-3′ (SEQ ID NO: 12) and sequencing. The PCR reaction system was as follows: 2×Taq Master Mix, 12.5 μL; bacterial fluid, 1 μL; Cas9-F (10 μmol/μL), 1 μL; Cas9-R (10 μmol/μL), 1 μL; ddH₂O 9.5 μL, total volume 25 μL. The PCR reaction was set as follows: 94° C. for 3 min; 94° C. for 30 s, 55° C. for 30 s, and 72° C. for 1 min, for 35 cycles; and a final extension at 72° C. for 10 min. The expected band is about 910 bp. The EHA105 mono-clones carrying the CRISPR/Cas9-GmFT5b plasmid were used for soybean transformation.

### 1.2 Transformation of CRISPR/Cas9-GmFT5b in Soybean

1.2.1. Plant materials

The transformation of soybean was followed as described by Chen, L. et al. (2018) Improvement of soybean Agrobacterium-mediated transformation efficiency by adding glutamine and asparagine into the culture media, International Journal of Molecular Sciences, 19 (10):3039. The soybean variety Jack was utilized for Agrobacterium-mediated transformation. Healthy seeds were surface-sterilized by exposure to chlorine gas for 16 h. Sterilized seeds were placed in germination culture medium (GCM) containing 3.1 g/L Gamborgs Basal Salt Mixture (Phytotech, G768, Lenexa, KS, USA), 20 g/L sugar, 1 mL/L Gamborgs Vitamin Solution (Phytotech, G219, Lenexa, KS, USA) and 7 g/L agar (Sigma, St. Louis, MO, USA), pH 5.8, and the seeds were germinated at 25° C. for 18~20 h on the light.

### 1.2.2. Agrobacterium Strain and Vector

A. tumefaciens strain EHA105 was used in the experiments. The CRISPR/Cas9 vector (ViewSolid Biotech, VK005-15, Beijing) carried T-DNA and the bar gene acted as an herbicide resistance marker.

### 1.2.3. Agrobacterium Preparation

Agrobacterium strain stocks of EHA105 stored at −80° C. were streaked on solidified YEP medium plates containing 5 g/L NaCl, 10 g/L tryptone, 5 g/L yeast extract, and 15 g/L agar, with 50 mg/L kanamycin and 50 mg/L rifampicin. Plates streaked with Agrobacterium were incubated at 28° C. for approximately 2 days until colony formation. The colonies were collected by a spreader, daubed onto new solidified YEP medium plates with the same antibiotics and incubated overnight at 28° C. The fresh Agrobacterium were resuspended in liquid co-cultivation medium (LCCM) containing ½ Murashige & Skoog Basal Salt Mixture (Phytotech, M524, Lenexa, KS, USA), 3.9 g/L MES, 30 g/L sucrose, 1 mL/L Gamborgs Vitamin Solution, 150 mg/L DTT, 2 mg/L zeatin and 40 mg/L As, pH 5.4. The OD₆₀₀ of the Agrobacterium strain was 0.6-0.8.

### 1.2.4. Infection and Co-Cultivation

Explants were prepared from one-day-old seedlings. A longitudinal cut along the hilum was made to separate the cotyledons, and the seed coat was removed. The embryonic axis found at the junctions of the hypocotyls and the cotyledon was excised to obtain the half-seed explants. The explant cuttings were immersed in Agrobacterium for 2 h at 50 rpm. After inoculation, each of the 9 cotyledons were placed in solid co-culture medium (CCM) containing ½ Murashige & Skoog Basal Salt Mixture, 3.9 g/L MES, 30 g/L sucrose, 1 mL/L Gamborgs Vitamin Solution, 150 mg/L DTT, 40 mg/L As, 2 mg/L zeatin 7 g/L agar, pH 5.4, with a piece of Whatman filter paper and then incubated at 22° C. in the dark for 5 days.

### 1.2.5. Recovery Culture and Selection Culture

After co-cultivation, explants were then transferred to recovery medium (SIMO) containing 3.1 g/L Gamborgs Basal Salt Mixture, 0.98 g/L MES, 30 g/L sucrose, 1 mL/L Gamborgs Vitamin Solution, 150 mg/L cefotaxime, 450 mg/L timentin, 1 mg/L 6-Benzylaminopurine (6-BA), and 7 g/L agar, pH 5.7, and incubated at 28° C. for 7 days. Seven days after recovery, the explants were transferred to selection culture medium (SIM6) containing 3.1 g/L Gamborgs Basal Salt Mixture, 0.98 g/L MES, 30 g/L sucrose, 1 mL/L Gamborgs Vitamin Solution, 150 mg/L cefotaxime, 450 mg/L timentin, 1 mg/L 6-BA, 7 g/L agar, and 6 mg/L glufosinate, pH 5.7, and incubated at 28° C. for 21 days.

### 1.2.6. Shoot Elongation and Rooting

After selection culture, the cotyledons and brown leaves were cut from the explants, and the remaining tissues were transferred to shoot elongation medium (SEM) containing 4.0 g/L Murashige & Skoog Basal Salt Mixture, 0.6 g/L MES, 30 g/L sucrose, 1 mL/L Gamborgs Vitamin Solution, 150 mg/L cefotaxime, 450 mg/L timentin, 0.1 mg/L IAA, 0.5 mg/L GA, 1 mg/L zeatin, 7 g/L agar, and 6 mg/L glufosinate, pH 5.6, and incubated at 28° C. The culture medium was changed every two weeks. Simultaneous with changing the SEM, the elongated shoots (5-8 cm) were cut from the base of the buds, and the stems were dipped in 1 mg/L IBA for 1 min, placed in a rooting culture medium (RCM) containing ½ Murashige & Skoog Basal Salt Mixture, 0.6 g/L MES, 20 g/L sucrose, 1 mL/L Gamborgs Vitamin Solution, and 7 g/L agar, 3 mg/L glufosinate, pH 5.7, and incubated at 28° C. for 7 days. After root production, the plants were transferred to pots and grown in the greenhouse.

### 1.3 Screening for GmFT5b Mutant Plants by Sequencing Analysis

We then screened 33 independent T₀ transgenic Gmft5b mutant lines by PCR and Sanger sequencing. To get Gmft5b mutant plants, the GmFT5b-661-F/R primers (GmFT5b-661-F: 5′-TTGACCATGCACCAAGGGAA-3′ (SEQ ID NO: 71); GmFT5b-661-R: 5′-CAAGACAG GGTTGCTAGGGC-3′ (SEQ ID NO: 72) were used to amplify the GmFT5b fragment. The 661 bp PCR products were sequenced and confirmed by alignment with the WT sequence. The PCR reaction system was as follows: 2×Taq Master Mix, 12.5 μL; DNA (200 ng/μL), 1 μL; GmFT5b-661-F (10 μmol/μL), 1 μL; GmFT5b-661-R (10 μmol/μL), 1 μL; ddH₂O 9.5 μL, total volume 25 μL. The PCR reaction was set as follows: 94° C. for 3 min; 94° C. for 30 s, 54° C. for 30 s, and 72° C. for 1 min, for 35 cycles; and a final extension at 72° C. for 10 min. And all PCR products were sequenced by GmFT5b-661-R: 5′-CAAGACAG GGTTGCTAGGGC-3′ (SEQ ID NO: 72). Finally, one homozygous “transgene-free” Gmft5b mutant was obtained carrying a frameshift mutation in T1 generation. The Gmft5b mutant harbored an 8-bp deletion (SEQ ID NO: 55 and SEQ ID NO: 37) that resulted in a frameshift-induced premature stop codon in GmFT5b (SEQ ID NO: 38). The progeny of the homozygous Gmft5b mutant were all “transgene-free” homozygous Gmft5b mutants, confirmed by libertylink strips.

### 1.4. Soybean Growth Conditions

The wild-type (WT) and Gmft5b mutant plants were grown and evaluated under short-day (SD; 12 h light and 12 h dark, 22~30° C.), long-day (LD; 16 h light and 8 h dark, 22~30° C.) in March 2021. The red-to-blue quantum (R:B) ratio of the light was 5.17. The detailed information of the light used was:

| Model | Value | |
|---|---|---|
| Lux | 11068 | 1x |
| CCT | 3190 | K |
| PPFD | 299.72 | μmol/m²s |
| PPF-UV | 0.40 | μmol/m²s |
| PPF-B | 38.42 | μmol/m²s |
| PPF-G | 62.21 | μmol/m²s |
| PPF-R | 198.75 | μmol/m²s |
| PPF-NIR | 48.68 | μmol/m²s |

### 1.5 Phenotyping and Statistical Analysis

The flowering time was evaluated as described by Fehr et al. (1971). Briefly, the flowering time of each soybean plant was recorded as days from emergence to the R1 stage (one flower at any node), and the physiological maturity was recorded as days from emergence to the R7 stage (any pod becomes to the maturity). The plant height was measured from cotyledon node to stem tip. The cotyledon node was counted as the first node. We also counted the number of pods and seeds per plant. Statistical analyses were performed using Microsoft Excel. The significant differences determined by one-way ANOVA. These data are shown as the mean values one standard deviation.

### 1.6 the Phenotypes of the Gmft5b Mutant Plants Under Different Photoperiod Conditions

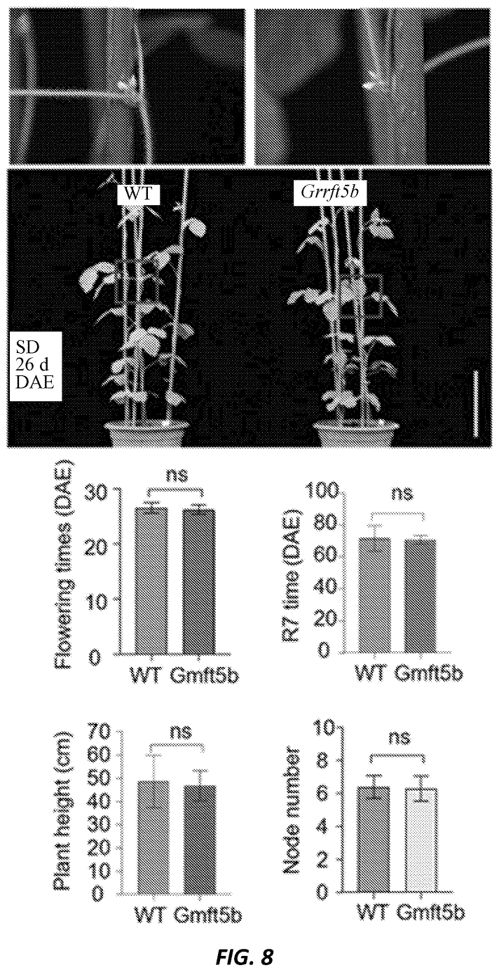

Under SD conditions, the average flowering time of the Gmft5b mutant plants, 26.3±0.84 DAE, was not significantly different from that of WT, 26.6±0.96 DAE (FIG. 8). Besides, the average R7 (beginning maturity) time of Gmft5b mutant plants was 70.6±2.7 DAE, which was not significantly different from that of WT (71.7±8.0 DAE). In addition, the average plant height of Gmft5b mutant plants (46.7±6.5 cm) was not significantly different from WT plants (48.5±11.3 cm). The average node number of Gmft5b mutant plants (6.3±0.76) was not significantly different from WT plants (6.4±0.7) (FIG. 8, Table 13).

TABLE 13
The phenotypes of WT and Gmft5b plants under SD conditions.

| Plants | R1(Flowering)(d) | R7(Maturity)(d) | Plant height(cm) | Node number |
|---|---|---|---|---|
| Gmft5b | 26.3 ± 0.84 | 70.6 ± 2.7 | 46.7 ± 6.5 | 6.3 ± 0.76 |
| WT | 26.6 ± 0.96 | 71.7 ± 8.0 | 48.5 ± 11.3 | 6.4 ± 0.7 |

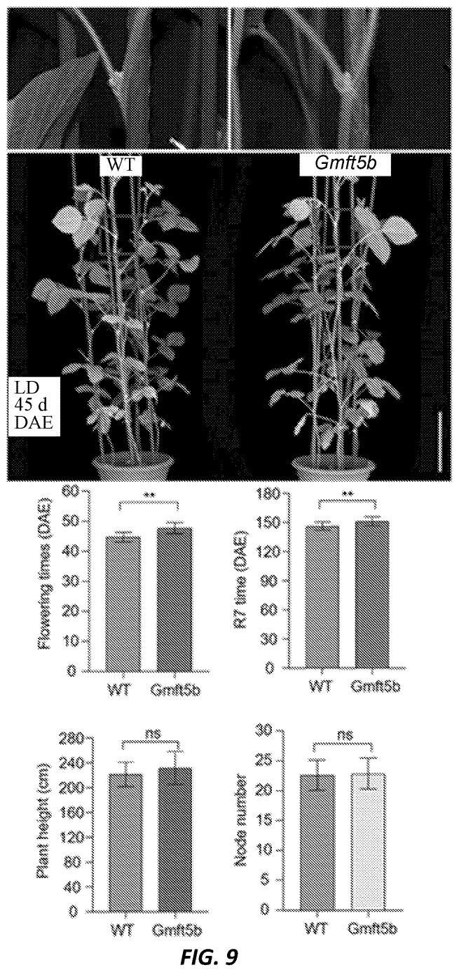

Under LD conditions, the average flowering time of Gmft5b mutants was significantly later than that of WT plants (47.8±1.8 vs 44.8±1.6 DAE, respectively) (FIG. 9). Besides, the average R7 time of Gmft5b mutant plants was 151.4±4.7 DAE, which was significantly late than that of WT (146.5±3.9 DAE). In addition, the average plant height of Gmft5b mutant plants (231.9±26.7 cm) was not significantly different from that of WT plants (221.5±19.8 cm). The average node number of Gmft5b mutant plants (23.0±2.6) was not significantly different from that of WT plants (22.6±2.5) (FIG. 9, Table 14). One way ANOVA was used for statistics. These observations strongly suggested that loss of Gmft5b function led to delayed flowering in soybean under LD, but not under SD conditions.

TABLE 14
The phenotypes of WT andGmft5b plants under LD conditions.

| Plants | R1(Flowering)(d) | R7(Maturity)(d) | Plant height(cm) | Node number |
|---|---|---|---|---|
| Gmft5b | 47.8 ± 1.8** | 151.4 ± 4.7** | 231.9 ± 26.7 | 23.0 ± 2.6 |
| WT | 44.8 ± 1.6 | 146.5 ± 3.9 | 221.5 ± 19.8 | 22.6 ± 2.5 |

**represent p < 0.01.

### 2. Generation of Gmft5a Mutant Plants

### 2.1 SgRNA Design and Construction of the CRISPR/Cas9 Expression Vector

The CRISPR/Cas9 vector (VK005-15, ViewSolid Biotech, Beijing) was used for sgRNA construction and expression. The sequence of Cas9 was codon-optimized for dicotyledons and assembled downstream of the CaMV 2×35S promoter together with a customized sgRNA driven by the Arabidopsis U6 promoter (SEQ ID NO: 34). The bar gene driven by a CaMV 35S promoter was used as a screening marker.

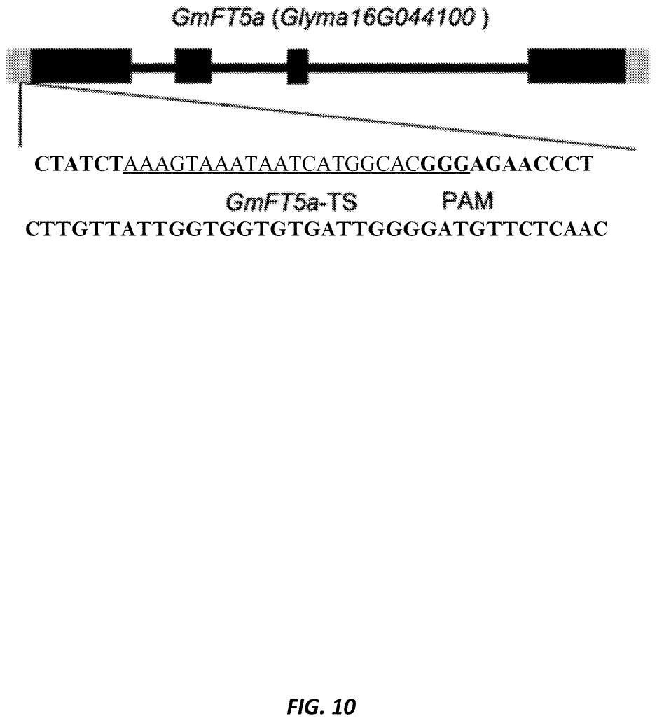

The genomic sequence of GmFT5a was obtained from Phytozome database according to the Glyma.16G044100 gene, which was located on chromosome 16. The target site of GmFT5a (GmFT5a-TS) was designed by CRISPR-P software (cbi.hzau.edu.cn/cgi-bin/CRISPR. The sequence of GmFT5a-TS was 5′-AAAGTAAATAATCATGGCACGGG-3′ (SEQ ID NO: 49), which was located on the first exon of GmFT5a (36 to 58 of the SEQ ID NO: 52) (FIG. 10).

To get the CRISPR/Cas9-GmFT5a vector, the primers of GmFT5a-TS ((GmFT5a-sense: 5′-TTGAAAGTAAATAATCATGGCAC-3′ (SEQ ID NO: 50); GmFT5a-anti: 5′-AACGTGCCAT GATTATTTACTTT-3′ (SEQ ID NO: 51) were synthesized from TSINGKE (Beijing).

To generate GmFT5a dimers chains, the reaction system was as follows: GmFT5a-sense, 5 μL; GmFT5a-anti, 5 μL; ddH₂O 15 μL; total volume 25 μL; 95° C. for 3 min and natural cooling to 25° C. The dimer was then integrated into the CRISPR/Cas9 vector.

The GmFT5a dimers chains were subcloned into the CRISPR/Cas9 vector with the help of T4 ligase. The reaction system was as follows: CRISPR/Cas9 vector, 1 μL; GmnFT5a dimers chains, 1 μL; Solution 1, 1 μL; Solution 2, 1 μL; ddH₂O 6 μL; total volume 10 μL; 16° C. for 2 h.

The ligation product was transformed into E. coli DH5a competent cells, then incubated on ice for 30 min, heated shock at 42° C. for 90 s in a metal bath or water bath, and then incubated on ice for 2 min, added 700 μL LB liquid medium (10 g/L tryptone, 5 g/L yeast extract and 10 g/L NaCl), and incubated at 37° C. with shaking at 180 rpm for 1 h. We then spread all bacteria on LB plates (10 g/L tryptone, 5 g/L yeast extract, 10 g/L NaCl, and 15 g/L agar) with 50 mg/mL kanamycin and incubated them overnight at 37° C.

The recombinant vector was named as CRISPR/Cas9-GmFT5a. Some mono-clones were confirmed by sequencing (TSINGKE, Beijing) using the primer sqprimer: 5′-GATGAAGTGGACGGAAGGAAGGAG-3′ (SEQ ID NO: 70). The consequent construct was purified using the TIANprep Rapid Mini Plasmid Kit (TIANGEN, DP103-200) for subsequent use.

The CRISPR/Cas9-GmFT5a plasmid was transformed into Agrobacterium tumefaciens EHA105 strain via electroporation, and then incubated at 28° C. on LB plates with 50 mg/L kanamycin and 50 mg/L rifampicin for 48 h. And the EHA105 mono-clones were verified by PCR with the primers (Cas9JC-F 5′-TTGGGGCTCACACCAAACTT-3′ (SEQ ID NO: 11); Cas9JC-R 5′-CGATCGCCTTCTTTTGCTCG-3′ (SEQ ID NO: 12) and sequencing. The PCR reaction system was as follows: 2×Taq Master Mix, 12.5 μL; bacterial fluid, 1 μL; Cas9-F (10 μmol/μL), 1 μL; Cas9-R (10 μmol/μL), 1 μL; ddH₂O 9.5 μL, total volume 25 μL. The PCR reaction was set as follows: 94° C. for 3 min; 94° C. for 30 s, 55° C. for 30 s, and 72° C. for 1 min, for 35 cycles; and a final extension at 72° C. for 10 min. The expected band is about 910 bp. The EHA105 mono-clones carrying the CRISPR/Cas9-GmFT5a plasmid were used for soybean transformation.

### 2.2 Transformation of CRISPR/Cas9-GmFT5a in Soybean

Transformation of CRISPR/Cas9-GmFT5a into soybean was performed using materials and methods as described Section 1.1 of this example.

### 2.3 Screening for Gmft5a Mutant Plants by Sequencing Analysis

We screened T0 transgenic Gmft5a mutant lines by PCR and Sanger sequencing. To get Gmft5a mutant plants, we used the GmFT5a-616-F/R primers (GmFT5a-616-F: 5′-ATCGACCGATCGAGGACAAC-3′ (SEQ ID NO: 73); GmFT5a-616-R: 5′-TGGGAGACTACAGAAGCAAAGA-3′ (SEQ ID NO: 74) to amplify the GmFT5a fragment. The 616 bp PCR products were sequenced and confirmed by alignment with the WT sequence. The PCR reaction system was as follows: 2×Taq Master Mix, 12.5 μL; DNA (200 ng/μL), 1 μL; GmFT5a-616-F (10 μmol/μL), 1 μL; GmFT5a-616-R (10 μmol/μL), 1 μL; ddH₂O 9.5 μL, total volume 25 μL. The PCR reaction was set as follows: 94° C. for 3 min; 94° C. for 30 s, 54° C. for 30 s, and 72° C. for 1 min, for 35 cycles; and a final extension at 72° C. for 10 min. Finally, we obtained one homozygous Gmft5a mutant carrying a frameshift mutation in T1 generation. The Gmft5a mutant harbored a 1-bp insertion (SEQ ID NO: 41 and SEQ ID NO: 53) that resulted in a frameshift-induced premature stop codon in Gmft5a (SEQ ID NO: 42). The progeny of the homozygous Gmft5a mutant were all “transgene-free” homozygous Gmft5a mutants.

### 2.4 Soybean Materials and Growth Conditions

The wild-type (WT) and Gmft5a mutant plants were grown and evaluated under short-day (SD; 12 h light and 12 h dark, 22~30° C.) and long-day (LD; 16 h light and 8 h dark, 22~30° C.) in March 2021. The red-to-blue quantum (R:B) ratio of the light was 5.17. The same light as described in Sec. 1.4 of this example above was used.

### 2.5 the Phenotypes of the Gmft5a Mutant Plants Under Different Photoperiod Conditions

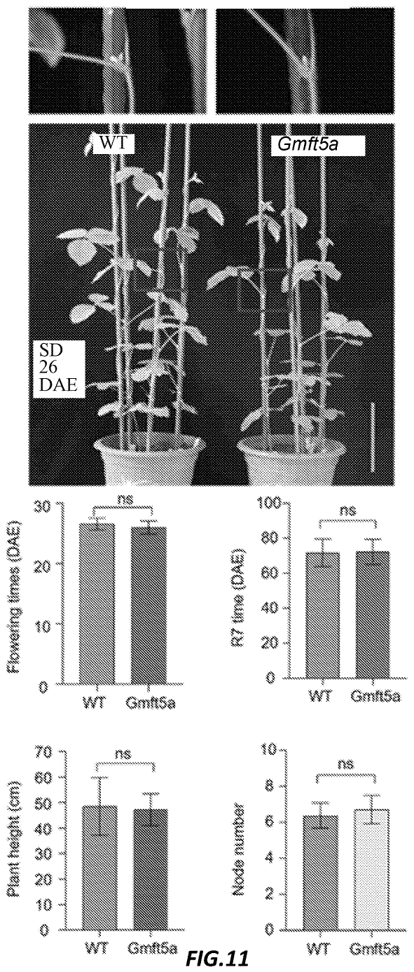

Under SD conditions, the average flowering time of the Gmft5a mutant plants (26.1±1.1 DAE) was not significantly different from that of WT (26.6±0.96 DAE). Besides, the average R7 time of Gmft5a mutant plants was 72.1±7.3 DAE, which was not significantly different from that of WT (71.7±8.0 DAE). In addition, the average plant height of Gmft5a mutant plants (47.3±16.3 cm) was not significantly different from WT plants (48.5±111.3 cm). The average node number of Gmft5a mutant plants (6.7±0.8) was not significantly different from WT plants (6.4±0.7) (FIG. 11, Table 15).

TABLE 15
The phenotypes of WT and Gmft5a plants under SD conditions.

| Plants | R1(Flowering)(d) | R7(Maturity)(d) | Plant height(cm) | Node number |
|---|---|---|---|---|
| Gmft5a | 26.1 ± 1.1 | 72.1 ± 7.3 | 47.3 ± 6.3 | 6.7 ± 0.8 |
| WT | 26.6 ± 0.96 | 71.7 ± 8.0 | 48.5 ± 11.3 | 6.4 ± 0.7 |

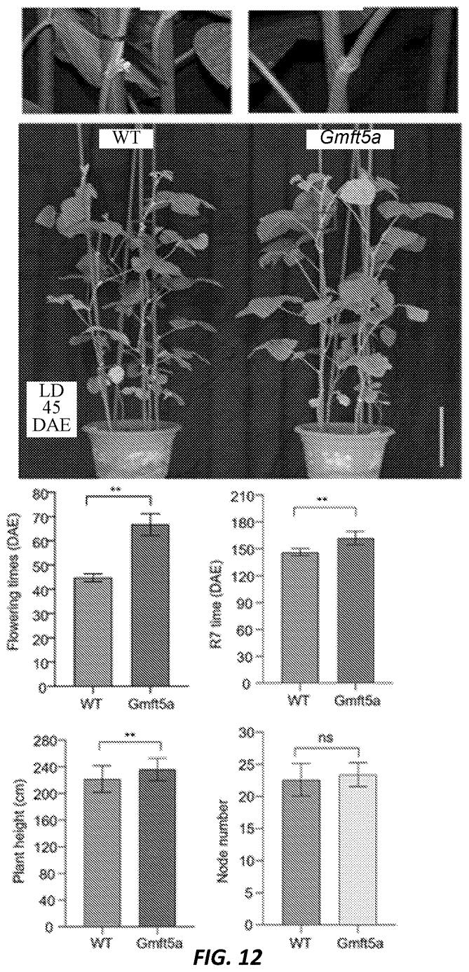

Under LD conditions, the average flowering time of Gmft5a mutants was significantly later than that of WT plants (66.6±4.6 vs 44.8±1.6 DAE, respectively) (FIG. 12). Besides, the average R7 time of Gmft5a mutant plants was 161.9±7.5 DAE, which was significantly late than that of WT (146.5±3.9 DAE). In addition, the average plant height of Gmft5a mutant plants was 236.1±16.9 cm that was not significantly different from that of WT plants (221.5±19.8 cm). The average node number of Gmft5a mutant plants (23.3±5.0) was not significantly different from that of WT plants (22.6±2.5) (FIG. 12, Table 16). One way ANOVA was used for statistics. These observations strongly suggested that loss of Gmft5a function led to delayed flowering in soybean under LD, but not under SD conditions.

TABLE 16
The phenotypes of WT and Gmft5a plants under LD conditions.

| Plants | R1(Flowering)(d) | R7(Maturity)(d) | Plant height(cm) | Node number |
|---|---|---|---|---|
| Gmft5a | 66.6 ± 4.6** | 161.9 ± 7.5** | 236.1 ± 16.9 | 23.3 ± 5.0 |
| WT | 44.8 ± 1.6 | 146.5 ± 3.9 | 221.5 ± 19.8 | 22.6 ± 2.5 |

**represent p < 0.01.

### 3. Generation of Gmft5a Gmft5b Double Mutant Plants

### 3.1 Creating the Gmft5a Gmft5b Hybrid Line

Firstly, the T3 homozygous Gmft5a and Gmft5b mutant plants were planted under natural long day conditions. We then performed hybridization using Gmft5a mutant plants (1-bp insertion) as the male parent and Gmft5b mutant plants (an 8-bp deletion) as the female parent to generate F1 generation of Gmft5a Gmft5b double mutant plants.

### 3.2 Screening for Homozygous Gmft5a Gmft5b Double Mutant Plants

Firstly, the F1 generation of Gmft5a Gmft5b double mutant plants were planted under short day conditions. And the genomic DNA was extracted with TPS buffer (100 mM Tris-HCl, 10 mM EDTA, 1 M KCl, pH 8.0) as follows. Briefly, ~100 mg soybean leaves were macerated in TPS buffer, and the supernatant was obtained by centrifugation. Genomic DNA was then extracted by anhydrous alcohol precipitation at −20° C. Subsequently, DNA from each plant had the GmFT5a and GmFT5b fragment amplified by PCR using the GmFT5a-616-F/R primers (GmFT5a-616-F: 5′-ATCGACCGATCGAGGACAAC-3′ (SEQ ID NO: 73); GnFT5a-616-R: 5′-TGGGAGACTACAGAAGCAAAGA-3′ (SEQ ID NO: 74) and GmFT5b-661-F/R primers (GmFT5b-661-F: 5′-TTGACCATGCACCAAGGGAA-3′ (SEQ ID NO: 71); GmFT5b-661-R: 5′-CAAGACAGGGTTGCTAGGGC-3′ (SEQ ID NO: 72)), respectively. The PCR reaction system was as follows: 2×Taq Master Mix, 12.5 μL; DNA (200 ng/μL), 1 μL; GmFT5b-661-F(10 μmol/μL), 1 μL; GmFT5b-661-R (10 μmol/μL), 1 μL; ddH₂O 9.5 μL, total volume 25 μL. 2×Taq Master Mix, 12.5 μL; DNA (200 ng/μL), 1 μL; GmFT5a-616-F (10 μmol/μL), 1 μL; GmFT5a-616-R (10 μmol/μL), 1 μL; ddH₂O 9.5 μL, total volume 25 μL. The PCR reaction was set as follows: 94° C. for 3 min; 94° C. for 30 s, 54° C. for 30 s, and 72° C. for 1 min, for 35 cycles; and a final extension at 72° C. for 10 min. All PCR products were sequenced using GmFT5a-616-R: 5′-TGGGAGACTACAGAAGCAAAGA-3′ (SEQ ID NO: 74) and GmFT5b-661-R: 5′-CAAGACAGGGTTGCTAGGGC-3′ (SEQ ID NO: 72) for GmFT5a and GmFT5b, respectively. Finally, one homozygous Gmft5a Gmft5b double mutant plant was obtained carrying a frameshift mutation in F3 generation. Specifically, there is a 1-bp insertion at target site GmFT5a-TS that resulted in a frameshift-induced premature stop codon in GmFT5a (SEQ ID NO: 53). Meanwhile, there is an 8-bp deletion at target site GmFT5b-TS that resulted in a frameshift-induced premature stop codon in GmFT5b (SEQ ID NO: 55). The progeny of the homozygous ‘Gmft5a Gmft5b’ double mutants comprising both SEQ ID NO: 53 and SEQ ID NO: 55 were all “transgene-free” homozygous Gmft5a Gmft5b double mutants.

### 3.3 Soybean Materials and Growth Conditions

The wild-type (WT) and Gmft5a Gmft5b double mutants were grown and evaluated under short-day (SD; 12 h light and 12 h dark, 22~30° C.) and long-day (LD; 16 h light and 8 h dark, 22~30° C.) in March 2021. The red-to-blue quantum (R:B) ratio of the light was 5.17. The same light as described in Sec. 1.4 of this example above was used.

### 3.4 the Phenotypes of the Gmft5a Gmft5b Double Mutant Plants Differ Under Different Photoperiod Conditions

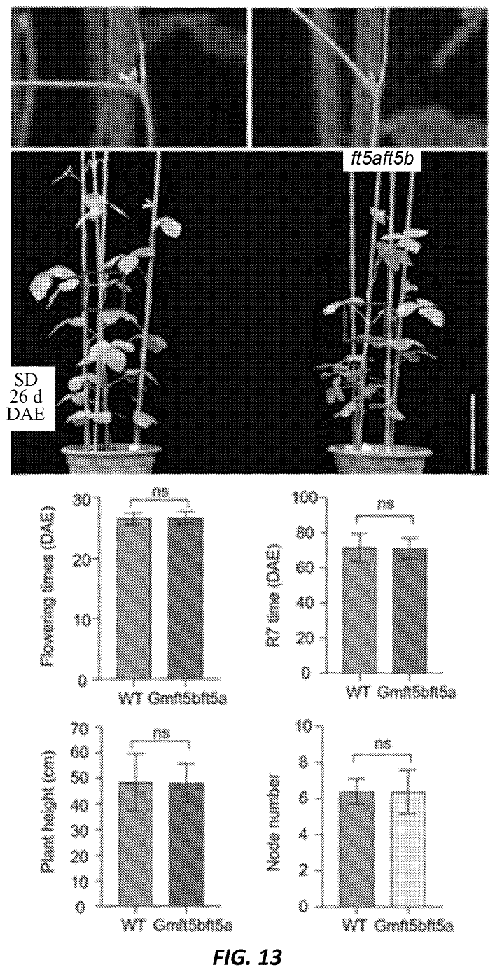

Under SD conditions, the average flowering time of the Gmft5a Gmft5b double mutant plants (26.8±1.0 DAE) was not significantly different from that of WT (26.6±0.96 DAE) (FIG. 13). Besides, the average R7 time of Gmft5a Gmft5b double mutant plants was 71.2±5.8 DAE, which was not significantly different from that of WT (71.7±8.0 DAE). In addition, the average plant height of Gmft5a Gmft5b double mutant plants (48.2±7.6 cm) was not significantly different from WT plants (48.5±11.3 cm). The average node number of Gmft5a Gmft5b double mutant plants (6.4±1.2) was not significantly different from WT plants (6.4±0.7) (FIG. 13, Table 17).

TABLE 17
The phenotypes of WT and Gmft5a Gmft5b double mutant plants
under SD conditions.

| Plants | R1(Flowering)(d) | R7(Maturity)(d) | Plant height(cm) | Node number |
|---|---|---|---|---|
| Gmft5a | 26.8 ± 1.0 | 71.2 ± 5.8 | 48.2 ± 7.6 | 6.4 ± 1.2 |
| Gmft5b | | | | |
| WT | 26.6 ± 0.96 | 71.7 ± 8.0 | 48.5 ± 11.3 | 6.4 ± 0.7 |

Under LD conditions, the average flowering time of Gmft5a Gmft5b double mutant plants (77.1±4.4 DAE) was significantly later than that of WT plants (44.8±1.6 DAE) (FIG. 14). Besides, the average R7 time of WT plants was 146.5±3.9 DAE, while the Gmft5a Gmft5b double mutant plants were not mature for more than 180 days. In addition, the average plant height of Gmft5a Gmft5b double mutant plants (270.0±32.9 cm) was significantly higher than WT plants (221.5±19.8 cm). The average node number of Gmft5a Gmft5b double mutant plants (24.4±1.1) was not different from that of WT plants (22.6±2.5) (FIG. 14, Table 18). One way ANOVA was used for statistics.

TABLE 18
The phenotypes of WT and Gmft5a Gmft5b double mutant plants under
LD conditions.

| Plants | R1(Flowering)(d) | R7(Maturity)(d) | Plant height(cm) | Node number |
|---|---|---|---|---|
| Gmft5a | 77.14 ± .4** | Not | 270.0 ± 32.9* | 24.4 ± 1.1 |
| Gmft5b | | maturity | | |
| | | for more | | |
| | | than | | |
| | | 180 days | | |
| WT | 44.8 ± 1.6 | 146.5+3.9 | 221.5 ± 19.8 | 22.6 ± 2.5 |

**represent p < 0.01.

All patents, patent publications, patent applications, journal articles, books, technical references, and the like discussed in the instant disclosure are incorporated herein by reference in their entirety for all purposes.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams, or the operations described herein without departing from the spirit of the disclosure. For instance, in certain cases, method steps or operations may be performed or executed in differing order, or operations may be added, deleted, or modified.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.” Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

What is claimed is:

1. A plant having a genomic modification, wherein the genomic modification comprises a knock out of one or more of the following genes:
(i) GmCOL2a;
(ii) GmCOL2b;
(iii) GmFT5a;
(iv) GmFT5b; or
(v) GmFT4,
wherein the plant has an altered flowering time and/or maturity time relative to a control plant not comprising the genomic modification.

2. The plant of claim 1, wherein the genomic modification results in decreased expression and/or activity of a polypeptide encoded by the one or more genes, and the decreased expression and/or activity of the polypeptide results in the altered flowering time and/or maturity time under long day (LD) and/or short day (SD) conditions.

3. The plant of claim 1, wherein the genomic modification is non-natural to the plant.

4. The plant of claim 3, wherein said genomic modification comprises a deletion, an insertion, or a substitution in a genomic DNA sequence of said one or more genes.

5. The plant of claim 3 or 4, wherein the genomic DNA sequence for the one or more genes comprises:
(a) at least 85%, 90%, 91%, 92%, 93%, 94%, 95%, 96%, 97%, 98%, or 99% identity to at least one of SEQ ID NO: 15, 21, 27, 52 or 54,
(b) at least 85%, 90%, 91%, 92%, 93%, 94%, 95%, 96%, 97%, 98%, 99%, identity to at least one of the SEQ ID NO: 16, 22, 56, 35, or 39, and/or
(c) a nucleic acid sequences set forth in SEQ ID NO: 1, 6, 43, 49, and 46.

6. The plant of any one of claims 1-5, wherein the genomic modification is accomplished through CRISPR, TALEN, or meganucleases.

7. The plant of claim 6, wherein the genomic modification is accomplished through Cas12a-mediated gene editing.

8. The plant of claim 7, wherein the Cas12a-mediated gene edit employs a gRNA having a target sequence comprising one or more of SEQ ID NOS: 1, 6, 43, 49, or 46.

9. The plant of claim 1, wherein the genomic modification of the one or more genes results in the plant expressing one or more of
(i) a mutant GmCOL2a polypeptide;
(ii) a mutant GmCOL2b polypeptide;
(iii) a mutant GmFT5a polypeptide;
(iv) a mutant GmFT5b polypeptide; and/or
(v) a mutant GmFT4 polypeptide.

10. The plant of claim 1, wherein the genomic modification of the one or more genes results in the plant expressing one or more of
(i) a mutant GmCOL2a allele;
(ii) a mutant GmCOL2b allele;
(iii) a mutant GmFT5a allele;
(iv) a mutant GmFT5b allele; and/or
(v) a mutant GmFT4 allele.

11. The plant of any one of claims 1-10, wherein the genomic modification results in a decrease in expression and/or activity of a polypeptide comprising:
(a) an amino acid sequence comprising at least 85%, 90%, 91%, 92%, 93%, 94, 95%, 96%, 97%, 98%, or 99% identity to at least one of SEQ ID NOS: 17, 23, 28, 36, or 40, and/or
(b) an amino acid sequence set forth in at least one of SEQ ID NOS: 17, 23, 28, 36, or 40.

12. The plant of claim 1 or 9, wherein the genomic modification results in at least 80% decrease in expression of one or more of wild type GmCOL2a polypeptide (SEQ ID NO: 17), wild type GmCOL2b polypeptide (SEQ ID NO: 23), wild type GmFT5a polypeptide (SEQ ID NO: 40), wild type GmFT5b polypeptide (SEQ ID NO: 36), or wild type GmFT4 polypeptide (SEQ ID NO: 28) relative to the control plant not comprising the genomic modification.

13. The plant of claim 9 or 12, wherein the mutant GmCOL2a polypeptide, the mutant GmCOL2b polypeptide, the mutant GmFT5a polypeptide, the mutant GmFT5b polypeptide, or the mutant GmFT4 polypeptide share less than 20% identity with corresponding wild type polypeptides.

14. The plant of claim 9 or 12, wherein the mutant GmCOL2a polypeptide, the mutant GmCOL2b polypeptide, the mutant GmFT5a polypeptide, the mutant GmFT5b polypeptide, or the mutant GmFT4 polypeptide are non-functional polypeptides.

15. The plant of any one of claims 9-14, wherein the plant expresses a mutant GmCOL2a polypeptide comprising: (a) an amino acid sequence that is at least 85% identical to SEQ ID NO: 20, or (b) an amino acid sequence as set forth in SEQ ID NO: 20.

16. The plant of claim 15, wherein the mutant GmCOL2a polypeptide is encoded by a sequence that is at least 85% identical to SEQ ID NO:18 or 19.

17. The plant of any one of claims 9-14, wherein the mutant GmCOL2b polypeptide comprises an amino acid sequence that has at least 85% identity to SEQ ID NO: 26, or wherein the mutant GmCOL2b polypeptide is encoded by a nucleic acid sequence that has at least 85% identity to SEQ ID NO: 24 or 25.

18. The plant of any one of claims 9-14, wherein the mutant GmFT5a polypeptide comprises an amino acid sequence that has at least 85% identity to SEQ ID NO: 42, or wherein the mutant GmFT5a polypeptide is encoded by a nucleic acid sequence that has at least 85% identity to SEQ ID NO: 41 or SEQ ID NO: 53.

19. The plant of any one of claims 9-14, wherein the mutant GmFT5b polypeptide comprises an amino acid sequence that has at least 85% identity to SEQ ID NO: 38, or wherein the mutant GmFT5b polypeptide is encoded by a nucleic acid sequence that has at least 85% identity to SEQ ID NO: 37 or SEQ ID NO: 55.

20. The plant of any one of claims 9-14, wherein the mutant GmFT4 polypeptide comprises an amino acid sequence that has at least 85% identity to SEQ ID NO: 32 or 30, or wherein the mutant GmFT4 polypeptide is encoded by a nucleic acid sequence that has at least 85% identity to SEQ ID NO: 31 or 29.

21. The plant of any one of claims 1-20, wherein the plant is knocked out for one or more of the GmCOL2a gene, the GmCOL2b gene, or the GmFT4 gene, and wherein the plant flowers and/or matures earlier relative to a control plant not comprising the genomic modification under LD conditions.

22. The plant of claim 21, wherein the genetically modified plant flowers and/or matures at least 2 days earlier than the control plant under LD conditions.

23. The plant of claim 21, wherein the genetically modified plant flowers and/or matures 2-40 days earlier than the control plant under LD conditions.

24. The plant of any one of claims 1-22, wherein the plant is knocked out for both the GmCOL2a gene and the GmCOL2b gene, and wherein the plant flowers and/or matures 2-40 days earlier than a control plant under LD conditions.

25. The plant of any one of claims 1-22, wherein the plant is knocked out for the GmFT4 gene, and wherein plant flowers and/or matures 2-40 days earlier than a control plant under LD conditions, and matures 2-40 days earlier than a control plant under SD conditions.

26. The plant of any one of claims 1-20, wherein the plant is knocked out for GmFT5a or GmFT5b, or both GnFT5a and GmFT5b, and wherein the plant flowers and/or matures later relative to a control plant not comprising the genomic modification under LD conditions.

27. The plant of claim 26, wherein the genetically modified plant flowers and/or matures at least 2 days later than the control plant under LD conditions.

28. The plant of claim 27, wherein the genetically modified plant flowers and/or matures 2-40 days later than a control plant under LD conditions.

29. The plant of any one of claims 1-22, wherein the plant is knocked out for both GmFT5a and GmFT5b, and wherein the genetically modified plant flowers and/or matures 30-70 days later than a control plant under LD condition.

30. The plant of any one of claims 1-29, wherein the plant is a dicot plant.

31. The plant of claim 30, wherein the dicot plant is a soybean plant, and optionally wherein the soybean plant is an alite soybean plant.

32. A plant cell, seed, or plant part derived from the plant of any one of claims 9-31, wherein the plant cell, seed, or plant part expresses one or more of the mutant GmCOL2a polypeptide, the mutant GmCOL2b polypeptide, the mutant GmFT5a polypeptide, the mutant GmFT5b polypeptide, or the mutant GmFT4 polypeptide.

33. A harvested product derived from the plant of any one of claims 9-31, or the plant cell, seed, or plant part of claim 32, wherein the harvested product expresses one or more of the mutant GmCOL2a polypeptide, the mutant GmCOL2b polypeptide, the mutant GmFT5a polypeptide, the mutant GmFT5b polypeptide, or the mutant GmFT4 polypeptide.

34. A processed product derived from the harvest product of claim 33, wherein the altered flowering of the genetically modified plant includes fewer days between a VE stage and an R1 stage of the modified plant relative to the control plant.

35. A plant expressing both a mutant GmCOL2a polypeptide and a mutant GmCOL2b polypeptide,
wherein mutant GmCOL2a polypeptide comprises: (a) an amino acid sequence that is at least 85% identical to SEQ ID NO: 20, or (b) an amino acid sequence as set forth in SEQ ID NO: 20, and
wherein the mutant GmCOL2b polypeptide comprises an amino acid sequence that has at least 85% identity to SEQ ID NO: 26, or (b) an amino acid sequence as set forth in SEQ ID NO: 26.

36. A plant expressing both a mutant GmFT5a polypeptide and a mutant GmFT5b polypeptide,
wherein mutant GmFT5a polypeptide comprises: (a) an amino acid sequence that is at least 85% identical to SEQ ID NO: 42, or (b) an amino acid sequence as set forth in SEQ ID NO: 42, and
wherein the mutant GmFT5b polypeptide comprises an amino acid sequence that has at least 85% identity to SEQ ID NO: 38, or (b) an amino acid sequence as set forth in SEQ ID NO: 38.

37. A method of altering flowering time and/or maturity time in a soybean plant, the method comprising, editing in the genome of a soybean plant one or more of the following genes:
(i) GmCOL2a
(ii) GmCOL2b,
(iii) GmFT5a,
(iv) GmFT5b, or
(v) GmFT4,
thereby forming a modified soybean plant, wherein the modified soybean plant has a flowering time and/or maturity time that is altered relative to a control plant not comprising the editing in one or more of the genes.

38. The method of claim 37, wherein the editing includes knocking out of the one or more genes resulting in the modified soybean plant expressing one or more of:
(i) a mutant GmCOL2a polypeptide,
(ii) a mutant GmCOL2b polypeptide,
(iii) a mutant GmFT5a polypeptide,
(iv) a mutant GmFT5b polypeptide, or
(v) a mutant GmFT4 polypeptide.

39. The method of claim 38, wherein the mutant GmCOL2a polypeptide, the mutant GmCOL2b polypeptide, the mutant GmFT5a polypeptide, the mutant GmFT5b polypeptide, or the mutant GmFT4 polypeptide share less than 20% identity with a corresponding wild-type polypeptide.

40. The method of claim 38 or 39, wherein the mutant GmCOL2a polypeptide, the mutant GmCOL2b polypeptide, the mutant GmFT5a polypeptide, the mutant GmFT5b polypeptide, or the mutant GmFT4 polypeptide each is a non-functional polypeptide.

41. The method of claim 38 or 39, wherein the mutant GmCOL2a polypeptide comprises SEQ ID NO: 20, the mutant GmCOL2b polypeptide comprises SEQ ID NO: 26, the mutant GmFT5a polypeptide comprises SEQ ID NO: 42, the mutant GmFT5b polypeptide comprises SEQ ID NO: 38, or the mutant GmFT4 polypeptide comprises SEQ ID NO: 30 or 32.

42. The method of any one of claims 38-40, wherein knocking out of the GmCOL2a gene, the GmCOL2b gene, the GmFT5a gene, the GmFT5b gene, or the GmFT4 gene is performed through gene editing using site-directed nucleases.

43. The method of claim 42, wherein the site-directed nuclease is selected from the group consisting of Cas 12 nuclease, a meganuclease, a zinc-finger nuclease, or a transcription-activator like effector nuclease.

44. The method of any one of claims 38-43, wherein the knocking out of the GmCOL2a gene, the GmCOL2b gene, the GmFT5a gene, the GnFT5b gene, or the GmFT4 gene is performed using Cas nuclease and a guide RNA comprising a nucleotide sequence corresponding to a target sequence in one or more of GmCOL2a gene, the GmCOL2b gene, the GmFT5a gene, the GmFT5b gene, or the GmFT4 gene, respectively.

45. The method of claim 44, wherein the target sequence in the GmCOL2a gene comprises SEQ ID NO: 1.

46. The method of claim 44 or 45, wherein the guide RNA for gene editing of the GmCOL2a gene is encoded by SEQ ID NO: 2 or 3.

47. The method of claim 44, wherein the target sequence in the GmCOL2b gene comprises SEQ ID NO: 6.

48. The method of claim 44 or 47, wherein the guide RNA for genetically modifying the GmCOL2b gene is encoded by SEQ ID NO: 7 or 8.

49. The method of claim 44, wherein the target sequence in the GmnFT5a gene comprises SEQ ID NO: 49.

50. The method of claim 44 or 49, wherein the guide RNA for gene editing the GmFT5a gene is encoded by SEQ ID NO: 50 or 51.

51. The method of claim 44, wherein the target sequence in the GmFT5b gene comprises SEQ ID NO: 46.

52. The method of claim 44 or 51, wherein the guide RNA for gene editing the GmFT5b gene is encoded by SEQ ID NO: 47 or 48.

53. The method of claim 44, wherein the target sequence in the GmFT4 gene comprises SEQ ID NO: 43.

54. The method of claim 44 or 53, wherein the guide RNA for gene editing of the GmFT4 gene is encoded by SEQ ID NO: 44 or 45.

55. The method of any one of claims 37-54, wherein the editing includes knocking out one or more of GmCOL2a, GmCOL2b, or GmFT4, wherein the method further comprises detecting accelerated flowering and/or maturity in the modified soybean plant as compared to a control plant under LD conditions.

56. The method of claim 55, wherein the LD condition is 16 h light/8 h dark in a 24 hour period.

57. The method of claim 55, wherein the accelerated flowering and/or maturity is at least 2 days earlier, at least 4 days earlier, at least 5 days earlier, at least 6 days earlier, or at least 7 days earlier as compared to a control plant grown under LD conditions.

58. The method of any one of claims 37-54, wherein the editing includes knocking out one or both of GmFT5a and GmFT5b, wherein the method further comprises detecting delayed flowering and/or maturity in the modified soybean plant as compared to a control plant under LD conditions, wherein the detecting delayed flowering is based on counting the number of days elapsed between the VE stage and R1 stage, and wherein detecting delayed maturity is based on counting the number of days between the VE stage and the R7 stage.

59. The method of claim 60, wherein the delayed flowering is at least 2 days later, at least 4 days later, at least 5 days later, at least 6 days later, or at least 7 days later as compared to a control plant grown under LD conditions.

60. A modified soybean plant produced using the method of any one of claims 37-59.

61. A plant cell, seed, or plant part derived from the modified soybean plant of claim 60.

62. A method of breeding, comprising:
crossing the plant of any one of claims 10-31 with a different plant not comprising the one or more mutant alleles; wherein both plants are soybean plants, and selecting a progeny plant having altered flowering and/or maturity time.

63. The method of claim 62, wherein the different plant is an elite soybean plant.

64. A plant comprising a genomic modification that results in decreased expression and/or activity of a polypeptide comprising:
(a) an amino acid sequence comprising at least 85%, 90%, 91%, 92%, 93%, 94, 95%, 96%, 97%, 98%, or 99% identity to at least one of SEQ ID NOS: 7, 23, 28, 36, or 40, or
(b) an amino acid sequence as set forth in at least one of SEQ ID NOS: 17, 23, 28, 36, or 40,
wherein the modification is heterologous to the plant and the decreased expression and/or activity in the plant results in the plant having an altered flowering and/or maturity time compared to a control plant not comprising the genomic modification, and wherein the genomic modification is introduced via genome editing.

65. A modified soybean plant, or plant part thereof, comprising one or more non-naturally occurring mutant alleles at one or more loci, wherein the non-naturally occurring mutant allele is introduced via genomic modification using a site directed nuclease, wherein the one or more loci comprise GmFT4a, GmFT5a, GmFT5b, GmCOL2a, or GmCOL2b, and wherein the one or more mutant alleles result in an altered flowering and/or maturity time of the plant relative to a control plant not comprising the mutant allele.

66. The modified soybean plant, or plant part thereof, of claim 65, wherein said non-naturally occurring mutant allele is a homozygous mutant allele.

67. The modified soybean plant, or plant part thereof, of claim 65, comprising a non-naturally occurring mutant allele at each of the GmFT5a locus and the GmFT5b locus, wherein both of said loci comprise homozygous mutant alleles.

68. The modified soybean plant, or plant part thereof, of claim 65, comprising a non-naturally occurring mutant allele at each of the GmCOL2a locus and the GmCOL2b locus, wherein both of said loci comprise homozygous mutant alleles.

69. The modified soybean plant, or plant part thereof, of claim 65, comprising a non-naturally occurring homozygous mutant allele at the GmFT4a locus.

70. The modified soybean plant, or plant part thereof, of any one of claims 65-69, wherein said mutant allele exhibits a reduction of expression or activity relative to an unmodified, wild-type gene allele and wherein the mutant allele results in the modified soybean plant having the altered flowering and/or maturity time when grown under LD conditions.

71. The modified soybean plant, or plant part thereof, of any one of claims 65-70, wherein said mutant allele results in the modified soybean plant having an accelerated flowering and/or maturity relative to the control plant when grown under LD conditions.

72. The modified soybean plant, or plant part thereof, of any one of claims 65-71, wherein at least one of the mutant alleles comprises a nonsense mutation, an in-frame deletion mutation, a missense mutation, a frameshift mutation, a splice-site mutation, or any combination thereof.

73. The modified soybean plant, or plant part thereof, of any one of claims 65-72, wherein at least one of the mutant alleles encodes a protein truncation, a non-functional protein, a protein with reduced function relative to a protein expressed by the corresponding wild type allele, and/or
wherein at least one of the mutant alleles comprises a premature stop codon, a frame-shift mutation, and an in-frame deletion relative to the corresponding wild type allele.
